SEQUENCE LISTING (52 sequences)

SEQ ID NO: 1 - DNA, 1644 bp, Oryza sativa
  source 1..1644: mol_type = genomic DNA
gaaaaaattcccccaaaaccctagcggcggcggcgcgcgctcccctccccacctccgccgccgcggcgtcgaggcagcaccggagggcgatggcgtcgacggtgctggaggcgacgcgggcgaagcacgaggacatggagaggctggagcggctggcggtgcgggagctgcagcgggagcccgccaacgcgcgcgaccgcctctaccagtcccaccgcgtgcgccacatgctcgacctcgtcatctccacctccggcaagctcgtggaaatctacgaggacaaggataatgctaggaaggatgagatctccaaccatcttagttccacagtgcaagctgaaatctttccaaagttctatgacaggctcaaagagatccgtgattaccataggcgtaatccttctgcccgtttcgttagtgcaactgatgatttcgaagagctactaaaggaggaaccggctattgaattcactggagaggaagcttttggccgatacctggacttgcatgagctttacaatgagttcataaattccaagtttggaactccaatggaatactcagcatatgttggcaccttttctcatgtggagaagatggcacaaaatctgaaaacctccaggcaatacagagaatacctggagcatattttggagtatctgacatcatttctgtatcacacagaaccattgcaagacattgagaagatttttgcaaagttggaaagcgagtttgaagaacagtggatcaatggtgaagtgcctggatgggagagcaaggacccagagaaagaatctgcacaagagtcagtaatagaccttgattactataccactgttgaagagcttgttgagctgggcccagaaaagttgaaagaggctttagctgctcgtggattaaaaagtggtggtactgttcaacagcgtgctgagcgacttttcttgttgaagcatacacccttggaacaattggatcgtaagcattttgccaaaggttcacacagttctgtctccaatgctacttctaatggtaacaatttcaaggataatttgaagaaggaaattgctttaatggaagtaaagatgaggcgcctttgtgagctactagatgagatcattgtacggacaaaggaaaatgcagagaagaagctgactctcacatatgaagaaatggaagcagaacgagaagaggaagaagtgcaagctgatagtgaaagtgatgatgaagaccaacaaatctacaatcctcttaagttgccgatgggttgggacggcaaacctataccctattggctatataagctccatggtcttggtcaggaattcaaatgtgagatatgtggaaaccacagctactgggggcgacgggcttatgagcggcatttcaaggagtggcgccatcagcacgggatgcgctgtcttggaattcctaatactaagaacttcaatgaaatcacatccatccaggaggcaaaagaactctgggagaaaattcaacaacgacaagggctgaacaagtggcggccagacctagaagaagagtacgaagatcaggagggaaatatctacaacaagaagacctacactgatctgcagcgccagggcctgatttagaatccctgatgcgatcctaacattttggccc

SEQ ID NO: 2 - DNA, 1524 bp, Oryza sativa
  source 1..1524: mol_type = genomic DNA
atggcgtcgacggtgctggaggcgacgcgggcgaagcacgaggacatggagaggctggagcggctggcggtgcgggagctgcagcgggagcccgccaacgcgcgcgaccgcctctaccagtcccaccgcgtgcgccacatgctcgacctcgtcatctccacctccggcaagctcgtggaaatctacgaggacaaggataatgctaggaaggatgagatctccaaccatcttagttccacagtgcaagctgaaatctttccaaagttctatgacaggctcaaagagatccgtgattaccataggcgtaatccttctgcccgtttcgttagtgcaactgatgatttcgaagagctactaaaggaggaaccggctattgaattcactggagaggaagcttttggccgatacctggacttgcatgagctttacaatgagttcataaattccaagtttggaactccaatggaatactcagcatatgttggcaccttttctcatgtggagaagatggcacaaaatctgaaaacctccaggcaatacagagaatacctggagcatattttggagtatctgacatcatttctgtatcacacagaaccattgcaagacattgagaagatttttgcaaagttggaaagcgagtttgaagaacagtggatcaatggtgaagtgcctggatgggagagcaaggacccagagaaagaatctgcacaagagtcagtaatagaccttgattactataccactgttgaagagcttgttgagctgggcccagaaaagttgaaagaggctttagctgctcgtggattaaaaagtggtggtactgttcaacagcgtgctgagcgacttttcttgttgaagcatacacccttggaacaattggatcgtaagcattttgccaaaggttcacacagttctgtctccaatgctacttctaatggtaacaatttcaaggataatttgaagaaggaaattgctttaatggaagtaaagatgaggcgcctttgtgagctactagatgagatcattgtacggacaaaggaaaatgcagagaagaagctgactctcacatatgaagaaatggaagcagaacgagaagaggaagaagtgcaagctgatagtgaaagtgatgatgaagaccaacaaatctacaatcctcttaagttgccgatgggttgggacggcaaacctataccctattggctatataagctccatggtcttggtcaggaattcaaatgtgagatatgtggaaaccacagctactgggggcgacgggcttatgagcggcatttcaaggagtggcgccatcagcacgggatgcgctgtcttggaattcctaatactaagaacttcaatgaaatcacatccatccaggaggcaaaagaactctgggagaaaattcaacaacgacaagggctgaacaagtggcggccagacctagaagaagagtacgaagatcaggagggaaatatctacaacaagaagacctacactgatctgcagcgccagggcctgatttag

SEQ ID NO: 3 - protein, 507 aa, Oryza sativa
MASTVLEATRAKHEDMERLERLAVRELQREPANARDRLYQSHRVRHMLDLVISTSGKLVEIYEDKDNARKDEISNHLSSTVQAEIFPKFYDRLKEIRDYHRRNPSARFVSATDDFEELLKEEPAIEFTGEEAFGRYLDLHELYNEFINSKFGTPMEYSAYVGTFSHVEKMAQNLKTSRQYREYLEHILEYLTSFLYHTEPLQDIEKIFAKLESEFEEQWINGEVPGWESKDPEKESAQESVIDLDYYTTVEELVELGPEKLKEALAARGLKSGGTVQQRAERLFLLKHTPLEQLDRKHFAKGSHSSVSNATSNGNNFKDNLKKEIALMEVKMRRLCELLDEIIVRTKENAEKKLTLTYEEMEAEREEEEVQADSESDDEDQQIYNPLKLPMGWDGKPIPYWLYKLHGLGQEFKCEICGNHSYWGRRAYERHFKEWRHQHGMRCLGIPNTKNFNEITSIQEAKELWEKIQQRQGLNKWRPDLEEEYEDQEGNIYNKKTYTDLQRQGLI

SEQ ID NO: 4 - DNA, 2071 bp, Oryza sativa
  source 1..2071: mol_type = genomic DNA
ttgaacggaagagaagaaagatggcgggcaacaagggagagggccctgcgatcggcatcgacctcggcacgacctactcgtgcgtcggcgtgtggcagcatgacagggtggagatcatcgccaacgaccagggcaaccgcaccacgccgtcgtacgtggccttcaccgacaccgagaggctcatcggcgacgccgccaagaaccaggtcgccatgaaccccaccaacaccgtctttgatgcaaagcggctgatcgggcggcgcttctcggacccgtcggtgcaggcggacatgaagatgtggccgttcaaggtggtgcctggcccagccgacaagccgatgatcgtggtgacgtacaagggcgaggagaagaagttctcggcggaggagatctcgtccatggtgctcaccaagatgaaggagatcgccgaggccttcctcagcaccaccatcaagaacgccgtcatcaccgtcccggcctacttcaacgactcccaacgccaggccaccaaggacgccggcgtcatctccggactcaacgtcatgcgcatcatcaacgagcccaccgccgccgccatcgcctacggcctcgacaagaaggccgccagcaccggcgagaagaatgtcctcatcttcgacctcggcggcggcaccttcgacgtctccatcctcaccatcgaggagggcatcttcgaggtcaaggccaccgccggcgacacccacctgggaggcgaggacttcgacaaccggatggtgaaccacttcgtgcaggagttcaagaggaagcacaagaaggacatcaccggcaacccgagggcgctccggcggctgaggacggcgtgcgagagggcgaagaggacgctgtcctccaccgcccagaccaccatcgagatcgactcgctctacgagggcatcgacttctacgccaccatcacccgcgcccgcttcgaggagctcaacatggacctcttccgccggtgcatggagcccgtggagaagtgcctccgcgacgccaagatggacaaggcccagatccacgatgtcgtcctcgtcggaggctccacccgtatccccaaggtgcagcagctcctgcaggacttcttcaacgggaaggagctctgcaagagcatcaaccccgacgaggccgtggcgtacggcgccgccgtccaggccgccatcctcagcggcgagggcaaccagagggtgcaggacctgctcctcctcgacgtcacgccgctctcgctcgggttggagacggccgggggtgtcatgaccgtgctgatcccgaggaacaccaccatcccaaccaagaaggagcaggtcttctccacctactccgacaaccagcccggcgtgctcatccaggtgtacgagggcgagaggacgaggaccaaggacaacaacctgctcggcaagttcgagctcaccggcatcccgccggcgccgaggggcgtgccccagatcaacgtgacgttcgacatcgacgcgaacggcatcctgaacgtgtcggcggaggacaagacgacggggaagaagaacaagatcaccatcaccaacgacaaggggcggctgagcaaggaggagatcgagcggatggtgcaggaggccgagaagtacaaggccgaggacgagcaggtgcggcacaaggtggaggcccgcaacgcgctggagaactacgcgtacaacatgcgcaacacggtgcgcgacgagaagatcgcctccaagctccccgccgacgacaagaagaagatcgaggacgccatcgaggatgccatcaagtggctcgacggcaaccagctcgccgaggccgacgagttcgaggacaagatgaaggagctcgagagcctctgcaatccgatcatctcaaagatgtaccagggcggcgccggtggtccggcgggcatggacgaagacgcccccaacggcagtgccggcaccggcggtggcagcggcgctggccccaagatcgaggaagtggactaagcgagtggactgagtatatgacatgttgagcgggacgaagaaggagcgatgcattctgggacgtcatcatcatctttagagtttcatgc

SEQ ID NO: 5 - DNA, 1962 bp, Oryza sativa
  source 1..1962: mol_type = genomic DNA
atggcgggcaacaagggagagggccctgcgatcggcatcgacctcggcacgacctactcgtgcgtcggcgtgtggcagcatgacagggtggagatcatcgccaacgaccagggcaaccgcaccacgccgtcgtacgtggccttcaccgacaccgagaggctcatcggcgacgccgccaagaaccaggtcgccatgaaccccaccaacaccgtctttgatgcaaagcggctgatcgggcggcgcttctcggacccgtcggtgcaggcggacatgaagatgtggccgttcaaggtggtgcctggcccagccgacaagccgatgatcgtggtgacgtacaagggcgaggagaagaagttctcggcggaggagatctcgtccatggtgctcaccaagatgaaggagatcgccgaggccttcctcagcaccaccatcaagaacgccgtcatcaccgtcccggcctacttcaacgactcccaacgccaggccaccaaggacgccggcgtcatctccggactcaacgtcatgcgcatcatcaacgagcccaccgccgccgccatcgcctacggcctcgacaagaaggccgccagcaccggcgagaagaatgtcctcatcttcgacctcggcggcggcaccttcgacgtctccatcctcaccatcgaggagggcatcttcgaggtcaaggccaccgccggcgacacccacctgggaggcgaggacttcgacaaccggatggtgaaccacttcgtgcaggagttcaagaggaagcacaagaaggacatcaccggcaacccgagggcgctccggcggctgaggacggcgtgcgagagggcgaagaggacgctgtcctccaccgcccagaccaccatcgagatcgactcgctctacgagggcatcgacttctacgccaccatcacccgcgcccgcttcgaggagctcaacatggacctcttccgccggtgcatggagcccgtggagaagtgcctccgcgacgccaagatggacaaggcccagatccacgatgtcgtcctcgtcggaggctccacccgtatccccaaggtgcagcagctcctgcaggacttcttcaacgggaaggagctctgcaagagcatcaaccccgacgaggccgtggcgtacggcgccgccgtccaggccgccatcctcagcggcgagggcaaccagagggtgcaggacctgctcctcctcgacgtcacgccgctctcgctcgggttggagacggccgggggtgtcatgaccgtgctgatcccgaggaacaccaccatcccaaccaagaaggagcaggtcttctccacctactccgacaaccagcccggcgtgctcatccaggtgtacgagggcgagaggacgaggaccaaggacaacaacctgctcggcaagttcgagctcaccggcatcccgccggcgccgaggggcgtgccccagatcaacgtgacgttcgacatcgacgcgaacggcatcctgaacgtgtcggcggaggacaagacgacggggaagaagaacaagatcaccatcaccaacgacaaggggcggctgagcaaggaggagatcgagcggatggtgcaggaggccgagaagtacaaggccgaggacgagcaggtgcggcacaaggtggaggcccgcaacgcgctggagaactacgcgtacaacatgcgcaacacggtgcgcgacgagaagatcgcctccaagctccccgccgacgacaagaagaagatcgaggacgccatcgaggatgccatcaagtggctcgacggcaaccagctcgccgaggccgacgagttcgaggacaagatgaaggagctcgagagcctctgcaatccgatcatctcaaagatgtaccagggcggcgccggtggtccggcgggcatggacgaagacgcccccaacggcagtgccggcaccggcggtggcagcggcgctggccccaagatcgaggaagtggactaa

SEQ ID NO: 6 - protein, 653 aa, Oryza sativa
MAGNKGEGPAIGIDLGTTYSCVGVWQHDRVEIIANDQGNRTTPSYVAFTDTERLIGDAAKNQVAMNPTNTVFDAKRLIGRRFSDPSVQADMKMWPFKVVPGPADKPMIVVTYKGEEKKFSAEEISSMVLTKMKEIAEAFLSTTIKNAVITVPAYFNDSQRQATKDAGVISGLNVMRIINEPTAAAIAYGLDKKAASTGEKNVLIFDLGGGTFDVSILTIEEGIFEVKATAGDTHLGGEDFDNRMVNHFVQEFKRKHKKDITGNPRALRRLRTACERAKRTLSSTAQTTIEIDSLYEGIDFYATITRARFEELNMDLFRRCMEPVEKCLRDAKMDKAQIHDVVLVGGSTRIPKVQQLLQDFFNGKELCKSINPDEAVAYGAAVQAAILSGEGNQRVQDLLLLDVTPLSLGLETAGGVMTVLIPRNTTIPTKKEQVFSTYSDNQPGVLIQVYEGERTRTKDNNLLGKFELTGIPPAPRGVPQINVTFDIDANGILNVSAEDKTTGKKNKITITNDKGRLSKEEIERMVQEAEKYKAEDEQVRHKVEARNALENYAYNMRNTVRDEKIASKLPADDKKKIEDAIEDAIKWLDGNQLAEADEFEDKMKELESLCNPIISKMYQGGAGGPAGMDEDAPNGSAGTGGGSGAGPKIEEVD

SEQ ID NO: 7 - DNA, 3411 bp, Oryza sativa
  source 1..3411: mol_type = genomic DNA
ccttttcttagtgggcttcataacgcgagccccatgccggaatacgatccggcccatgggccaatgggagtccgtttcgtcttcgtcttcgtcgtcgtcgtcttcttttcttgcaagaaagaaaaggcggagcaggagcagcaacgagaaaaggcggaggcggcggcgcaaccgacaaggaaaagcaaagcgagagcaggaaaacctcgccggcggcgacaccaccaccaccagcaggcagggatggcccggtacgaccgcgcgatcaccgtcttctcccccgacggccacctcttccaggtggagtacgccctcgaggccgtccgcaagggcaacgccgccgtcggcgtccgcggcaccgacaccgtcgtcctcggcgtcgagaagaaatccacccccaagctccaggactccaggtaaatccttcaacccgccccctccccttcgccggatctcacccgcggatcgatctcccccccaaatccaggtccatgcgcaagatcgccagcctcgacacccacatcgcgcttgcctgcgcggggctcaaggcggacgcccgcgtgctcatcaaccgggcgcgcgtcgagtgccagagccaccgcctcaccgtcgaggaccccgtcaccgtcgagtacatcacgcgctacatcgccggactgcagcagaagtacacccagagcggcggggtgcgccccttcggcctctccaccctcatcgtcggcttcgacccctacaccgagaagcccgccctctaccagaccgacccatccggcaccttctccgcctggaaggccaacgccaccggccgcaactccaactccatgcgcgagttcctcgagaagaattacaaggacacctccggcaaggagaccatcaagcttgccatccgagccctccttgaggtatatatatacacttcacactgaataattttactagttaaaactacctctagctttgatccactaccactgaatcagtaactgtgcttctgaagacagatactcaaaagtaattcactaatttgtgttgcttatgttgactggttcagtaatttgtaagatgcccgcctacgattgaacatcgttatcttcgttaagttccatatgactcaatttggtgcaaagattaatagttccctgaatctttgttacaatcggctgagactataaatgcagtcatgaacctaaaatagagtgggatggatgggattggttttattgtgtttgcttccatcttaaagatgatacttattggtgcctggagtgatcatagcaattggtatggcttggatctgtaatctcataaatcatctccagctatttcagcatttgtatattggttttcatctgcacctcaaattttattgttgtagtgttggctataggtctttaactctttatccgtagtaaatcgtctctagctatttaatcatttgtgtattttgattgtcatctgcatctcatattttaagtgttaagcacggtttcaaaagtttttaaaggcattcttatgtgagtacatttttctcttcaaaagttattaaagttgtttgattagtggcatataaacttttgtttttctgttattgataatctgcacctgtagttctcttctattgcactgtagaagctgcaacttgcatgtattttgtcaactctgctgccgctttagtatgttgctgcttagatataggcacggatgcctagttcattgtatttactgtctaggtaaaaaaaatgtatatacaccattacaaaaatacatatcaaattaaggtcatctttgtttaggcttagacttattggcttgagattttaagtcagcttattggcttatatgttttataaaccggtggatttgaaatcctaagttcaatggttgagtcatacctccacttcatataatccaacaaaatggtttctccaacctagcttttggcttaacagtgtaagagtggcttgtggctttaaaaagggcaaacaaataagctgcttgtttgtttaggcttaggctttttggctcataagctagcttataagcctaaacaaagaaggcctaagtctaaatgtccttttttcattactgtaagtctctaactgcaaatggttatgttcctcaaggacacatagttcatagctaaatacttcaaattaagttcttagcctgcatctggttatatattcgtttattggtggcttcaaatggttaacacatatgcacagccaccattgagtttatgctagctaatggtaagcagttgatcatttttgcaatctgttgctcatacagaccgtgaaaatgcacgtctcttgcacattttgtcatatacgtatatgtctgtacctcttttagtgaaatatcttgctttttttcgttgttggaagacaaagtagtatgttagtactttgtttcatgctttgccagttgccacatatgacaaactcattgaccttttttccattggtcagtggcagaggtagatgggtacatgggggtacatgtgtaccccaaactctttgctaaccaaccacactatacataaacatatacaatttatactcaaataagaaaactagctattcaagggaatgaagagtcacttattttattagatttgaactttcatgaatctatataccatatatgaaccaatttgataatttactggtaaatgtcactatattctaataatcatgccacttaggtcagattctgaacccctcattaagaatattctggctccaccaatgccattggttatcacatatgcacagttcatgatatccgaaatcaatttcttactctgcttatacattcattggtggcttcaaatggttatcacatatgcatattaactattgagtccatggtacttatttctttataatgacattactactgccatttgataggtaattaagcattttgtcagtctgttgcacatgtagaattttctgatgagctcttttagtgcaacaaatgtcatccttttttacttgtctgtagacagagatgcctgcttgctttgctttttttttttcatgacgaactcattggcctttgacctttgtttattcagtgttttttgctgagttggtttctccatcatagctatatcatttgatgcttggcattttatatgcaacaaatatcttaaatttgagtaatgggttgatgtctccaggttgtcgagagcggtggcaagaacatcgagattgcggtgatgacacacaaggatggcctccgcgagctggaggaggctgagatcgacgagtacgtggctgagattgaagcagagaaggctgccgccgaggccgcgaagaagggcgccccgaaagaaacctgacctgatcatcatgattcagcaaagcataatctctcttagcatgtgttgtttcttttctgttc

SEQ ID NO: 8 - DNA, 960 bp, Oryza sativa
  source 1..960: mol_type = genomic DNA
atgccggaatacgatccggcccatgggccaatgggagtccgtttcgtcttcgtcttcgtcgtcgtcgtcttcttttcttgcaagaaagaaaaggcggagcaggagcagcaacgagaaaaggcggaggcggcggcgcaaccgacaaggaaaagcaaagcgagagcaggaaaacctcgccggcggcgacaccaccaccaccagcaggcagggatggcccggtacgaccgcgcgatcaccgtcttctcccccgacggccacctcttccaggtggagtacgccctcgaggccgtccgcaagggcaacgccgccgtcggcgtccgcggcaccgacaccgtcgtcctcggcgtcgagaagaaatccacccccaagctccaggactccaggtccatgcgcaagatcgccagcctcgacacccacatcgcgcttgcctgcgcggggctcaaggcggacgcccgcgtgctcatcaaccgggcgcgcgtcgagtgccagagccaccgcctcaccgtcgaggaccccgtcaccgtcgagtacatcacgcgctacatcgccggactgcagcagaagtacacccagagcggcggggtgcgccccttcggcctctccaccctcatcgtcggcttcgacccctacaccgagaagcccgccctctaccagaccgacccatccggcaccttctccgcctggaaggccaacgccaccggccgcaactccaactccatgcgcgagttcctcgagaagaattacaaggacacctccggcaaggagaccatcaagcttgccatccgagccctccttgaggttgtcgagagcggtggcaagaacatcgagattgcggtgatgacacacaaggatggcctccgcgagctggaggaggctgagatcgacgagtacgtggctgagattgaagcagagaaggctgccgccgaggccgcgaagaagggcgccccgaaagaaacctga

SEQ ID NO: 9 - protein, 319 aa, Oryza sativa
MPEYDPAHGPMGVRFVFVFVVVVFFSCKKEKAEQEQQREKAEAAAQPTRKSKARAGKPRRRRHHHHQQAGMARYDRAITVFSPDGHLFQVEYALEAVRKGNAAVGVRGTDTVVLGVEKKSTPKLQDSRSMRKIASLDTHIALACAGLKADARVLINRARVECQSHRLTVEDPVTVEYITRYIAGLQQKYTQSGGVRPFGLSTLIVGFDPYTEKPALYQTDPSGTFSAWKANATGRNSNSMREFLEKNYKDTSGKETIKLAIRALLEVVESGGKNIEIAVMTHKDGLRELEEAEIDEYVAEIEAEKAAAEAAKKGAPKET

SEQ ID NO: 10 - DNA, 26 bp, synthetic construct
  misc_feature 1..26: note = Forward primer for cloning cDNA of OsZOS3-17 gene
  source 1..26: mol_type = other DNA
gaaaaaattcccccaaaaccctagcg

SEQ ID NO: 11 - DNA, 24 bp, synthetic construct
  misc_feature 1..24: note = Reverse primer for cloning cDNA of OsZOS3-17 gene
  source 1..24: mol_type = other DNA
gggccaaaatgttaggatcgcatc

SEQ ID NO: 12 - DNA, 24 bp, synthetic construct
  misc_feature 1..24: note = Forward primer for cloning cDNA of OsDnaK gene
  source 1..24: mol_type = other DNA
ttgaacggaagagaagaaagatgg

SEQ ID NO: 13 - DNA, 27 bp, synthetic construct
  misc_feature 1..27: note = Reverse primer for cloning cDNA of OsDnaK gene
  source 1..27: mol_type = other DNA
gcatgaaactctaaagatgatgatgac

SEQ ID NO: 14 - DNA, 26 bp, synthetic construct
  misc_feature 1..26: note = Forward primer for cloning cDNA of OsPPT1-1 gene
  source 1..26: mol_type = other DNA
ccttttcttagtgggcttcataacgc

SEQ ID NO: 15 - DNA, 28 bp, synthetic construct
  misc_feature 1..28: note = Reverse primer for cloning cDNA of OsPPT1-1 gene
  source 1..28: mol_type = other DNA
gaacagaaaagaaacaacacatgctaag

SEQ ID NO: 16 - DNA, 20 bp, synthetic construct
  misc_feature 1..20: note = Forward primer for real-time RT-PCR analysis of OsZOS3-17 gene
  source 1..20: mol_type = other DNA
aggcaaaagaactctgggag

SEQ ID NO: 17 - DNA, 22 bp, synthetic construct
  misc_feature 1..22: note = Reverse primer for real-time RT-PCR analysis of OsZOS3-17 gene
  source 1..22: mol_type = other DNA
ctgcagatcagtgtaggtcttc

SEQ ID NO: 18 - DNA, 20 bp, synthetic construct
  misc_feature 1..20: note = Forward primer for real-time RT-PCR analysis of OsDnaK gene
  source 1..20: mol_type = other DNA
atcgaggatgccatcaagtg

SEQ ID NO: 19 - DNA, 20 bp, synthetic construct
  misc_feature 1..20: note = Reverse primer for real-time RT-PCR analysis of OsDnaK gene
  source 1..20: mol_type = other DNA
cgccctggtacatctttgag

SEQ ID NO: 20 - DNA, 21 bp, synthetic construct
  misc_feature 1..21: note = Forward primer for real-time RT-PCR analysis of OsPPT1-1 gene
  source 1..21: mol_type = other DNA
gtggcaagaacatcgagattg

SEQ ID NO: 21 - DNA, 21 bp, synthetic construct
  misc_feature 1..21: note = Reverse primer for real-time RT-PCR analysis of OsPPT1-1 gene
  source 1..21: mol_type = other DNA
ttcaatctcagccacgtactc

SEQ ID NO: 22 - DNA, 27 bp, synthetic construct
  misc_feature 1..27: note = Forward primer for PCR analysis of OsZOS3-17 gene
  source 1..27: mol_type = other DNA
atttggagaggacacgctgaaatcacc

SEQ ID NO: 23 - DNA, 22 bp, synthetic construct
  misc_feature 1..22: note = Reverse primer for PCR analysis of OsZOS3-17 gene
  source 1..22: mol_type = other DNA
cggaggtggagatgacgaggtc

SEQ ID NO: 24 - DNA, 27 bp, synthetic construct
  misc_feature 1..27: note = Forward primer for PCR analysis of OsPPT1-1 gene
  source 1..27: mol_type = other DNA
atttggagaggacacgctgaaatcacc

SEQ ID NO: 25 - DNA, 20 bp, synthetic construct
  misc_feature 1..20: note = Reverse primer for PCR analysis of OsPPT1-1 gene
  source 1..20: mol_type = other DNA
ggtggatttcttctcgacgc

SEQ ID NO: 26 - DNA, 25763 bp, Zea mays
  source 1..25763: mol_type = genomic DNA
cccagacgggcccaaatctatcgcaaacaggcctcgtctcgtcctcgtcctcgtccgccgccaaatcgaacccccccaaaccctagctacccggcgcaacgtccccttcccgcctctccgcggcgctgcgtgacacccggcctccgctacgaaacaccggggcgggtgtagcctagcgggcgggccatggcgtcaacggtgctggaggcgacgcgggcggcccatgaggatctggagaggctggagcggctcgcggtgagcgagctgcaacgggagcccgctaacccgcgtgaccgcctctttcagtcccaccgcgtccgccacatgctcgacctcgtcgtctccacctccggcaagctcgtacgtgccattcttctgcttctaaggattcacgactccatccacttcacttcagtgattcgttttgcttagctgggtcaggttcgaacacggtcgataggattgatatttgcagttgtttctgtcgatttcggatgcttgtttagggttggcttctgagtgtatgaggaattacagatccgtcttcctggcaaattcttattacatatcaatcatgttatgtttatctctgaattcgatctgtgtagatggattttgtgctcttatttttactgtagttggcagtggcactgcctgattagtgttgttcaccggaggttttggctagtgttatttctgtaccgttctgtccccttcattctcttttggtctgagtcagtagttgacctttagaaatcttaattttgctgtccagcgacgatctcacttttgctctggtgcttgtaacttgtttaggtggaaatttacgaggacaaggacaatgctaggaaggatgagatcaacactcatcttactgcacccacgcaaagtggactgtttagcaagtactatgaaaggctcaaagaggtaccttacctatctgcttgcttggatgtgacaaaagtgaaatagatgccagtactcattactcattcaccaaaggtcatgatgatgcttatcatgttctttttagtcgagttcttctccgcttttaagaaaacagtaatgtacggcacaaactatatggctggaggtaccagtttgcaaacacaaacaaaagcaaacaagaaagaattgtcctgttacaatgaaactaaagctctgtaatgttcgggacaggaataggtgaaggcctgaagttgtccatccagttccaagtgatcttatccactctaatgtggtcttttggttttagtagttgtagctccatctactgcagaaacataaaacagcacaccagaggtgtggctaggaaatttcctctcaaatctcatcttattgcatgatgcatgctttccacattgacaatgccaagtcttcaaagaaaaaaaaagtatgtacgttggggtaaatttaggttagttatagcctaacttcccatgaagtcagatctagaaatgggatgaaagtcccaaccaaaagcctctgtaaaactgcaccatgcagatctagcaatagggcatctgaacttctgaagaacatatggcctatgttctcaggttcattagagagaacatgccatttctgggccccttctttttgaaactgttgtggcctgtaatttaccatagaaaaattgataattttcatgggcgcttttgatttccaaagttcttttgtcatactgtcactgacacctccatcagttaaaagtctatagagatttggtagtgaactgcagccatcaaatccacccatggattgtagtcagatgaggatagaaacccacttaaatttgtaccccaatcatcttcagttcaatattcaatcactgtagcatctggttcttcacaaatcctatgtaactcagggtactcggtttttttgaatgcaatggtaggagctctgcctctcaattaagaagaatagaattgacccagttttaaggaaaaatcgggcccaaaacctaacagagcacacaacagagtaaccggcggaaggcccccacaaacaacctagacaacggtagccccacactcacaaaccccatctagagtcattgttgacaagctcaacacaagggatgagcaggtcccacttctcaagacgagctgcacacagccaaaggaactcgacgacacctacacccaaataccgacacacaaggaagaaggccgcgacacgccattgttgccaagcctcgacacgccccacgcgaggtagctccgattcttcaccacgagcaatgttccacatcgccgaacactgggagcagatagatgcctagagcgacaaaggcccaggtaccaccgagccaagcggagtgacgaaaagcctccacaacgccgacaaccacacaaaactggaggtcttccgggcaacgtcttcaaggagaaacgacgtcgagacgccgccgtcgcccgcctggaacaatccaagcaaagttttctccgaagatctagcagagcaacgttgattgccagcaccaaggagaaaactgcagggacagccacaccgatgtcaccccaccaagccgcccggcgttgtctccttctgccacccaccgacgcaccaaccctcagagcctcgacgccggccgagcaccagcgaggaaccccaagaaggccgaggcgaggactcccacacggagagcgagagatctaggacgtcaccctcaacagggtaggagtggcacccgtaggcgtcaccgacgtccgcccgtaaatgggcatcgctttcgccggaggacctcccaccacatgtcccaacggacggagccagccagcccttcccacatcaccgaccgagaagagaggtggagagatggatctggccggagaggctcaaaatttagcctccaaccgatcgtggagcatgatctgaagccacgacgccgggaaccgccgaggctgccgtcccatgacccaccagctgcagccctgccatatttacattgccgctggcagctgcagtccgccgctaggccaaggcccacgcgggccacgtcctcgacccaaccaccgtgccacgagcacggacagggcgcaaccgccgctgtgggctagccacagctatgcgagctggcatggccgccgtcgccaggcggcctaggccaccctcctaggtcggcggggagggagaggggtggggagtggcggagcgagggggagcatctcgctgctgccgccgtccttgcagggcgcgcgggaagcgccgcgtccctgctctggcagtggcgaggccaagtcgtggcgggggggggggaggctagggttctaagcacagcgccgcgagcccatatatcttcccataaaaaaatcttcctgctattgcatgccttgcaaataactccacatttgaacaaatatgtaaaacctgtgtaggcccttatgtttgtaaactgctccacgtttgaacagatgtttaaacttatgtacagagaattgcctttatgcttggaagtaaatgagctattgctgcgtttggatgtcgatattggagggcatggatttgaatcgggttgaataccaaatcagccatggtattgaaatgagatataattccaattctattgtttggatgtcactaaattggagtttggaatggtgcggtctaattccatgcaataccgaggggtgaggccttgtattgggatatggagtttctagttatagtccaattccagggaattgggtctctgatcccaaatctcaattccacgtgcaaccaaacaacgaaattttggaaagttgattccaattcgcaattctgtgctccaatatctacatccaaacgaggtatacgagttctcatttggcatgtatttggtttgaagaattcttaaccaaggattatttgagccttgcacatatttccaaatccattcataatcttggtctcattgtcccaagaccaccttggtcctttggtctgactatcattcccaacttagccatatgttatgttgttgaatttatcttatgcacttcgctagaagatttctgacctgatagagttcatcacttcatcttattgtgaactctgctaattgtaaaatcacactgtatctatataggcatatgttaaccttcccaaggttaaacttttacccttccatgggtctagcctcctgagagcacctagaggggggggggtgaataggtgatcctgaataattcaaacttatagccacaaaaacttgttaagcgttagcacagttaatgccaagtggctagagcgaagatcaatatgataatcacaaagagttcaacacagagaagacacagtgatttatcccgtggttcggccaagtacaaaacttgcctactccacgttgtggcgtcccaacggacgagagttgcactcaactcctcaagtgatccaatgatcaacttgaataccacagtgttatgcttttcttttcttatcgcgtttgtgaggaatctccacaacttggagtctctcacccttacacttgaagttcacaaagaagcacggagtaagggagagaagcaacacacacaaatccacagcaaaatgcgcgcacacacggccaagaatcgagctcaaaagactatctcaaagttctcactagaacggagctcgaatcactgagaatgacaaacgaatgcgcaaagactgagtgtggatgatcaagaatgctctaaggttgcttggtctcttcctccatgcgcctaggggtcccttttatagccccaaggcagctaggagccgttgagagtaaaactggaaggctgatcttgccttctgtcgactggtgcaccggacagtccggtgcacaccggacactgtccggtgcccgatttctttccttaaatggcgcagtcgaccgttgcagatctgggagccgttggcgcaccggacatgtccggtgcacaccggacagtccggtgcccccttccgaccgttggctcggccacgtgtcccgcgcagattgcgcggccgaccgttggctcaccggacagtccggtgcacaccggacagtccggtgaattatagccgtacgccgtcggcgaaatcccgagagcggccacttcatgccgagtcagcctggcgcaccggacactgtccggtgcaccaccggacaatccggtgtgccagactgagctgagtcttggctgtacacagccaagcttttgcacctctcttcttttctgtttctaacacttagacaaatatattagtacacaaaaccaatgtactaaggcttagaaacatacctttactcttgatttgcactttgttcatccatgggcataaattcacatttaagcacttgtgtttgcactcaatcaccaaaatacttagaaatggcacaagagctcatttccctttcaatctccccctttttggtgatttatgccaacacaacataaagcaactataacaagtgcaatatcacttcaaataaaaactcagttttattttgattcaattttggcatatatggatcatcctttgccaccacttggtttgtttttgcaaatcaaactcaaatctctatctctaagtcaaacacacatgttgaaacataaagagagtcattccaaaagagattgatcaaggatttcaaaaactccccctttttcccataatcactacttctccccacaagaagccaacttttgacaaaagagacaatgaaagagttttgacaaaccaaaaactctactctactattttcaaatctctcaagtggtagctgatccatttttcactttggcctttattttctccccctttggcatcaagcaccaaaacggaatcaatcttggccctttaaccccattgcctcaccaaaatcttcaattaagagcaaatggcaataagagttcatgagatgaacttggaattagttaccctctcatcggagtgcagtggaagtctttcatggtccaagtccaccgtttctctttcaattctcctttgagactaaatcaagcaaactcaagcatatggttagtctcaaagggtcaagttgtaacacatctccccctaaacatgtgcatcactttgcaacggacttgtgaggtccggggagggtttgtacaacttgagcaccacaataagcaacaaaatgcagaatgaacatgatcaaaggcataaacacatgtatgctacaattcaatccaagttccgcgaatctaggacatttagctcactacgcagcttgcaaaaggtcttctcatctagaggcttggtaaagatatcggctagctggttctcggtgctaacatgaaacacttcgatatctcccttttgctggtggtctctcaaaaagttatgccggatgtctatgtgctttgtgcggctgtgctcaacaggattttccgccatgcggatagcactctcattatcacataggagtgggactttgctcagattgtagccaaagtccctgagggtttgcctcatccaaagtagttgcgcgcaacactgtcctgcggcaacgtactcggcctcagcggtggatagggcaacagaagtttgtttcttagagttccacgacaccagggaccttccctaagaattggcacgtccccgatgtactcttcctatcgaccttacatccagcatagtcggaatctgagtatccaaccaagtcaaaggtagaccccttttggataccagagtccgaagcaaggcgtagcaaccaaatatctaagaattcgcttcaccgccactaagtgacactccttaggatcggattgaaatctagcacacatgcatacgctaagcataatatccggtctactagcacataagtaaagtaatgaccttatcattgaccggtatgccttttgatcaacggacttacctcctttgttgaggtcggtgtgtccgtcggttcccatcggagtctttgcgggtttggcgtccttcatcccaaaccgcttcagcaaatcttgcgtgtatttcgtttgggagatgaaggtgccgtccttgagttgcttcacttggaacccaaggaagtagttcaactcgcccatcattgacatctcgaatttctgcgtcatcaccctgctaaactcttcacaagacttttattagtagaaccaaatattatgtcatcgacataaatttggcatacaaacaaatcaccatcacatgtcttagtgaaaagagttggatcggctttcccaaccttgaaagcattagcaattaagaaatctctaaggcattcataccatgctcttggggcttgcttaagtccatagagcgccttagagagcttacacacgtggtcggcgtaccgttcatcctcgaagccagggggttgctctacgtacacctcctcctttattggcccgttgaggaaagcgctcttcacatccatttggaacaacctgaaagaatggtgagcggcatatgctagcaagatacgaatggactctagcctagccacaggagcaaaagtctcctcaaagtccaaacctgcgacttgggcataaccttttgccacaagtcgagccttgttccttgtcaccactccgtgctcgtcttgtttgttgcggaacacccacttggttcccacaacattttgcttaggacgaggcaccagcgtccaaacttcattcctcttgaagttgttgagctcctcttgcatggccaacacccagtccggatcttgcaaggcctcctctatcctgaaaggctcaatagaagagacaaaggagtaatgctcacaaaaattaactaatcgagatcgagtagttactcccttgctaatgtcacccaaaatttggtcgacgggatgatccctttgaatcatcgcttgaacatgggttggaggtgccggttgcgcatcttcctccatcacatgatcatcttgtgctcccccttgatcacacgcctcctgttgatgaacctgttcatcgtcttgagttgggggtagcaccatagttgaggaaggtggttgatctcgttcatcttgttcctgtggccgcacttctccaatcgccatggttcgtatagcgcccgtcggaacatcttcttcatctacatcatcacaatcaacaacttgctctcttggagagccattagtctcatcaaatacaacgtcgctagagatttcaaccaaacccgatgatttgttgaaaactctatacgcctttgtatttgagtcataacctaacaaaaagccttctacagctttgggagcaaattttgaatttctacccttcttcacaagaatgttgcacttgctcccaaatacacgaaagtacgatacattgggtttgttaccggttagtagctcatatgacgtcttcttgaggaggcggtgaaggtagaccctgttgatggcgtggcaagtcgtgttaacggcttccgaccaaaaacgctcgggggtcttgaattctccaagcatcgtcctcgccatatcgattagcgtcctgttcttcctctctaccacaccattttgctgtggtgtgtagggagcggagaactcgtgcttgatcccttcctcctcaaggaactcctccacttgaaggttcttgaactcggatccgttgtcgctccttatcttcttcaccttgagctcaaactcattttgagctctcctgaggaagcgtttgagggtcccttgggtttcagacttatcctgcaaaaagaacacccaagtgaagcgggaaaagtcatcaacaataactagaccatacttacttcctcctatgcttagataggcgacgggtccgaagaggtccatatgtagcagctccaggggtcttgatgtggtcatcacattcttgctgtgatgtgctcctcccacttgtttacctgcttgacaagctgcacaaggtctatctttttcgaattgcacgttagttaaacctatcacgtgttgtccctttagaagcttgtgaaggttcttcatccccacatgtgctaagcggcgatgccacagccagcccatgctagtcttagctattaagcatgcatctagaccggcctcttcttttgcaaaatcaactaaataaagtttgccgtctaatacacccttaaaagctagtgaaccatcacttcttctaaagacagacacatctacatttgtaaatagacagttataccccatgttgcataattgactaacagatagcaaattatatccaagagactctactaaaaacacattagagatagagtgctcattagaaattgcaattttgcctaacccttttaccttgccttgattcccatcaccgaatataattgaatcttgagaatccttattcttgacgtaggaggtgaacatcttcttctcccccgtcatatggtttgtgcatccgctatcaataatccagcttgaacccccggatgcataaacctgcaagacaaatttaggcttgggacttaggtacccaactcttgttgggtcctataaggttagtcacaatttccttagggacccaaatgcaagttttatcagccttgcattttgcccctaacttcctagcaattacctttctatcctttctacaaattgcaaaggaagcattcaaagcatgatatattgtagaaggctcattaactttcctaggaatattaacaacatttctcctaggcatattatgaacaacatcccttctaccaacatttctatcatgcacatatgaagaactagaagcaaacatagcatgagaaaaatcatcgtacgcattataactcctataagcatttctagtttgtctcctatcatgatacaaaaaggcatgattctttttagcactagtagccataggggccttccctttctccttagcgagaatgggagccttatggcttgttaagttcttggcttccctcttgaagccaagtccatccttaattgaggggtgtctaccgatcgtgtaggcatcccttgcaaattttagtttatcaaattcattcttgctagtcttaagttgggcattaagactagccaattcctcagttaatttggaaattgaaactaaatgatcactacaagcatcaacattgaaatctttacacctagtgcaaatctcaacatgttctacacaagaattagatttactacctacttctagtttagcatttaagtcatcattaacactctttaaagtagcaatggtttcatgacaagaagatagttcactagaaagcacttcatttcttttaacttctaaagcataagatttttgtgcctctacaaatttgtcatgctcttcatacaaaaggtcctcttgtttttctaagagtctatctttttcattcaaggcatcaatcaattcattaattttatctactttagttctatccaatcccttgaacaaactagagtaatctatttcattatcactagactcatcatcactggaagaatcataagtgacattgttttgattacataccttcttctcccttgccataaggcatgtgtgacgctcgtttgggaagagggatgacttgttgaaggcagtggcggcgagtccttcattttcggagtcggaggaggagcaatccgaatcccactcctttccgatatgtgcctcgcccttggccttcttgtaatgcttcttcttttccctcttgctcccgtgttcctggtcactatcattgtcgggacagttagcaataagatgaccaagcttaccgcatttgaagaatgatcgctttcccttggtcttagtcttgcttggctgtcccttgcgaccctttagtgccgtcttgaatcttttgatgatgagggccatctctttatcattaagaccgaccgcctcaatttgcgccaccttgctgggtagcgcctccttgctccttgttgccttgagagcaatgggttgaggctcatggattgggccattcaacgcgtcgtccacgtacctcgcctccttgatcatcattcgcccgcttacaaattttccaagaatttcttcgggcgacatcttgatgtacctaggattttcacgaatattgtttaccaagtgaggatcaagaacggtaaatgaccttagcattaggcggacgacgtcgtgatccgtccatcgcgtgcttccgtagctccttattttgttgataaggatcttgagccggttgtatgtttgtgtcggctcctcgccccttatcatcgcgaatcgtccgagctcgccctccaccaactccatcttggtgagtaaggtgacatcattcccctcatgagagatcttgagggtgtcccaaatttgcttggcattgtccaagccgctcaccttgtgatactcgtccctgcacaaagaggctaacaacacagtagtagcttgtgcatttttatggatctgttcattaataaacgaagggctatccgagctatcaaatttcattccactttccacaatctcccaaatgcttggatggagagaaaataggtgagtacgcattttgtgactccaaaatccgtagtcctctccatcaaagtgaggaggcttgccaagtggaatggggagcaaatgagcatttgagctatgcggaatgcgcgaataatcaaaagaaaagtttgagttaaccgtcttttgtttgtcatagtcgttgtcgtcgttgtccttttgggaagaggaagattcgtcgctgtcgtcgtagtagacgatctccttgatgcgccttgtcttcctcttcttcccttccttccgtctgtggcccgagccggagtcggtaggcttgtcatctttgggctcgttgacgaaggactccttctccttatcgttgatcacgattcccttccccttaggatccctttcttcgggcggttagtccctttcttgaagagaacggctccgataccaattgagagcacctagaggggggtgaataggtgatcctgaataattcaaacttatagccacaaaaacttgttaagcgttagcacagttaatgccaagtggctagagcgaagatcaatatgataatcacaaagagttcaacacagagaagacacagtgatttatcccgtggttcggccaagtacaaaacttgcctactccacgttgtggcgtcccaacggacgagagttgcactcaacccctctcaagtgatccaatgatcaacttgaataccacagtgttatgcttttcttttcttatcgcgtttgtgaggaatctccacaacttggagtctctcacccttacacttgaagttcacaaagaagcacggagtaagggagagaagcaacacacacaaatccacagcaaaatgcgcacacacacgaccaagaatcgagctcaaaagactatctcaaagttctcactagaacggagctcgaatcactgagaatgacaaacgaatgcgcaaagactgagtgtggatgatcaagaatgctctaaggttgcttggtctcttcctccatgcgcctatgggtcccttttatagccccaaggcagctaggagccgttgagagtaaaactggaaggctgatcttgccttctgtcgactggtgcaccggacagtccggtgcacaccggacactgtctggtgcccgatttctttccttaaatggcgcagtcgaccgttgcagatctgggagccgttggcgcaccggacatgtccggtgcacaccggacagtccggtgcccccttccgaccgttggctcggccacgtgtcccgcgcagattgcgcggccgaccgttggcccggccgaccgttggctcaccggacagtccggtgcacaccggacagtccggtgaattatagccgtacgccgtcggcgaaatcccgagagcggccacttcatgccgagtcagcctggcgcaccggacactgtccggtgcaccaccggacagtccggtgtgccagactgagctgagtcttggctgtacacagccaagcttttgcacctctcttcttttctgtttctaacacttagacaaatatattagtacacaaaaccaatttactaaggcttagaaacatacctttactcttgatttgcactttgttcatccatgggcataaattcacatttaagcacttgtgtttgcactcaatcaccaaaatacttagaaatggcccaagagctcatttccctttcacctcctcagcatcttattgtgaatttctgaaaatgctttaaaccccataaacatgatactgtttttcaatggtggtgccgttttttaacgaaatgactcatgtgaatatccaaaaagtagacaagttggaaaaatgatttgaaagtaacaaagatttcatgcatttaattaagaacatttttttgtggcgcgcgctggggggggggggcagtctatttttgctgtcccaattaccaaagtaagccttggaaaatgaactgatcagcgaccagacattgggttggtttccctattattgtaagatttgcagcatgttggttctttacatgcatccttttttggtccctttgcccccccccccctgtgatttatgggtaatttgtcttcgaagaagctgtgctagtaaacagatattcatttatcagcaaatctctagtgtttctttttttttgtgaacttttaagtatattttattgcttgcttacagatccgtgaatatcatcggcggaacccatctgcccgctttgttagtacaactgatgactatgaagagcttctaaaggaggaaccagttattgaattcactggcgaggtattgtcaccttttttgattattgcaacttcgtttgatattttgtgtgcctttttatcatgggtcatatgacgtttaatcttagtaacttgttacttgcacgcatttagtcttagtaacttgttacttgcatgatttttccagatgattgatacttatatgcctgtgactattcaagcctttaaaactattgcagtttatctggacacatctttttgtaattgtttgtgccatgttatagtctttgtgttttgacctatgcaagttacgttgctcatcaacagggttttctaaaaacctttgggcttgtttggtgacaagggaattgaagaggattggaggggattgagagggaaattagttcatttccccctcaatcccctccaatccccctcaattcccttgttaccaaaggagcccttagagtccatcagtgcctttccttttcctttcactacagtagtcttgaagctgagggtatctcctttgtgatacgtgtacttcaatttctgcaggattgtttgattcctcttgtgggatcttggtggtgtcgatggtaagatttgggcaatgattgaaactgcgatgtagaatgatgaataacttgacaatcattcaatgatctattcctctttctgattttcaccgctgcacagtccttttcctgttataccatgttgcatatttactacatttttctgttactattatactattctcacaatagtcttaattaggttttctgatgatctggttctgcattttgataggaaacttttggccgttaccttgacttgcatgagctttacaaacagttttaattatgttttctgatgaagtgatgatctggttctgcattttgataggaaacttttggccgttaccttgacttgcatgagctttataatgagttcataaattccaagttcggaacactgatggaatattcagcttatgttggctgtttctcccagacggacaagatctcacatagtcataaagctaccaggtatgtcatgcatagtttttcactaaggtctgaaatgtacgatctcctggcatgggtttgactagtatggtttacctaccgtaggatcataaggctcaacatgttcaagtctatatgtataatagttgttcaactgtttattgtttaacctgtctagcattcatttcatatatggttaatagggaaaaaccctaaatcatggtatagggtattgcatatccaagtacatgtggatgatagttgctcagctgtttacctgtctaacatttatttcatatgttggtgcccttatatgtcaattataatgttgaatattctttatatcttacaatttctttaattgacagaaaatacagagaatacttggaacatattttggaatatctgacatcatttctgtatcgcacagagccattgcaagacattgataagatttttttaaaggtcagtcttattttaccattttctatattaatgtccatttgtgttccacatgtttatgtgattatgtagaggatcatactttgctttgtattttttaactatgatgtcgcttctttatattcattttggagctgctaatagtgctttggcagtgatttgttcaatagcatagagcatatttatgattaaatacttaataacttacattctaatggcatacattggtacagctggagagtgaattcgaggaacaatgggccaatgaaggaatacttggatgggggaataaagggacagaaaaagagtctgaaatagatctcgattactacagcacagttgaagaacttgttgagcttggcccagaaaaattaaaacaggtttgattttaaaacattatgctgttaccaaaagttattttcttgtctgttattgtcaactgtacataggttcctttctgatgagttctgttgattcatttacatcgttttaggctttagctgctcgaggtttgaagagtggcggtactgttcaacagcgtgcagatcgtcttttcttgttgaaggtgatattaaagacgctagtgctactgctggttcatctattgttcttgcctttttaccattcattttgaactttttgttgattatattttgcaggttacaccgttggaacaactagataggaagcattttgccaaagttccacatactaaagatggctcaaacacagctcctaatggtaatgctttcaaggaggatatgaagaaagaaattgcattgatggaagtgaagatgaaacgtctttgtgagctgctagatgaggtctgctcttcactgctccctgtggttacgacttacgactttactcatgtaattttgaacgcatggtacactcatgcaaatgcactagcatgtgcagtcaatccttttgtgaccacttagggtgcgtttggttacgggacagacaggagagggacgttctctggcgtcctctcctgtccctccaattttgagggataagtggggataacattgggatagtcatgtcccaacccttgaccttgaaccaaaacaaccttatttgagggatcgtcccattccgtcccgtcctgtcattgcaaccaaacacacccttaattcctcatataagctcatcctatgaagatcaaacctgcttactttgaggaatgtactttgttgccatcttaggctagtctattcaaatggaatattgatttctttggctagacctctcgtttgtctggcattacactgttcctcaaattgcaattttgggcatgcagggaagtactaaggtatgattgtagacttgttctgaaatagttcatgtgatgtgtgggtgtttttttccttttgtgtattctctatcactttttgtttgtgttgaaaactccatttatattatcacaacttaagacatgctttaacggctttataaaaaaaattaacagatgattagtgcatcgcctcttcttttttcctaataatactgtgtatgtactggccaccaatataatcacatgccagttctaatatggtagatatgtatatttttgtttgcaaacccaagcacaaactgtaccgggactgtgctacaattccattccagaacaatccacttcttgttactactaaccttctttttgggaagagctactagtgttcgttgcatctttttatgtctatatgttgacattaacctgatatttttgacattttatgttgcttgtgtaggcctttgtaaggacaaaggaaaatgcggagaagaagcttactttgacatatgaagagatggaagcagaacgggaagaggtaattagttacttacccctcttctaaaaaaacttatctgtgtcttgcactattcctggagtccagaatgtgttctttattttcaatgttgtagctgctgctatccttgtgctatcttgaaaacaccaatgagtgtgttttttaacaattctaaaatatgaactgacatatgcatgtctgatactctgatatgctttagaaagagaactactcagctaaagatgctcttggctgcttttttatcagtattgttgctactgagatataccaaaatcatgatcatggacatggaataccattttcctttcccctaaaacagttctgcaagtgtcttggatgtaacccaatcttttttgtattctgaaaatcctttctctgggtcatggcatgctcttttgctttgaaaattttgtagtacatagtgaataagtttccaagttgtttactatgcatttccattgtgatttgctgattagtgttgtcacatttagccctaataacttattaagccgcatatcgtgacaaggttcagctaggcgctaggcggaatctaggcggtgacccattgcctagcgcctagtcgggagtactcggtcttaggcgcttctaggcgcttttctaggcgttttggcaatatagccataaattatatatattatgtatataactatatatacgtatataactactatatatgactatatgagtcacagactcacagtaagtataaaaagaaggccggtagacatatctgattcctagctgagcttactcttgctgtcttgcatgttcctagctcctgcaaggctgcaacacattttgacagctagtagttagtaaattagtcaatagacagctagttgttcataaaaaatagaaaacactaaattgtgacttatctggatgatttcagcagcctcccattcatgagcagttgagcacactgcacaccatatcagcagctggtaattacaacacaagtggataagacagtaatacaagtgcagaacacaatttataaataaaggacaacacaagcacatagttcttagttcaggtctcacacaaaagaagacacacagacacaaagtgacacagctgcagttcataagaccagggcatcacaaaaggcttcactactacatggagatggtccagatcatatttcatcttcactgtagcacaaatcttcatcttcatactcttcttcttcagactcaaactcttctccttcatagagctctctcactcttgcacttctacgaggctcaagctgttcttctgctcccactgcctctccaataacagaccaaggtatccctgtaccttccatctcatcttcctcctcatctctaaagacaacttgtgcacaatcatctccaccgtcttggagaaaaccttgagcttcagttgtatcactagacaagagaacatcagtgattttctttgacttaatcttttctctgttattcatcagcctgttattgaactgaatatagaccaacttgttgaggcgggttgtagtcagcctatttctcttcttagtgtgtatctataaattagaaacagaagcattttcagttctgaaatttaatcatagtactgctgaaatgattaatagataggtcataggtactaaccccttcaaacccactccaatttctttcacaaccagaagagcttgatgtcaaagataatatccttgtagccatcttttgtaaagctggtgtttcagttccatataaccgccaccatgatgctgacataaattagaaacagccaacattagcttcacatccttccaaaaggctggactcaatatagtagctgtggcctcttttcctttctttgatttcacatcctttaatgagtcccacctgctatgaactaccatcttccttaactggtccttcttctcttgcatactgttcaaagtgagaaagtatgaagcaaacctagtcactcctggcctcactagctctttcccctctgtgaagtatctcaagcactccaatgttcttgtgtggccatacacaaatatggtaaatgactttgcttggtcaatcactttcctgaaccgaggcaatttgccaattccttggagcatcaagttgattgtgtgagctgcacaagaggtccaaaatatttgtggtctcttctcaagcaatagcttctttgctcccatgttgttagaggcattgtcagtgactacttgcaccacattttcttcaccaatgtcttcaattgctttgtccactaattcaaaaatgacttcgcttgtgtgtgacacatctgacatctcttttgagctgatgaaggaggttccatcagcacaattagtgcatatattcattatgcttctcctcttcctatctgaccaagcatcggtcataatagagcaaccattttttcatcttctcggcttcacgttcctgcagcaaacacttggttctttcatatttcttcttccagcaaactacctcgaaaggcatcttgagttggaggtgtaagtcctggtccaaattgtccaattgcttcacacatttgcttgaactcatcattgtcacatgcattgaaaggtattcctgccaatgattaaaaaatgaagtctaattttaaaatgtgttcagttcaaagttcatgaattttaaaatgtgaaaacagcatcgtatactagcaatttaccatgattataggcccatcttgcaataaacttgtgcacctcatgctctctttctttccatagttccttgttcagctgctgttgtttgaatgaatcagccttggtaggatcaatagcacgtgtccatttgtcaattggccctaatttgtgaggctgtgagcttccaacacaagtgacttcttctgactcctctccaaccctagatacattcacttcctctctaagttctagctcacgaacagtcttctcctccctcttcctttttgcagcctctattgctttcttgcacttctctttagcctctagagcctgcggtgttgcagacgtgcatttcttcacattctttccaacatgggcaagatgttccttcaacctataaatccctcccctcatctccttgtcacagaacttacacttcaccttgtctttgttgttagcatcaacaagaacaccatattcccatccaacatcatctgaatttctttttaggagattcgctctagctgcttcagtttcagaaggtgtagctgcagtttctgatgacatccttaaaccttaatcctttgatttctttcacttgtacactgcacaggggaggaaagcagtttcagcaggggaggaaagcaggggaggaaagcaggaaagcaggggaggaaagcaggggagggggatgagcagggaggggagcagccgacgggggaggagaactcaccggcgggggagcagggagccgacgggggagcagcaggaggggatctcaccggcgggggagcagggcgccggcggcggagccggcggtggagcagggagccgggcggtggagcagggagccggcagggagactgcgctaaaactgccctgcgcgctaaaactcacgcgcgctaaaaactaacgcgcgctgcgcgcgctaaaacgcgcgctctcccgcctgcgcgcccgcatgcgcgcccgcgcgcccgtcctgcgcgcccgcctgcgcgcccgcttaggcgctgtgcgccgcccactcgccgcctaggcgccgcggccgcctagaagacgcacagggcgctagtctacgcggcaggctgccgactagcgcctaggcgggcctaatcgccgcctagtcggcgcctagctgaacactgtatcgtgacgagtccatggcaaccactgctttctggaatatgcaggagagctgcacatctttgtattaatagaagaagaaaggggagcggaccccattacatcacacacattacaacactagaaactaacatatgatcaccgactaacactgagagaacacgacccacaacgctgttttaacccagcaactcttcaacctggaggaaagacagggccttggctcgagtcaaggacccttggctccagtcaaggaccagagcaccgcctcttccctagccaccctaaaggctgtgaccaagctaggattacatccatcaaagacacgtctattcctatgcttccatatggtccaagcacctaagataactaaagaattgaaaccctccctcaaatgatcagatgccagagagctgctagtcttccaccacgactcgaaggtaatgactgttgtgcaagctgtccttaggtgcaagcgtgcaagtgactcttctagtccgttagatcatgatccaaccatagatgcaattgtggtttgttagttaaaaaaaacacaacccgatggtgggagggtttaaatgttaaatgtgacatacggttggatcatgatataacggatcaggaggctcccttgcacacttacacctaaggacagcttgcacaacagtagttgccgtcacatttagctctaataacttattaagccgcatttcgtgacgagtccaatgtaaaatcttagtggatagtggtagtagcttgttgtaaaggtgaacaaatgttttcagttagggacttataaggctttggcttgtttgcttcaaattcattgtatgatcttataccacttggctgaaaatagtatttttcatgcatggaagtaaactacttagtttttcaactactggcttatatatatgtattttctataggaggaggtacaagttgacactgagagtgatgacgaagagcaacaaatctacaatcctctcaagttgccgatgggttgggatggcaaacctatcccctactggctgtacaagctacatggccttgggcaggtaactagttgtcatatttgggagtatatttcctagtagcttgttacctctagtcttagtcagtgagatggcacggcagtagcaccaatgctttcactcatggtgcagattgactagttggcacccattaagatgccagtcatttatgaactgatcagtcattacttcacatctatgacaccaaaaatgaaattctcttatgctatataaattgggtacccatccttgttcagtgactaatacttatgcaagtcagtaaggatgttagcagtataggatgaatatgaagtcttctattaagttttctgcagtgttagcattcaatagtaaccttttggagtttaatttctagggactttccacctaatgaggtgattgttttctcctcacccagtctagagcattaaaagattctatttctgcataaactgtatctgtatgcaaatctagaaatgtgatccatctgactaatgtatttatctgaacacatgttcacgtttgttagtgcattttctctcttcaggaagtgctttgaaccagaaaatcaaggcacctttttcttttatgcttagtaagctctagcaaatttgttttctgatgcatttttaactgttatggacttatgctttgttcaggaattcaagtgtgagatatgtggaaaccatagctattgggggcgaagggcttatgagcgtcatttcaaggaatggcgtcatcagcatgggatgcgatgccttggcattcccaatactaagaatttcaatgaaattacatccatcgaggtatatcatctcttgttttattgatattattttatttacttggtatacttattggtgcatagcttactacaatgtttatttactttttactagtataatactaatacccgtgcgttggaacggtacacaatcgtatttcatgatattggttgggaagggcgaggctgtagccgcaatgcaaccaatggttgaacgaagtaaaccagtcatatggatcctccctaaaaaataccaataataaataaatttaatatcaaagtgagcatattgtctatagacaagataataaaaataaatattatactttattttagccaaaaagaccgagaaagatgagttgaaaaagaactcaatccctatttttatagagctctctctagctcgtcctccttcaactaacgggctgttactatatgtgtacggcttcctgtcgtattgcgcttggtggcttctcacggtctatatatggtgtaacaaccttgatgacttataactcaagacattgactccatataaacacacattatggctgccggttttaaagacccacaacatcttccactgaacctatgcccttttattttctcttagacacacgcattaaacacacaattcacggacagatagaatcataaagatgtctctagctattgcacaataatctgatagagaaggaaaacatgcctcaacaattatcaaacagaggtcatgcatggattcatgatgcaacatgcatgaaccatgcatgagttgatcggtgtccagtatcgtcttgcgtcatgcacgaggggtggttaattctgatcaagctacaagaaacaaatgaactaatgaatcaaatgccttgaacaacagccatccactacgaggggtggttaattctgatcaagctacaagaaacaaatgaactaatgaaccaaatgccttgaacaacagccatccactacgtatctgtcgtccatgctttgcatcggaaggccattcccaactgtcctagctctagcaaaacagtacacactcaacttcgaatggagctacaataaaatgctcgccaaggaaccatcgcaaacacacgacgaccttgtagctcctcactcctgaggaagcacttgtcctagccgcttgcaatgacttgttacgcttcgtctctcttctccgccacaacggtcgtgaccacgttgtgcggcagcactacatgccacgttgtgcggcatcttgctcacgccccgtcgctcttgtcgccgtcgatcgtgcccgtggccacccttccaccgatgctcgccataccaacggttcccgcggtgggcgccgtgccacaggtccatgtggtgccgaataccgccgtcatgccaaaagggacactgccgccaatcctcgccgtcgtgtcgaaggttggcgctttgatgccaatgcctgtcgtcgtgcctaaggttggtgagttgccgttgatgccctctctgtggcccgtgcctaaccgctcatgccctccatcccgacagcatcggtctccattaggtcccacgaacgtgacacagactcggtggacaggcctgtgaagatgcttgtggagtcaatgagccagaaaatcagatgtacgtacgtgcgtaccttggaggacgtaggcaagtcggactagctgaagagcgtcaggccaccggtggcgagcaaatccaccaccgctatcgtttgagggtcctcaatatgacttttcgtagccaggttggtgggcggattaggatgagtaaaacggctctaccccgcagcttctggaaccactttccccgcgtttctatgcatggaagacgcagcgtgtgtgggtctgctgaaaccgctttccctatcccctacggagcgacgtgcatgggttgttgggctcgcggtgcatttggcgcgggctcacttgtcatgccgatcgttgctatgaacagaagacatgacatgtgcgggtttctgtcatttcgtgggtgactgaggcattcgtgcgtcgtgcggggcgctgattcgctgaggacgagtggatagacgaattggattagatgcgattgatgtgaagagaatgtatcttgtgggtcatttcaaacaatagaagtagttatttgtaaaaatatgacgcgggacgaccgtcgaaacttgtgctttaatatatatatatatatatatatatatatatatatatatatatatatatatattattgcaaaggagacgatggatcatgatctatttcaaagctaaggtatatgatatattccgtaattgtaatagcagaaaaaacgaccggaaaagacacttgttttctctgtgggccatgactccgtcacgtgaagaatctgaaccgtggtgagatttggtgacggggacgcatggcgccaagccgacctcgcgactgccggcaggtttggcggatttgtctgcacaaaagcgtcgctgcttcctatgtttacgtctagcgatccagcctcaagctagtttgacagtggtcaaagtcggcgaggggtcatttcaaacaacataaggtggtatttgtaaaaatatgacgtgggacgatcgttgaaactggtgctttaatatagtagagatgagatttgcccatatcatattggcttccacatttccttgtcctttatttattctgtttccttatttgaaacagaatgcaaaagaatgcatgtctgccatattcttgacattggccggtttattgtatattgtatgtgctttaacatttatttctactctcaggaggcgaaagcgctctgggagaaaattcaagcacgacaaggggtgaataagtggcggccagacctagaggaagagtatgaagatcaggaaggcaacatctacaacaagaagacctacactgacctgcagcgtcaaggcctgatctagggctcctgctggttaaagttgtcgggatttgttcagaacttatctcatgtagttgtaactctgaaaatattggcccatctggcatacattttatgtaataacatgattctccggaactcatgtgcttttcccggttgtcatggttgtctcgtgtgtctgtttgcccagcttggtcgtccgtagctatgttatgttttatataatcgtaaatgtgttattgtattaagcacaagttggtgtttgcatcacgtaaaacatatttttataaaaaaattagaaaaaaataaaactaggctgtttttggtcta

SEQ ID NO: 27 - DNA, 1515 bp, Zea mays
  source 1..1515: mol_type = genomic DNA
atggcgtcaacggtgctggaggcgacgcgggcggcccatgaggatctggagaggctggagcggctcgcggtgagcgagctgcaacgggagcccgctaacccgcgtgaccgcctctttcagtcccaccgcgtccgccacatgctcgacctcgtcgtctccacctccggcaagctcgtggaaatttacgaggacaaggacaatgctaggaaggatgagatcaacactcatcttactgcacccacgcaaagtggactgtttagcaagtactatgaaaggctcaaagagatccgtgaatatcatcggcggaacccatctgcccgctttgttagtacaactgatgactatgaagagcttctaaaggaggaaccagttattgaattcactggcgaggaaacttttggccgttaccttgacttgcatgagctttataatgagttcataaattccaagttcggaacactgatggaatattcagcttatgttggctgtttctcccagacggacaagatctcacatagtcataaagctaccagaaaatacagagaatacttggaacatattttggaatatctgacatcatttctgtatcgcacagagccattgcaagacattgataagatttttttaaagctggagagtgaattcgaggaacaatgggccaatgaaggaatacttggatgggggaataaagggacagaaaaagagtctgaaatagatctcgattactacagcacagttgaagaacttgttgagcttggcccagaaaaattaaaacaggctttagctgctcgaggtttgaagagtggcggtactgttcaacagcgtgcagatcgtcttttcttgttgaaggttacaccgttggaacaactagataggaagcattttgccaaagttccacatactaaagatggctcaaacacagctcctaatggtaatgctttcaaggaggatatgaagaaagaaattgcattgatggaagtgaagatgaaacgtctttgtgagctgctagatgaggcctttgtaaggacaaaggaaaatgcggagaagaagcttactttgacatatgaagagatggaagcagaacgggaagaggaggaggtacaagttgacactgagagtgatgacgaagagcaacaaatctacaatcctctcaagttgccgatgggttgggatggcaaacctatcccctactggctgtacaagctacatggccttgggcaggaattcaagtgtgagatatgtggaaaccatagctattgggggcgaagggcttatgagcgtcatttcaaggaatggcgtcatcagcatgggatgcgatgccttggcattcccaatactaagaatttcaatgaaattacatccatcgaggaggcgaaagcgctctgggagaaaattcaagcacgacaaggggtgaataagtggcggccagacctagaggaagagtatgaagatcaggaaggcaacatctacaacaagaagacctacactgacctgcagcgtcaaggcctgatctag

SEQ ID NO: 28 - protein, 504 aa, Zea mays
MASTVLEATRAAHEDLERLERLAVSELQREPANPRDRLFQSHRVRHMLDLVVSTSGKLVEIYEDKDNARKDEINTHLTAPTQSGLFSKYYERLKEIREYHRRNPSARFVSTTDDYEELLKEEPVIEFTGEETFGRYLDLHELYNEFINSKFGTLMEYSAYVGCFSQTDKISHSHKATRKYREYLEHILEYLTSFLYRTEPLQDIDKIFLKLESEFEEQWANEGILGWGNKGTEKESEIDLDYYSTVEELVELGPEKLKQALAARGLKSGGTVQQRADRLFLLKVTPLEQLDRKHFAKVPHTKDGSNTAPNGNAFKEDMKKEIALMEVKMKRLCELLDEAFVRTKENAEKKLTLTYEEMEAEREEEEVQVDTESDDEEQQIYNPLKLPMGWDGKPIPYWLYKLHGLGQEFKCEICGNHSYWGRRAYERHFKEWRHQHGMRCLGIPNTKNFNEITSIEEAKALWEKIQARQGVNKWRPDLEEEYEDQEGNIYNKKTYTDLQRQGLI

SEQ ID NO: 29 - DNA, 1479 bp, Triticum aestivum
  source 1..1479: mol_type = genomic DNA
atggcgtcgtctgtgctggaggccacgcgggcggcgcacgaggacctcgagcggttggagcgcctggcggtgcgcgagctccagcgcgacccggccaacgcccgcgaccgcctcttccagtcccaccgcgtccgtcacatgctcgatctcgtcgtctccacgtccgacaagctggtggaaatctatgaagacaaggacggtgctaggaaggatgagatctccacccatcttaccgcgccggtgcaaagtgacatcttccccaagtactatgaaaggctgaaagagattcgtgattaccatagacggaatcactctgcccgtttcgtcagtgaaactgatgattatgaggagctactaaaggaggaaccagctattgaattcactggcgaggaagcatttggccggtacttggacctacatgagctctacaacgagttcataaattccaagtttggatcactaatggaatactcagcatacgttggcacttttgctcagactgaaaaaatcgcacataatctaaaagctaccaggccttacaaagaatatttggagcatattttggaatatctgatgtcatttctgtatcgtacagaaccgttgcaagatattgagaagatttttacaaagttggaaagtgagtttgaagaacagtggaccaatggcgaagttcctggatgggagaataagggcacagagaaagaatctgtgttgcaagagtctgcagtagaccttgattactacagtactgttgaagagcttgtagagcttggtccggaaaagttgaaggaggctttaactgctcgtggattgaagggtggcggcactgttcaacagcgtgctgagcggcttttcttgctgaagcatacacccttggaaaaactggatcgtaagcattttgccaaaggtgatgatttgaagaaggaaattgctttgactgaaatgaagatgaagcgcttatgtgagattctcgatgaggtcattgtaagaacaaaggaaaacgcggagaagaagctgaccttgacctatgaagaaatggaagcagagcgggaagaggaagaagtgcaagctgatagtgaaagtgatgatgaagaccaacaaatctacaaccccctcaagttaccaatgggctgggacgggaaacctatcccttattggctctataagcttcacggtcttggtcaggaattcaagtgtgaaatatgtggtaaccacagttactgggggcgaagggcttatgagcgccatttcaaggagtggcgccatcagcatgggatgcgatgccttggcattcctaatactaagaacttcaatgaaattacatccatcaaggaggcgacgacactctgggagagaatacaagcaaagcaagggcagaacaagtggcggccagacttggaagaagagtatgaagataaggatggaaacatctacaacaaaaagacctatactgatctgcagcgccaaggcctgatatag

SEQ ID NO: 30 - protein, 492 aa, Triticum aestivum
MASSVLEATRAAHEDLERLERLAVRELQRDPANARDRLFQSHRVRHMLDLVVSTSDKLVEIYEDKDGARKDEISTHLTAPVQSDIFPKYYERLKEIRDYHRRNHSARFVSETDDYEELLKEEPAIEFTGEEAFGRYLDLHELYNEFINSKFGSLMEYSAYVGTFAQTEKIAHNLKATRPYKEYLEHILEYLMSFLYRTEPLQDIEKIFTKLESEFEEQWTNGEVPGWENKGTEKESVLQESAVDLDYYSTVEELVELGPEKLKEALTARGLKGGGTVQQRAERLFLLKHTPLEKLDRKHFAKGDDLKKEIALTEMKMKRLCEILDEVIVRTKENAEKKLTLTYEEMEAEREEEEVQADSESDDEDQQIYNPLKLPMGWDGKPIPYWLYKLHGLGQEFKCEICGNHSYWGRRAYERHFKEWRHQHGMRCLGIPNTKNFNEITSIKEATTLWERIQAKQGQNKWRPDLEEEYEDKDGNIYNKKTYTDLQRQGLI

SEQ ID NO: 31 - DNA, 1479 bp, Triticum aestivum
  source 1..1479: mol_type = genomic DNA
atggcgtcgtctgtgctggaggccacgcgggcggcgcacgaggacctcgagcggttagagcgcctggcggtgcgcgagctgcagcgcgaccccgccaacgcgcgcgaccgcctcttccagtcccaccgcgtccgccacatgctcgatctcgtcgtctccacttccgacaagctggtggaaatctatgaagacaaggacggtgctaggaaggatgagatctccacccatcttaccgcgccggtgcaaagtgacatcttccccaagtactatgaaaggctgaaagagattcgtgattaccatagacggaatcactctgcccgtttcgtcagtgaaactgatgattatgaggagctactaaaggaggaaccagctattgaattcactggcgaggaagcatttggccggtacttggacctacatgagctctacaacgagttcataaattccaagtttggatcactaatggaatactcggcatacgttggcacttttgctcagactgaaaaaatcgcacataatctaaaagctaccaggccatacaaagaatatttggagcatattttggaatatctgatgtcgtttctgtatcgtacagaaccgttgcaagatattgagaagatttttacaaagttggaaagtgagtttgaagaacagtggaccaatggcgaagttcctggatgggagaataagggcacagagaaagaatctgtgttgcaagagtctgcagtagaccttgattactatagtactgttgaagagcttgtagagcttggtccggaaaagttgaaggaggctttaactgctcgtgggttgaagggtggcggcactgttcaacagcgtgctgagcgacttttcttgctgaagcatacacccttggaaaaactggatcgcaagcattttgccaaaggtgatgatctgaagaaggaaattgctttgattgaaatgaagatgaagcgcttatgtgagattctcgatgaggtcattgtaagaacaaaggaaaacgcggagaagaagctgaccttgacctatgaagaaatggaagcagagcgggaagaggaagaagtacaagctgatagtgaaagtgatgatgaagaccaacaaatctacaaccccctcaagttaccaatgggctgggacgggaaacctatcccttattggctctataagcttcacggtcttggtcaggaattcaagtgtgaaatatgtggtaaccacagttactgggggcgaagagcttatgagcgccatttcaaggagtggcgccatcagcatgggatgcgatgccttggcattcctaatactaagaatttcaatgaaattacatccatcaaggaggcgacggcactctgggagagaatacaagcaaagcaagggcagaacaagtggcggccagacttggaagaagagtacgaagataaggatggaaacatctacaacaaaaagacctatactgatctgcagcgccaaggcctgatatag

SEQ ID NO: 32 - protein, 492 aa, Triticum aestivum
MASSVLEATRAAHEDLERLERLAVRELQRDPANARDRLFQSHRVRHMLDLVVSTSDKLVEIYEDKDGARKDEISTHLTAPVQSDIFPKYYERLKEIRDYHRRNHSARFVSETDDYEELLKEEPAIEFTGEEAFGRYLDLHELYNEFINSKFGSLMEYSAYVGTFAQTEKIAHNLKATRPYKEYLEHILEYLMSFLYRTEPLQDIEKIFTKLESEFEEQWTNGEVPGWENKGTEKESVLQESAVDLDYYSTVEELVELGPEKLKEALTARGLKGGGTVQQRAERLFLLKHTPLEKLDRKHFAKGDDLKKEIALIEMKMKRLCEILDEVIVRTKENAEKKLTLTYEEMEAEREEEEVQADSESDDEDQQIYNPLKLPMGWDGKPIPYWLYKLHGLGQEFKCEICGNHSYWGRRAYERHFKEWRHQHGMRCLGIPNTKNFNEITSIKEATALWERIQAKQGQNKWRPDLEEEYEDKDGNIYNKKTYTDLQRQGLI

SEQ ID NO: 33 - DNA, 1479 bp, Triticum aestivum
  source 1..1479: mol_type = genomic DNA
atggcgtcgtctgtgctggaggccacgcgggcggcgcacgaggacctcgagcggttagagcgcctggcggtgcgcgagctgcagcgcgaccccgccaacgcgcgcgaccgcctcttccagtcccaccgcgtccgccacatgctcgatctcgtcgtctccacttccgacaagctggtggaaatctatgaagacaaggacggtgctaggaaggatgagatctccacccatcttaccgcgccggtgcaaagtgacatcttccccaagtactatgaaaggctgaaagagattcgtgattaccatagacggaatcactctgcccgtttcgtcagtgaaactgatgattatgaggagctactaaaggaggaaccagctattgaattcactggcgaggaagcatttggccggtacttggacctacatgagctctacaacgagttcataaattccaagtttggatcactaatggaatactcagcatacgttggcacttttgctcagactgaaaaaatcgcacataatctaaaagctaccaggccatacaaagaatatttggagcatattttggaatatctgatgtcatttctgtatcgtacagaaccgttgcaagatattgagaagatttttacaaagttggaaagtgagtttgaagaacagtggatcaatggcgaagttcctggatgggagaataagggcacagagaaagaatctgtgttgcaagagtctgcagtagaccttgattactacagtactgttgaagagcttgtagagcttggtccggaaaagttgaaggaggctttaactgctcgtggattgaagggtggtggcactgttcaacagcgtgctgagcgacttttcttgctgaagcatacacccttggaaaagctggatcgtaagcattttgccaaaggtgatgatttgaagaaggaaattgctttgattgaaatgaagatgaagcgcttatgtgagattctcgatgaggtcattgtaagaacaaaggaaaatgcggagaagaagctgaccttgacctatgaagaaatggaagcagagcgggaagaggaagaagtacaagctgatagtgaaagtgatgatgaagaccaacaaatctacaaccccctcaagttaccaatgggctgggacgggaaacctatcccttattggctctataagcttcacggtcttggtcaggaattcaagtgtgaaatatgtggtaaccacagttactgggggcgaagggcttatgagcgccatttcaaggagtggcgccatcagcatgggatgcgatgccttggcattcctaatactaagaacttcaatgaaattacatccatcaaggaagcgacagcactctgggagagaatacaagcaaagcaagggcagaacaagtggcggccagacttggaagaagagtatgaagataaggatggaaacatctacaacaaaaagacctatactgatctgcagcgccaaggcctgatatag

SEQ ID NO: 34 - protein, 492 aa, Triticum aestivum
MASSVLEATRAAHEDLERLERLAVRELQRDPANARDRLFQSHRVRHMLDLVVSTSDKLVEIYEDKDGARKDEISTHLTAPVQSDIFPKYYERLKEIRDYHRRNHSARFVSETDDYEELLKEEPAIEFTGEEAFGRYLDLHELYNEFINSKFGSLMEYSAYVGTFAQTEKIAHNLKATRPYKEYLEHILEYLMSFLYRTEPLQDIEKIFTKLESEFEEQWINGEVPGWENKGTEKESVLQESAVDLDYYSTVEELVELGPEKLKEALTARGLKGGGTVQQRAERLFLLKHTPLEKLDRKHFAKGDDLKKEIALIEMKMKRLCEILDEVIVRTKENAEKKLTLTYEEMEAEREEEEVQADSESDDEDQQIYNPLKLPMGWDGKPIPYWLYKLHGLGQEFKCEICGNHSYWGRRAYERHFKEWRHQHGMRCLGIPNTKNFNEITSIKEATALWERIQAKQGQNKWRPDLEEEYEDKDGNIYNKKTYTDLQRQGLI

SEQ ID NO: 35 - DNA, 3518 bp, Zea mays
  source 1..3518: mol_type = genomic DNA
ctgcaccaacaggtcccgccagcaccaacccaacgcaacccagcgtcccgtcgcaacgatacaatagaagccgcacgaaccccgcgctgcgctgttctggagaggtcccgtgcgcgcccgaaccctccgccggttgcgctcatgctcacgcctacgacgtcgcccgcgcgccgggccgcgctcgcaccgccacatcaccgcactacacgaaccttcccgaacgccaccgctccccgccctacgcctgtatttaagcggacgcacctatccgcctcactccgatcaactcaggtagcagagcgaagcatcaatcagcgagcgaaccggccagaaaaagcacaaagtagtcctcctccatttcgaatcgcgctcatagtcacagggctttagcagagatggcgggcaacaagggagagggcccggcgatcggcatcgacctcggcacgacctactcctgcgtcggggtctggcagcatgaccgggtggagattatcgccaacgaccagggcaaccgcaccacgccgtcgtacgtggcgttcaccgacaccgagaggctcatcggcgacgccgccaagaaccaggtcgccatgaatcccaccaacaccgtcttcggtatgcccatgttcttcccctgaataattctagttccatagtttcagtttcctcttatttaatattttagaaataaaaacaatcgccacggctttcgctgatacttgcctctcagcagtcattgagtcatgctctgctcccactctatttttgcggcagctcatgaatcacttgttgcatgttctagatatatctagatcacaatttcacaataattttgagttgtaaaacctttttttttttgctgtttatcgtttcaattacattagtttgctatgtaagtttgtcttgaaatagcaactcctttttttttacctttgttcattcaacagatcattacaaaatatttataggatttttagttgtaaaatattaatagtaccctttacatgtatgtcagatgattattttttgttattgtttatgaaagggttatttgatcatcgtttagctatagaaaaatcatgaatcatctttagttaattttctttttactgtactagtcactggtgtaagattattgtctgacgtggtgtaagattattgtctgaattttcagaccactgagatactgcaactcctacactattacactcataatttttttctccaaatgggaggaaagtagcacagaaatcggtaattatccatcaactgtattgcctaaccgctctgtgtttcttccatgaagatgccaagcgactcatcggccggcgcttctcggacccgtctgtgcaggcggacatgaagatgtggccattcaaggtcgtcccaggccccgccgacaagccaatgatcgtggtgacttacaagggcgaggagaagaagttctcggccgaggagatctcctccatggtgctcaccaagatgaaggagatcgccgaggcctacctcagcaccaccatcaagaacgccgtcattaccgtgccggcctacttcaatgactcgcagcgccaggccaccaaggacgccggcgtcattgccggcctcaacgtcacgcgcatcatcaacgagcccacggccgccgccatcgcctacggactcgacaagaaggccaccagcactggcgagaagaacgtgctcatcttcgaccttggcggtggcacctttgatgtgtccatcctcaccattgaggagggcatcttcgaggtcaaggccacggctggtgacacccacctgggaggcgaggacttcgacaaccgcctggtgaaccacttcgtgatggagttcaagaggaaacacaagaaggacatcagcggcaacccgagggcgctccggcggctgcgtaccgcgtgcgagagggcgaagaggacgctctcctccaccgcccagaccaccattgagatcgactcgctctacgagggcatcgacttctacgcgaccatcacccgggccaggttcgaggagcttaacatggacctcttcaggaagtgcatggagcccgtggagaagtgcctccgcgacgccaagatggacaagtcacagatccacgacgtcgtgctcgtcggaggttccacccgtatccccaaggtgcagcagctgctccaggacttcttcaacggcaaggagttgtgcaagagcatcaaccctgacgaggccgtcgcgtacggcgccgctgtccaggccgccatcctcagcggcgagggcaaccagaaggtgcaggacctgctccttctcgatgtcacgccgctctcgctcgggctggagactgcaggcggtgtcatgaccgtgctgatcccgaggaacaccaccatccccaccaagaaggagcaggtgttctccacctactccgacaaccagcccggcgtgctgatccaggtgtacgagggtgagaggacgaggaccaaggacaacaacctgctcggcaagttcgagctgaccggcatcccgccggcgccacggggcgtgccccagatcaacgtgaccttcgacatcgacgcgaacggcatcctgaacgtgtcggcggaggacaagacgacggggaagaagaacaagatcaccatcaccaacgacaagggccggctgagcaaggaagagatcgagcgcatggtgcaggaggcggagaagtacaagaccgaggacgaggaggtgaagcgcaaggtggaggcccgcaacgcgctggagaactacgcgtataacatgcgcaacacggtgcgggacgagaagatcgcgtcgaagctgcccgccgatgacaagaagaagatcgaggacacgatcgaggacgccatcaagtggctcgacggcaaccagctcgcggaggccgaggagttcgaggacaagatgaaggagctggagagcatctgcaaccccatcatctcacagatgtaccagggcggcgcgggcgctgcgggcatggacgaggatgtgcccggcggtggcgccggcaacggaggtggcagcggcgccgggcccaagattgaggaggtcgactgagcgagccatggacggtgatgatctggatgggaagcttctgtgcgtgcggtgcagctagagagtgtgctacgtgaactcgtgaaggcgtattaatctgtgtgtgcgataataatctggggttaacacctgcagggtgttctctgtttccgttgtcaagtggttgtaggttcagtagggtgaaagtgtgaggcatatccattaggcttagtaaaatagtaaccctagtgttactgtaaccgcctccgcgagttcaggagtctataaatatatcagtatgtaatactgtttgtttttctcgctatgcacgttctagcatccaatcatcccatacctctgccattcctgaacaccccgtgtgcttcagagtatccaaaatccgccagagccagagtcaactcgttggtgatgaaatgtgtgcacacaccagcgcactggaagacaagcaaaaagtccttcagtatatgttttgggac

SEQ ID NO: 36 - DNA, 1959 bp, Zea mays
  source 1..1959: mol_type = genomic DNA
atggcgggcaacaagggagagggcccggcgatcggcatcgacctcggcacgacctactcctgcgtcggggtctggcagcatgaccgggtggagattatcgccaacgaccagggcaaccgcaccacgccgtcgtacgtggcgttcaccgacaccgagaggctcatcggcgacgccgccaagaaccaggtcgccatgaatcccaccaacaccgtcttcgatgccaagcgactcatcggccggcgcttctcggacccgtctgtgcaggcggacatgaagatgtggccattcaaggtcgtcccaggccccgccgacaagccaatgatcgtggtgacttacaagggcgaggagaagaagttctcggccgaggagatctcctccatggtgctcaccaagatgaaggagatcgccgaggcctacctcagcaccaccatcaagaacgccgtcattaccgtgccggcctacttcaatgactcgcagcgccaggccaccaaggacgccggcgtcattgccggcctcaacgtcacgcgcatcatcaacgagcccacggccgccgccatcgcctacggactcgacaagaaggccaccagcactggcgagaagaacgtgctcatcttcgaccttggcggtggcacctttgatgtgtccatcctcaccattgaggagggcatcttcgaggtcaaggccacggctggtgacacccacctgggaggcgaggacttcgacaaccgcctggtgaaccacttcgtgatggagttcaagaggaaacacaagaaggacatcagcggcaacccgagggcgctccggcggctgcgtaccgcgtgcgagagggcgaagaggacgctctcctccaccgcccagaccaccattgagatcgactcgctctacgagggcatcgacttctacgcgaccatcacccgggccaggttcgaggagcttaacatggacctcttcaggaagtgcatggagcccgtggagaagtgcctccgcgacgccaagatggacaagtcacagatccacgacgtcgtgctcgtcggaggttccacccgtatccccaaggtgcagcagctgctccaggacttcttcaacggcaaggagttgtgcaagagcatcaaccctgacgaggccgtcgcgtacggcgccgctgtccaggccgccatcctcagcggcgagggcaaccagaaggtgcaggacctgctccttctcgatgtcacgccgctctcgctcgggctggagactgcaggcggtgtcatgaccgtgctgatcccgaggaacaccaccatccccaccaagaaggagcaggtgttctccacctactccgacaaccagcccggcgtgctgatccaggtgtacgagggtgagaggacgaggaccaaggacaacaacctgctcggcaagttcgagctgaccggcatcccgccggcgccacggggcgtgccccagatcaacgtgaccttcgacatcgacgcgaacggcatcctgaacgtgtcggcggaggacaagacgacggggaagaagaacaagatcaccatcaccaacgacaagggccggctgagcaaggaagagatcgagcgcatggtgcaggaggcggagaagtacaagaccgaggacgaggaggtgaagcgcaaggtggaggcccgcaacgcgctggagaactacgcgtataacatgcgcaacacggtgcgggacgagaagatcgcgtcgaagctgcccgccgatgacaagaagaagatcgaggacacgatcgaggacgccatcaagtggctcgacggcaaccagctcgcggaggccgaggagttcgaggacaagatgaaggagctggagagcatctgcaaccccatcatctcacagatgtaccagggcggcgcgggcgctgcgggcatggacgaggatgtgcccggcggtggcgccggcaacggaggtggcagcggcgccgggcccaagattgaggaggtcgactga

SEQ ID NO: 37 - protein, 652 aa, Zea mays
MAGNKGEGPAIGIDLGTTYSCVGVWQHDRVEIIANDQGNRTTPSYVAFTDTERLIGDAAKNQVAMNPTNTVFDAKRLIGRRFSDPSVQADMKMWPFKVVPGPADKPMIVVTYKGEEKKFSAEEISSMVLTKMKEIAEAYLSTTIKNAVITVPAYFNDSQRQATKDAGVIAGLNVTRIINEPTAAAIAYGLDKKATSTGEKNVLIFDLGGGTFDVSILTIEEGIFEVKATAGDTHLGGEDFDNRLVNHFVMEFKRKHKKDISGNPRALRRLRTACERAKRTLSSTAQTTIEIDSLYEGIDFYATITRARFEELNMDLFRKCMEPVEKCLRDAKMDKSQIHDVVLVGGSTRIPKVQQLLQDFFNGKELCKSINPDEAVAYGAAVQAAILSGEGNQKVQDLLLLDVTPLSLGLETAGGVMTVLIPRNTTIPTKKEQVFSTYSDNQPGVLIQVYEGERTRTKDNNLLGKFELTGIPPAPRGVPQINVTFDIDANGILNVSAEDKTTGKKNKITITNDKGRLSKEEIERMVQEAEKYKTEDEEVKRKVEARNALENYAYNMRNTVRDEKIASKLPADDKKKIEDTIEDAIKWLDGNQLAEAEEFEDKMKELESICNPIISQMYQGGAGAAGMDEDVPGGGAGNGGGSGAGPKIEEVD

SEQ ID NO: 38 - DNA, 1974 bp, Triticum aestivum
  source 1..1974: mol_type = genomic DNA
atggcgagcaagggaggcaacaagggggagggccctgccatcggcatcgacctcggcaccacctactcctgcgtcggcgtctggcagcatgatcgggtggagatcgtcgccaacgaccagggcaaccgcaccacgccgtcctacgtcgccttcaccgacaccgagcggctcatcggcgacgccgccaagaaccaggtcgccatgaaccccaccaacaccgtttttgatgccaagcgactcatcggacggcgcttctcggacgcatccgtacagtcggacatgaagatgtggccgttcaaggtggtccccggcgccggcgacaagccgatgatcgtggtcacctacaagggggaggagaagaccttctctgctgaggagatatcctccatggtgctcaccaagatgagggagatcgccgaggccttcctcagcacgaccatcaacaacgccgtcgtcaccgtcccggcctacttcaacgactcccagcgccaggccaccaaggacgccggcgtgatcgcgggcctcaacgtcatgcgcataatcaacgagcccaccgccgcggccatcgcctacggcctcgacaagaaggccaccagcaccggggagaagaacgtgctcatcttcgacctcggcggcggcaccttcgacgtgtccatcctcaccatcgaggaaggcatattcgaggtcaagtccaccgccggcgacacccacctgggaggcgaggacttcgacaaccggatggtgaaccacttcgtgcaagagttcaagaggaagaacaagaaggacatcagcggcaacccaagggcgctccggcggctgaggacggcgtgcgagagggccaagaggacgctctcttccaccgcccagaccaccattgagatcgactcgctgtacgaggggatcgacttctacgcgaccatcacccgtgccaggttcgaggagctcaacatggacctcttccgcaagtgcatggagcccgtggagaagtgcctccgggacgccaagatggacaagacccaaatccacgacatcgtgctcgtcggaggctccacccggatccccaaggtgcagcagctcctccaggacttcttcaacgggaaggagctctgcaagagcatcaaccccgacgaggccgtcgcgtacggcgccgccgtgcaggccgccatcctcagcggcgagggcaaccaaaaggtgcaggacctgctcctgctcgacgtgacgccgctctcgctcgggttggagacggccggaggcgtgatgaccaccctgatcccgaggaacaccaccatcccaaccaagaaggagcaggtcttctccacctactcggacaaccagcccggcgtgctgatccaggtgtacgagggcgagaggacgaggaccaaggacaacaacctgctgggcaagttcgagctgtccggcatcccgccggcgcccaggggcgtgccccagatcacggtgaccttcgacatcgacgcgaacggcatcctgaacgtgtcggcagaggacaagacgacgggacagaagaacaagatcaccatcaccaacgacaagggtcggctgagcaaggaggagatcgagcgcatggtgcaggaggcggagaagtacaagtcggaggacgagcaggtgcggcacaaggtggaggcccgcaacgcgctggagaactacgcgtacaacatgcgcaacacgctgcgggacgacaagatcgcgtccaagctccccgccgacgacaagaagaagatcgaggactcgatcgaggacgccatcaagtggctcgacggcaaccagctcgccgaggcggacgagttcgaggacaagatgaaggagctggagagcatctgcaaccccatcatctccaagatgtaccagggcgccggcccgggcggcgcggccggcatggacgaggacatgcccggcggcggcgcgggcaccggcggcgggagcggtgccgggcccaagatcgaagaagtggactaa

SEQ ID NO: 39 - protein, 657 aa, Triticum aestivum
MASKGGNKGEGPAIGIDLGTTYSCVGVWQHDRVEIVANDQGNRTTPSYVAFTDTERLIGDAAKNQVAMNPTNTVFDAKRLIGRRFSDASVQSDMKMWPFKVVPGAGDKPMIVVTYKGEEKTFSAEEISSMVLTKMREIAEAFLSTTINNAVVTVPAYFNDSQRQATKDAGVIAGLNVMRIINEPTAAAIAYGLDKKATSTGEKNVLIFDLGGGTFDVSILTIEEGIFEVKSTAGDTHLGGEDFDNRMVNHFVQEFKRKNKKDISGNPRALRRLRTACERAKRTLSSTAQTTIEIDSLYEGIDFYATITRARFEELNMDLFRKCMEPVEKCLRDAKMDKTQIHDIVLVGGSTRIPKVQQLLQDFFNGKELCKSINPDEAVAYGAAVQAAILSGEGNQKVQDLLLLDVTPLSLGLETAGGVMTTLIPRNTTIPTKKEQVFSTYSDNQPGVLIQVYEGERTRTKDNNLLGKFELSGIPPAPRGVPQITVTFDIDANGILNVSAEDKTTGQKNKITITNDKGRLSKEEIERMVQEAEKYKSEDEQVRHKVEARNALENYAYNMRNTLRDDKIASKLPADDKKKIEDSIEDAIKWLDGNQLAEADEFEDKMKELESICNPIISKMYQGAGPGGAAGMDEDMPGGGAGTGGGSGAGPKIEEVD

SEQ ID NO: 40 - DNA, 1974 bp, Triticum aestivum
  source 1..1974: mol_type = genomic DNA
atggcgagcaagggaggcaacaagggggagggccctgcaatcggcatcgacctcggcaccacatactcctgcgtcggcgtctggcagcatgaccgggtggagatcgtcgccaacgaccagggcaaccgcaccacgccgtcctacgtcgccttcaccgacaccgagcggctcatcggcgacgccgccaagaaccaggtcgccatgaaccccaccaacaccgtttttgatgccaagcgactcatcggacggcgcttctcggacgcatccgtgcagtcggatatgaagatgtggccgttcaaggtggtccccggcgccggcgacaagccgatgatcgtggtcacctacaagggggaggagaagaccttctctgccgaggagatatcctccatggtgctcaccaagatgagggagatcgccgaggccttcctcagcacaaccatcaacaacgccgtcgtcactgtcccggcctacttcaacgactcccagcgccaggccaccaaggacgccggcgtcatcgccggcctcaacgtcatgcgcatcatcaacgagccgaccgcggcggccatcgcttacggcctcgacaaaaaggccaccagcaccggggagaagaacgtgctcatcttcgacctcggcggcggcaccttcgatgtgtccatcctcaccatcgaggaaggcatattcgaggtcaagtccaccgccggggacacccacctggggggcgaggacttcgacaaccggatggtgaaccacttcgtgcaagagttcaagaggaagaacaagaaggacatcagcggcaacccgagggcgctccggcggctgaggacggcgtgcgagagggccaagaggacgctctcttccaccgcccagaccaccattgagattgactcgctctacgaggggattgacttctacgcgaccatcacccgtgccaggttcgaggagctcaacatggacctcttccgcaagtgcatggagcccgtcgagaagtgcctccgggacgccaagatggacaagacccaaatccacgacatcgtgctcgtcggaggctccacccggatccccaaggtgcagcagctcctccaggacttcttcaacgggaaggagctctgcaagagcatcaaccccgacgaggccgtcgcgtacggcgccgccgtgcaggctgccatcctcagcggcgagggcaatcagaaggtgcaggacctgctcctgctcgatgtgacgccgctctcgctcgggttggagacggccggaggcgtgatgactactctgatcccgaggaacaccaccatccccaccaagaaggagcaggtcttctccacctactcggacaaccagcccggcgtgctgatccaggtgtacgagggcgagaggacgaggaccaaggacaacaacctgctgggcaagttcgagctgtccggcatcccgccggcgcccaggggcgtcccccagatcacggtgaccttcgacatcgacgcgaacggcatcctgaacgtgtccgcggaggacaagacgaccgggcagaagaacaagatcaccatcaccaacgacaaggggcggctgagcaaggaggagatcgagcgcatggtgcaggaggcggagaagtacaagtctgaggacgagcaggtgcggcacaaggtggaggcccgcaacgcgctggagaactacgcgtacaacatgcgcaacacggtgcgggacgagaagatcgcgtccaagctccccgccgaagacaagaagaagatcgaggactccatcgaggacgccatcaagtggctcgacggcaaccagctggccgaggccgacgagttcgaggacaagatgaaggagctggagaacatctgcaaccccatcatctccaagatgtaccagggcgccggcccgggcggcgcggccggcatggacgaggacatgcccggcggcggcgcgggcaccggcggtgggagcggtgccgggcccaagatcgaagaagtggactga

SEQ ID NO: 41 - protein, 657 aa, Triticum aestivum
MASKGGNKGEGPAIGIDLGTTYSCVGVWQHDRVEIVANDQGNRTTPSYVAFTDTERLIGDAAKNQVAMNPTNTVFDAKRLIGRRFSDASVQSDMKMWPFKVVPGAGDKPMIVVTYKGEEKTFSAEEISSMVLTKMREIAEAFLSTTINNAVVTVPAYFNDSQRQATKDAGVIAGLNVMRIINEPTAAAIAYGLDKKATSTGEKNVLIFDLGGGTFDVSILTIEEGIFEVKSTAGDTHLGGEDFDNRMVNHFVQEFKRKNKKDISGNPRALRRLRTACERAKRTLSSTAQTTIEIDSLYEGIDFYATITRARFEELNMDLFRKCMEPVEKCLRDAKMDKTQIHDIVLVGGSTRIPKVQQLLQDFFNGKELCKSINPDEAVAYGAAVQAAILSGEGNQKVQDLLLLDVTPLSLGLETAGGVMTTLIPRNTTIPTKKEQVFSTYSDNQPGVLIQVYEGERTRTKDNNLLGKFELSGIPPAPRGVPQITVTFDIDANGILNVSAEDKTTGQKNKITITNDKGRLSKEEIERMVQEAEKYKSEDEQVRHKVEARNALENYAYNMRNTVRDEKIASKLPAEDKKKIEDSIEDAIKWLDGNQLAEADEFEDKMKELENICNPIISKMYQGAGPGGAAGMDEDMPGGGAGTGGGSGAGPKIEEVD

SEQ ID NO: 42 - DNA, 1974 bp, Triticum aestivum
  source 1..1974: mol_type = genomic DNA
atggcgagcaagggaggcaacaagggggagggccctgcgatcggcatcgacctcggcaccacctactcctgcgtcggcgtatggcagcacgaccgggtggagatcgtcgccaacgaccaaggcaaccgcaccacgccgtcctacgtcgccttcaccgacaccgagcggctcatcggcgacgccgccaagaaccaggtcgccatgaaccccaccaacaccgtttttgatgccaagcggctcatcgggcggcgcttctcggacgcatccgtgcagtcggacatgaagatgtggccgttcaaggtggtccccggcgccggcgacaaaccgatgatcgtggtcagctacaagggggaggagaagaccttctccgccgaggagatatcctccatggtgctcaccaagatgagggagatcgccgaggccttcctcagcacgaccatcaacaacgccgtcgtcaccgtcccggcgtacttcaacgactcccagcgccaggccaccaaggacgccggcgtgatcgcgggtctcaacgtcatgcgcataatcaacgagcccaccgcggcggccatcgcttacggcctcgacaagaaggccaccagcaccggggagaagaacgtgctcatcttcgacctcggcggcggcaccttcgatgtgtccatcctcaccatcgaggaaggcatattcgaggtcaagtccaccgccggcgacacccacctgggaggcgaggacttcgacaaccggatggtgaaccacttcgtgcaagagttcaagaggaagaacaagaaggacatcagcggcaacccgagggcgctccggcggctgaggacggcgtgcgagagggcgaagaggaccctctcttccaccgcccagaccaccattgagatcgactcgctctacgaggggatcgacttctacgcgaccatcacccgtgccaggttcgaggagctcaacatggacctcttccgcaagtgcatggagcccgtcgagaagtgcctccgggacgccaagatggacaagacccaaatccacgacatcgtgctcgtcggaggctccacccggatccccaaggtgcagcagctcctccaggacttcttcaacgggaaggagctctgcaagagcatcaaccccgacgaggccgtcgcgtacggcgccgccgtccaggccgccatcctcagcggcgagggcaaccagaaggtgcaggacctgctcctgctcgacgtgacgccgctctcgctcgggttggagacggccggaggcgtgatgacgaccctgatcccgaggaacaccaccatccccaccaagaaggagcaggtcttctcaacctactcggacaaccagcccggcgtgctgatccaggtgtacgagggcgagaggacgaggaccaaggacaacaacctgctgggcaagttcgagctgtccggcatcccgtcggcgcccaggggcgtaccccagatcacggtgaccttcgacatcgacgcgaacggcatcctgaacgtgtcggcggaggacaagacgaccgggcagaagaacaagatcaccatcaccaacgacaaggggcggctgagcaaggaggagatcgagcgcatggtgcaggaggcggagaagtacaagtcggaggacgagcaggtgcggcacaaggtggaggcccgcaacgcgctggagaactacgcgtacaacatgcgcaacacggtgcgggacgagaagatcgcgtccaagctccccgccgacgacaagaagaagatcgaggactccatcgaggacgccatcaagtggctcgacggcaaccagctcgccgaggccgacgagttcgaggacaagatgaaggagctggagagcatctgcaaccccatcatctccaagatgtaccagggtgctggcccgggcggcgcggccggcatggacgaggacatgcccggcggcggcgcgggcaccggcggtgggagcggtgccgggcccaagatcgaagaagtggactga

SEQ ID NO: 43 - protein, 657 aa, Triticum aestivum
MASKGGNKGEGPAIGIDLGTTYSCVGVWQHDRVEIVANDQGNRTTPSYVAFTDTERLIGDAAKNQVAMNPTNTVFDAKRLIGRRFSDASVQSDMKMWPFKVVPGAGDKPMIVVSYKGEEKTFSAEEISSMVLTKMREIAEAFLSTTINNAVVTVPAYFNDSQRQATKDAGVIAGLNVMRIINEPTAAAIAYGLDKKATSTGEKNVLIFDLGGGTFDVSILTIEEGIFEVKSTAGDTHLGGEDFDNRMVNHFVQEFKRKNKKDISGNPRALRRLRTACERAKRTLSSTAQTTIEIDSLYEGIDFYATITRARFEELNMDLFRKCMEPVEKCLRDAKMDKTQIHDIVLVGGSTRIPKVQQLLQDFFNGKELCKSINPDEAVAYGAAVQAAILSGEGNQKVQDLLLLDVTPLSLGLETAGGVMTTLIPRNTTIPTKKEQVFSTYSDNQPGVLIQVYEGERTRTKDNNLLGKFELSGIPSAPRGVPQITVTFDIDANGILNVSAEDKTTGQKNKITITNDKGRLSKEEIERMVQEAEKYKSEDEQVRHKVEARNALENYAYNMRNTVRDEKIASKLPADDKKKIEDSIEDAIKWLDGNQLAEADEFEDKMKELESICNPIISKMYQGAGPGGAAGMDEDMPGGGAGTGGGSGAGPKIEEVD

SEQ ID NO: 44 - DNA, 1259 bp, Zea mays
  source 1..1259: mol_type = genomic DNA
cgatatttgccaaaagtttacggttttgtatttcatcgtgtcgcccaagaaaatcgaaggaacgcgccgccaccggccgcaacaaagcaacgcaggcctccggcctccctgaactgacttcagaagtcaccaacgcacccagcacagacagaagaccctcgccggacgctcgctctcgccggggacaactcaatcacgaggccaggatggcccgctacgatcgcgcgatcaccgtgttctcacccgacggccacctcttccaggtcgagtacgccctcgaggccgtccgcaagggcaacgccgctgtcggcgtccgcggtgtcgacaccgtcgtcctcggcgtcgagaagaagtccacccccaagctccaggactccaggtcggtccgtcggtacctccccctccctcccatccaccagatctcttgaccccgggcgccgcctcacccaaatcctcccccaaaaaatctccgcaggtccgtgcgcaagatcgcgagcctggacacccacatcgcgctggcgtgcgcggggctcaaggccgacgcgcgcgtgctcatcaaccgcgctcgcgtggagtgccagagccaccgcctcacagtcgaggaccccgtcaccgtcgagtacatcacgcgctacatcgccggcctgcagcagaagtacacgcagagcggaggggtgcgccccttcggcctctccacgctcatcgtcggcttcgatccctacacccagaagcccgcgctgtaccagactgacccctcggggaccttctccgcttggaaggccaacgcaaccggccgcaactccaactccatgcgcgagtttctcgagaagaattacaaggagacgtccggtaaggagaccatcaagctcgccatcagagcactccttgaggtatacactcaagcacttaattagcactagtggatccaaacaggcccttttatgtgattattcagagagacatagtacttgaagggtgtcaattgggagcaattagtatagaatgagaaagatcaactccttatttataaaggcgacaatttgtcactgaagtgaaccaaatgatgtggcagtgctggacttagaactaagcttgcattgctcaactcaatgtaccactggagtgttgaaatctatactttgtgtgtgctgtggcagccaagtgtttcatgtgtcatggttgattcattatgaatagtggtagcatcaactatagatcccagtttaaacgatagttgcagatttcaatgaaatttttat

SEQ ID NO: 45 - DNA, 750 bp, Zea mays
  source 1..750: mol_type = genomic DNA
atggcccgctacgatcgcgcgatcaccgtgttctcacccgacggccacctcttccaggtcgagtacgccctcgaggccgtccgcaagggcaacgccgctgtcggcgtccgcggtgtcgacaccgtcgtcctcggcgtcgagaagaagtccacccccaagctccaggactccaggtccgtgcgcaagatcgcgagcctggacacccacatcgcgctggcgtgcgcggggctcaaggccgacgcgcgcgtgctcatcaaccgcgctcgcgtggagtgccagagccaccgcctcacagtcgaggaccccgtcaccgtcgagtacatcacgcgctacatcgccggcctgcagcagaagtacacgcagagcggaggggtgcgccccttcggcctctccacgctcatcgtcggcttcgatccctacacccagaagcccgcgctgtaccagactgacccctcggggaccttctccgcttggaaggccaacgcaaccggccgcaactccaactccatgcgcgagtttctcgagaagaattacaaggagacgtccggtaaggagaccatcaagctcgccatcagagcactccttgaggttgttgagagtggtggcaagaacatagagattgcagtgatgacacacgaggacggccttcatgaactcgaagaggctgagattgatgagtatgttgccgaaattgaagcagagaaagccgctgccgaggctgcaaagaagggtgcaccaaaggggaactga

SEQ ID NO: 46 - protein, 249 aa, Zea mays
MARYDRAITVFSPDGHLFQVEYALEAVRKGNAAVGVRGVDTVVLGVEKKSTPKLQDSRSVRKIASLDTHIALACAGLKADARVLINRARVECQSHRLTVEDPVTVEYITRYIAGLQQKYTQSGGVRPFGLSTLIVGFDPYTQKPALYQTDPSGTFSAWKANATGRNSNSMREFLEKNYKETSGKETIKLAIRALLEVVESGGKNIEIAVMTHEDGLHELEEAEIDEYVAEIEAEKAAAEAAKKGAPKGN

SEQ ID NO: 47 - DNA, 750 bp, Triticum aestivum
  source 1..750: mol_type = genomic DNA
atggcccgctacgaccgagcgatcaccgtcttctcccccgacggccacctcttccaggttgagtacgccctcgaggccgtccgcaagggcaacgccgccgtcggcgtccgaggcgtcgacaccgtcgtcctcggtgtcgagaagaagtccacccccaagctccaggactccaggtccgtgcgcaagatcgcgagcctggacacccacatcgcgctggcctgcgcgggtctgaaggcggacgcgcgcgtgctcatcaaccgggcgcgcgtcgagtgccagagccaccgcctcaccgtcgaggaccccgtcactgtcgagtacatcacgcgctacatcgccggcctgcagcagaagtacacgcagagcggaggggtgcgcccgttcgggctctccacgctcatagttggcttcgacccctataccgagaagcccgcgctgtaccagaccgacccctccggcaccttctccgcctggaaggccaacgccaccggccgcaactccaactccatgcgcgaattccttgagaagaattacaaggagacgtccggcaaggagaccatcaagcttaccatccgagccctccttgaggttgtcgagagtggtggcaagaacattgagattgctgtgatgacgcacaaagatggtcttcgccagcttgaggaagaggagatcgatgagtatgttgctgagattgaagcagagaaggccgctgctgaggctgcaaagaagggtggcccgaaagacacataa

SEQ ID NO: 48 - protein, 249 aa, Triticum aestivum
MARYDRAITVFSPDGHLFQVEYALEAVRKGNAAVGVRGVDTVVLGVEKKSTPKLQDSRSVRKIASLDTHIALACAGLKADARVLINRARVECQSHRLTVEDPVTVEYITRYIAGLQQKYTQSGGVRPFGLSTLIVGFDPYTEKPALYQTDPSGTFSAWKANATGRNSNSMREFLEKNYKETSGKETIKLTIRALLEVVESGGKNIEIAVMTHKDGLRQLEEEEIDEYVAEIEAEKAAAEAAKKGGPKDT

SEQ ID NO: 49 - DNA, 750 bp, Triticum aestivum
  source 1..750: mol_type = genomic DNA
atggcccgctacgaccgagcgatcaccgtcttctcccccgacggccacctcttccaggttgagtacgccctcgaggccgtccgcaagggcaacgccgccgtcggcgtccgaggcgtcgacaccgtcgtcctcggtgtcgagaagaagtccacccccaagctccaagactccaggtccgtgcgcaagatcgcgagcctggacacccacatcgcgttggcctgcgcgggtctgaaggcggacgcgcgcgtgctcatcaaccgggcgcgcgtcgagtgccagagccaccgcctcaccgtcgaggaccccgtcactgtcgagtacatcacgcgctacatcgccggcctgcagcagaagtacacgcagagcggaggggtgcgcccgttcgggctctccacgctcatagttggcttcgacccctatactgagaagcctgcgctgtaccagaccgacccctccggcaccttctccgcctggaaggccaacgccaccggccgcaactccaactccatgcgcgagttccttgagaagaattacaaggagacgtccggcaaggagaccatcaagcttaccatccgagccctccttgaggttgtcgagagtggtggcaagaacattgagattgctgtgatgacgcacaaagatggtcttcgccagcttgaggaagaggagatcgatgagtatgttgctgagattgaagcagagaaggctgctgctgaggctgcgaagaagggtggcccgaaagacacataa

SEQ ID NO: 50 - protein, 249 aa, Triticum aestivum; identical to SEQ ID NO: 48

SEQ ID NO: 51 - DNA, 750 bp, Triticum aestivum
  source 1..750: mol_type = genomic DNA
atggcccgctacgaccgagcgatcaccgtcttctcccccgacggccacctcttccaggttgagtacgccctcgaggccgtccgcaagggcaacgccgccgtcggcgtccgaggcgtcgacaccgtcgtcctcggtgtcgagaagaagtccacccccaagctccaggactccaggtccgtgcgcaagatcgcgagcctggacacccacatcgcgctggcctgcgcgggtctcaaggcggacgcgcgcgtgctgatcaaccgggcgcgcgtcgagtgccagagccaccgcctcaccgtcgaggaccccgtcactgtcgagtacatcacgcgctacatcgccggcctgcagcagaagtacacgcagagcggaggggtgcgcccgttcgggctctccacgcttatagttggcttcgacccctatactgagaagccggcgctgtaccagaccgacccctccggcaccttctccgcctggaaggccaacgccaccggccgcaactccaactccatgcgcgagttccttgagaagaattacaaggagacgtccggcaaggagaccatcaagcttaccatccgagccctccttgaggttgtcgagagtggtggcaagaacattgagattgctgtgatgacgcacaaagatggtcttcgccagcttgaggaagaggagatcgatgagtatgttgctgagattgaagcagagaaggccgctgctgaggctgcgaagaagggtggcccgaaagacacataa

SEQ ID NO: 52 - protein, 249 aa, Triticum aestivum; identical to SEQ ID NO: 48

(19) United States
(12) Patent Application Publication
(10) Pub. No.: US 2026/0327646 A1
(43) Pub. Date: Oct. 22, 2026

(54) STERILE GENES AND RELATED CONSTRUCTS AND APPLICATIONS THEREOF

(71) Applicants: PIONEER OVERSEAS CORPORATION, JOHNSTON, IA (US); SINOBIOWAY BIO-AGRICULTURE GROUP CO. LTD., BEIJING (CN)

(72) Inventors: MARC C ALBERTSEN, GRIMES, IA (US); HUANHUAN DING, BEIJING (CN); TIM FOX, DES MOINES, IA (US); GUIHUA LU, SAN DIEGO, CA (US); GUANFAN MAO, BEIJING (CN); GUOKUI WANG, BEIJING (CN); WEI WANG, Beijing (CN); YUZHEN ZHENG, ZHENGZHOU (CN)

(73) Assignees: PIONEER OVERSEAS CORPORATION, JOHNSTON, IA (US); SINOBIOWAY BIO-AGRICULTURE GROUP CO. LTD., BEIJING (CN)

(21) Appl. No.: 19/563,085

(22) Filed: Mar. 11, 2026

Related U.S. Application Data
(62) Division of application No. 17/597,380, filed on Jul. 5, 2019, now Pat. No. 12,595,489.

(51) Int. Cl. C12N 15/82; C07K 14/415

(52) U.S. Cl. CPC C12N 15/829; C07K 14/415

(57) ABSTRACT

Isolated polynucleotides and polypeptides, and recombinant DNA constructs useful for regulating plant sterility are provided, and methods utilizing these recombinant DNA constructs are also provided. Compositions comprising these recombinant DNA constructs or a modified endogenous sterile gene are further provided.

### FIELD

This disclosure relates to the field of plant sexual reproduction, genetics and breeding, and in particular relates to recombinant DNA constructs useful for regulating sterility of plants, and methods for producing sterile plant.

### BACKGROUND

Hybrid rice has greatly contribution to the global increase of rice productivity. A cytoplasmic male sterile (CMS) lines-based three-line system and a photoperiod/thermo-sensitive genic male sterile (PTGMS) line-based two lines system are used in commercial hybrid rice production (Chang et al., Proceedings of the National Academy of Sciences, 113 (49): 14145-14150 (2016)).

Plant male reproductive development involves a series of events, from stamen meristem specification to pollen grain formation and pollination. Cross-pollination of the fertile transgenic plants to the non-transgenic male sterile plants propagated the male sterile seeds of high purity. Pollen fertility is regulated by day length. For example, rice can be completely sterile when grown under long-day conditions, whereas pollen fertility varies when it is grown under short-day conditions.

The development of female organ mainly refers to the process of ovule development and embryo sac formation. If the female organ development is absent or stagnant, the plant will show female sterility. Ling etc. (1991) divided the female sterility into three categories: 1) defects in pistil development, no style and stigma, and only have a dried ovary; 2) the female organs do not differentiate or staminate; 3) the pistil looks normal, but abnormal in the ovary development. Female sterility is often accompanied by male sterility.

Studies of two distantly related dicotyledons, Arabidopsis thaliana and Antirrhinum majus, has led to the identification of three classes of homeotic genes, acting alone or in combination to determine floral organ identity (Bowman, et al., Development, 112:1 (1991); Carpenter and Coen, Genes Devl., 4:1483 (1990); Schwarz-Sommer, et al., Science, 250:931 (1990)). Several of these genes are transcription factors whose conserved DNA-binding domain has been designated as MADS box (Schwarz-Sommer, et al., supra).

Earlier acting genes that control the identity of flower meristem have also been characterized. Flower meristems are derived from inflorescence meristem in both Arabidopsis and Antirrhinum. Two factors that control the development of meristematic cells into flowers are known. In Arabidopsis, the factors are the products of the LEAFY gene (Weige, et al. Cell 69:843 (1992)) and the APETALA1 gene (Mandel, et al., Nature 360:273 (1992)). When either of these genes is inactivated by mutation, structures combining the properties of flowers and inflorescence develop (Weigel, et al., supra; Irish and Sussex, Plant Cell, 2:741 (1990)). In Antirrhinum, the homologue of the Arabidopsis LEAFY gene is FLORICAULA (Coen, et al., Cell, 63:1311 (1990)) and that of the APETALA1 gene is SQUAMOSA (Huijser, et al., EMBO J., 11:1239 (1992)). The latter pair contains MADS box domains.

Number of male sterile genes have been reported. However, not many reports on the female sterile genes. Recently, great progress has been made in utilization of male sterile genes in developing hybrid seed production technology (Wu et al. Plant Biotechno. J. doi: 10.1111/pbi.12477 (2015)).

Accordingly, there is a need to develop new compositions and methods for altering the sterile characteristics of the target plant. This disclosure provides such compositions and methods.

### SUMMARY

In one aspect, the present disclosure includes an isolated polynucleotide regulating plant sterile trait, comprising: (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52; or (c) the full complement of the nucleotide sequence of (a) or (b), wherein increasing expression of the polynucleotide makes the plants sterile.

In certain embodiments, the polynucleotide comprises the nucleotide sequence of SEQ ID NO: 1, SEQ ID NO: 2, SEQ ID NO: 4, SEQ ID NO: 5, SEQ ID NO: 7, SEQ ID NO: 8, SEQ ID NO: 26, SEQ ID NO: 27, SEQ ID NO: 29, SEQ ID NO: 31, SEQ ID NO: 33, SEQ ID NO: 35, SEQ ID NO: 36, SEQ ID NO: 38, SEQ ID NO: 40, SEQ ID NO: 42, SEQ ID NO: 44, SEQ ID NO: 45, SEQ ID NO: 47, SEQ ID NO: 49, or SEQ ID NO: 51.

In certain embodiments, the polynucleotide encodes a polypeptide comprising the amino acid sequence of SEQ ID NO: 3, SEQ ID NO: 6, SEQ ID NO: 9, SEQ ID NO: 28, SEQ ID NO: 30, SEQ ID NO: 32, SEQ ID NO: 34, SEQ ID NO: 37, SEQ ID NO: 39, SEQ ID NO: 41, SEQ ID NO: 43, SEQ ID NO: 46, SEQ ID NO: 48, SEQ ID NO: 50, or SEQ ID NO: 52.

In another aspect, the present disclosure provides the use of the isolated sterile trait-regulating polynucleotide in a plant to regulate the sterile trait, wherein the isolated polynucleotide comprises (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52; or (c) the full complement of the nucleotide sequence of (a) or (b). Certain embodiments provide for the use of the isolated polynucleotide in a plant to make plant sterile by increasing expression of the polynucleotide.

In another aspect, the present disclosure includes a recombinant DNA construct comprising the isolated sterile trait-regulating polynucleotide operably linked to at least one regulatory element, wherein the polynucleotide comprises (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52; or (c) the full complement of the nucleotide sequence of (a) or (b). In certain embodiments, the at least one regulatory element is a promoter functional in a plant. In certain embodiments the at least one regulatory element is heterologous to the polynucleotide.

In another aspect, the present disclosure includes a modified plant, plant cell or seed with increased expression of a polynucleotide encoding a sterility-regulating polypeptide ZOS3-17, DnaK, or PPT1-1, wherein the plant exhibits sterile trait when compared to a control plant planted under the same conditions.

In certain embodiments, the plant, plant cell, or seed is modified to have increased expression of the sterile trait-regulating gene, wherein the plant or plant produced from said plant cell or seed has a sterile trait when compared to a control plant not having said increased expression. In certain embodiments, the modified plant, plant cell, or seed comprises a recombinant DNA construct comprising a sterile trait polynucleotide operably linked to at least one regulatory element, wherein the polynucleotide comprises (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52; or (c) the full complement of the nucleotide sequence of (a) or (b); thereby increasing expression of the polynucleotide in the modified plant, plant cell, or seed. In certain embodiments, the plant comprises a modified regulatory element, wherein the modified regulatory element increases the expression of an endogenous polynucleotide comprising (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52; or (c) the full complement of the nucleotide sequence of (a) or (b).

In certain embodiments, the plant for the use in the compositions and methods provided herein is selected from the group consisting of rice, maize, soybean, sunflower, sorghum, canola, wheat, alfalfa, cotton, barley, millet, sugar cane and switchgrass.

In another aspect, a rice plant is provided, wherein the rice plant comprises a modified genomic locus, wherein expression of an endogenous polynucleotide encoding the polypeptide having an amino acid sequence of at least 90% sequence identity compared to SEQ ID NO: 3, 6 or 9 is increased.

In another aspect, a maize plant is provided, wherein the maize plant comprises a modified genomic locus, wherein expression of an endogenous polynucleotide encoding the polypeptide having an amino acid sequence of at least 90% sequence identity compared to SEQ ID NO: 28, 37, or 46 is increased.

In another aspect, a wheat plant is provided, wherein the wheat plant comprises a modified genomic locus, wherein expression of an endogenous polynucleotide encoding the polypeptide having an amino acid sequence of at least 90% sequence identity compared to SEQ ID NO: 30, 32, 34, 39, 41, 43, 48, 50 or 52 is increased.

In another aspect, methods are provided for regulating plant sterile trait, comprising increasing the expression or function of a polynucleotide encoding a ZOS3-17, Dnak, or PPT1-1 polypeptide in a plant (e.g., rice, maize, or wheat) to make the plant sterile, wherein the polynucleotide comprises: (a) a polynucleotide with a nucleotide sequence of at least 85% identity to SEQ ID NO: 1, 2, 4, 5, 7, 8, 26, 27, 29, 31, 33, 35, 36, 38, 40, 42, 44, 45, 47, 49 or 51; and (b) a polynucleotide encoding a polypeptide with an amino acid sequence of at least 90% identity to SEQ ID NO: 3, 6, 9, 28, 30, 32, 34, 37, 39, 41, 43, 46, 48, 50 or 52.

In another aspect, a method of identifying one or more alleles associated with sterile trait in a population of plants (e.g., rice, maize, wheat) is provided, wherein the method comprises the steps of: (a) detecting in a population of plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO: 3, 6, 9, 28, 30, 32, 34, 37, 39, 41, 43, 46, 48, 50 or 52 or a sequence that is 90% identical to SEQ ID NO: 3, 6, 9, 28, 30, 32, 34, 37, 39, 41, 43, 46, 48, 50 or 52, wherein the one or more polymorphisms in the genomic region encoding the polypeptide or in the regulatory region controlling expression of the polypeptide is associated with sterile trait; and (b) identifying one or more alleles at the one or more polymorphisms that are associated with sterile trait. Wherein the one or more alleles associated with sterile trait is used for marker assisted selection of a plant with sterile trait.

### BRIEF DESCRIPTION OF THE DRAWINGS AND SEQUENCE LISTINGS

The disclosure can be more fully understood from the following detailed description and the accompanying Drawings and Sequence Listing which form a part of this application.

The official copy of the sequence listing is submitted electronically via EFS-Web as an XML formatted sequence listing with a file named 117597-US-DIV-1.XML created on Mar. 9, 2026, and having a size of 116,000 bytes and is filed concurrently with the specification. The sequence listing comprised in this XML formatted document is part of the specification and is herein incorporated by reference in its entirety.

FIG. 1 shows the relative expression levels of OsZOS3-17 gene in leaves of different transgenic rice lines (DP0640) by real-time PCR analyses. The base expression level in ZH11-TC is set at 1.00, the numbers on the top of the columns are fold-change compared to ZH11-TC rice.

FIG. 2 shows the relative expression levels of OsDnak gene in leaves of different transgenic rice lines (DP1673) by real-time PCR analyses. The base expression level in ZH11-TC is set at 1.00, the numbers on the top of the columns are fold-change compared to ZH11-TC rice.

FIG. 3 shows the relative expression levels of OsPPT1-1 gene in leaves of different transgenic rice lines (DP1675) by real-time PCR analyses. The base expression level in ZH11-TC is set at 1.00, the numbers on the top of the columns are fold-change compared to ZH11-TC rice.

FIG. 4 shows the pollen staining results of DP0640 transgenic rice plant. The pollens from DP0640 plants were fertile as showed by the I₂K-statining.

FIG. 5 shows the seed morphology of DP0640 transgenic rice plant.

FIG. 6 shows the PCR amplification result of F1 hybrid plants of DP0640 transgenic cross-out rice plants.

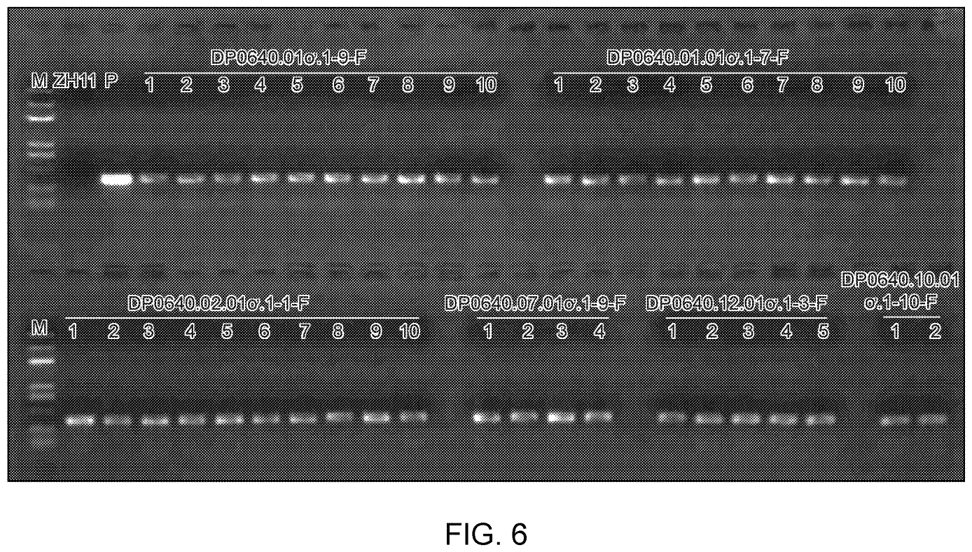

FIG. 7 shows the pollen staining results of DP1673 transgenic rice plant. The pollens from DP1673 plants were fertile as showed by the I₂K-statining.

FIG. 8 shows the pistil development of DP1673 plants. DP1673 plants apparently grew normally before pollination, however, their ovary and stigmas withered and died after pollination.

FIG. 9 shows the pollen staining results of DP1675 transgenic rice plant. The pollens from DP1675 plants were fertile as showed by the I₂K-statining.

FIG. 10 shows the PCR amplification result of F1 hybrid plants of DP1675 transgenic cross-out rice plants.

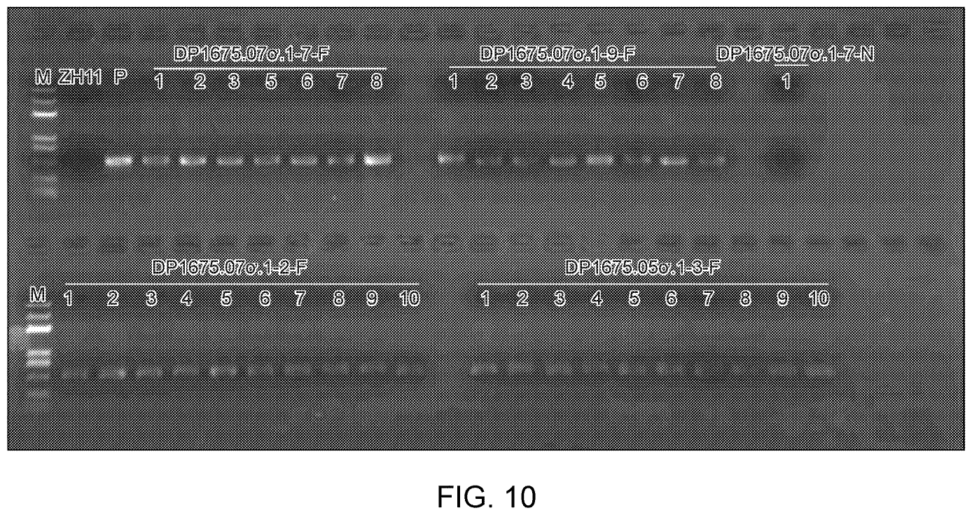

TABLE 1
SEQ ID NOs for nucleotide and amino acid sequences
provided in the sequence listing

| | | | SEQ ID |
|---|---|---|---|
| | | SEQ ID | NO: |
| | | NO: | (Amino |
| Source species | Clone Description | (Nucleotide) | Acid) |
| Oryza sativa | OsZOS3-17 cDNA | 1 | |
| Oryza sativa | OsZOS3-17 CDS | 2 | |
| Oryza sativa | OsZOS3-17 PRT | | 3 |
| Oryza sativa | OsDnaK cDNA | 4 | |
| Oryza sativa | OsDnaK CDS | 5 | |
| Oryza sativa | OsDnaK PRT | | 6 |
| Oryza sativa | OsPPT1-1 gDNA | 7 | |
| Oryza sativa | OsPPT1-1 CDS | 8 | |
| Oryza sativa | OsPPT1-1 PRT | | 9 |
| Artificial | Primers | 10-25 | n/a |
| Zea mays | ZOS3-17 gDNA | 26 | |
| Zea mays | ZOS3-17 CDS | 27 | |
| Zea mays | ZOS3-17 PRT | | 28 |
| Triticum aestivum | ZOS3-17 from A genome | 29 | |
| | CDS | | |
| Triticum aestivum | ZOS3-17 from A genome | | 30 |
| | PRT | | |
| Triticum aestivum | ZOS3-17 from B genome | 31 | |
| | CDS | | |
| Triticum aestivum | ZOS3-17 from B genome | | 32 |
| | PRT | | |
| Triticum aestivum | ZOS3-17 from D genome | 33 | |
| | CDS | | |
| Triticum aestivum | ZOS3-17 from D genome | | 34 |
| | PRT | | |
| Zea mays | DnaK gDNA | 35 | |
| Zea mays | DnaK CDS | 36 | |
| Zea mays | DnaK PRT | | 37 |
| Triticum aestivum | DnaK from A genome | 38 | |
| | CDS | | |
| Triticum aestivum | DnaK from A genome | | 39 |
| | PRT | | |
| Triticum aestivum | DnaK from B genome | 40 | |
| | CDS | | |
| Triticum aestivum | DnaK from B genome | | 41 |
| | PRT | | |
| Triticum aestivum | DnaK from D genome | 42 | |
| | CDS | | |
| Triticum aestivum | DnaK from D genome | | 43 |
| | PRT | | |
| Zea mays | PPT1-1 gDNA | 44 | |
| Zea mays | PPT1-1 CDS | 45 | |
| Zea mays | PPT1-1 PRT | | 46 |
| Triticum aestivum | PPT1-1 from A genome | 47 | |
| | CDS | | |
| Triticum aestivum | PPT1-1 from A genome | | 48 |
| | PRT | | |
| Triticum aestivum | PPT1-1 from B genome | 49 | |
| | CDS | | |
| Triticum aestivum | PPT1-1 from B genome | | 50 |
| | PRT | | |
| Triticum aestivum | PPT1-1 from D genome | 51 | |
| | CDS | | |
| Triticum aestivum | PPT1-1 from D genome | | 52 |
| | PRT | | |

The Drawing Descriptions and Sequence Listing attached hereto comply with the rules governing nucleotide and/or amino acid sequence disclosures in patent applications as set forth in 37 C.F.R. § 1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2): 345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

“OsZOS3-17” refers to a rice polypeptide that confers a sterile phenotype when expression is altered. The OsZOS3-17 polypeptide (SEQ ID NO: 3) is encoded by the coding sequence (CDS) (SEQ ID NO: 2) or nucleotide sequence (SEQ ID NO: 1) at rice gene locus LOC_Os03g50850.1 which is annotated as “ZOS3-17-C2H2 zinc finger protein, expressed” in TIGR. “ZOS3-17 polypeptide” refers herein to the OsZOS3-17 polypeptide and its paralogs or homologs from other organisms, such as maize (e.g., SEQ ID NO: 28 encoded by SEQ ID NO: 27 or SEQ ID NO: 26) or wheat (e.g., SEQ ID NO: 30 encoded by SEQ ID NO: 29; SEQ ID NO: 32 encoded by SEQ ID NO: 31; SEQ ID NO: 34 encoded by SEQ ID NO: 33).

“OsDnaK” refers to a rice polypeptide that confers a sterile phenotype when expression is altered. The OsDnak polypeptide (SEQ ID NO: 6) is encoded by the coding sequence (CDS) (SEQ ID NO: 5) or nucleotide sequence (SEQ ID NO: 4) at rice gene locus LOC_Os03g16920.1 which is annotated as “Dnak family protein, putative, expressed” in TIGR. “Dnak polypeptide” refers herein to the OsDnak polypeptide and its paralogs or homologs from other organisms, such as maize (e.g., SEQ ID NO: 37 encoded by SEQ ID NO: 36 or SEQ ID NO: 35) or wheat (e.g., SEQ ID NO: 39 encoded by SEQ ID NO: 38; SEQ ID NO: 41 encoded by SEQ ID NO: 40; SEQ ID NO: 43 encoded by SEQ ID NO: 42).

“OsPPT1-1” refers to a rice polypeptide that confers a sterile phenotype when expression is altered. The OsPPT1-1 polypeptide (SEQ ID NO: 9) is encoded by the coding sequence (CDS) (SEQ ID NO: 8) or nucleotide sequence (SEQ ID NO: 7) at rice gene locus LOC_Os08g43540.1 which is annotated as “peptidase, T1 family, putative, expressed” in TIGR. “PPT1-1 polypeptide” refers herein to the OsPPT1-1 polypeptide and its paralogs or homologs from other organisms, such as maize (e.g., SEQ ID NO: 46 encoded by SEQ ID NO: 45 or SEQ ID NO: 44) or wheat (e.g., SEQ ID NO: 48 encoded by SEQ ID NO: 47; SEQ ID NO: 50 encoded by SEQ ID NO: 49; SEQ ID NO: 52 encoded by SEQ ID NO: 51).

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some embodiments, the wheat plant is suitable for commercial production of grain, such as commercial varieties of hexaploid wheat or durum wheat, having suitable agronomic characteristics which are known to those skilled in the art.

The terms “sterile” and “sterility” refers to the plants cannot produce seeds. Include but not limited to, the pollen fertility rate or seed setting rate less than 20%, the female organ development is absent or stagnant. The said female organ include but not limited to ovule, pistil, style, stigma etc.

The terms “semi-sterile” and “semi-sterility” refers to the plants cannot produce seeds and/or pollen properly. Include but not limited to, the pollen fertility rate or seed setting rate less than 80%, more than 20%.

The terms “fertile” and “fertility” refers to the plants can produce seeds normally. Include but not limited to, the pollen fertility rate or seed setting rate more than 80%, and the said seeds can germinate and grow normally.

The terms “male sterile” refers to the plants cannot produce pollen or cannot produce enough pollen, which is influenced by the plant reproductive development, such as from stamen meristem specification to pollen grain formation and pollination.

The terms “female sterile” refers to the plants cannot produce seeds because of the abnormal development of female organ. The said abnormal development of female organ, includes but not limited to, the process of ovule development and embryo sac formation.

The terms “full complement” and “full-length complement” are used interchangeably herein and refer to a complement of a given nucleotide sequence, wherein the complement and the nucleotide sequence consist of the same number of nucleotides and are 100% complementary.

The term “trait” refers to a physiological, morphological, biochemical, or physical characteristic of a plant or particular plant material or cell.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Transgenic” refers to any cell, cell line, callus, tissue, plant part or plant, the genome of which has been altered by the presence of a recombinant DNA construct.

A “control” or “control plant” or “control plant cell” provides a reference point for measuring changes in phenotype of a subject plant or plant cell which was genetically altered by, such as transformation, and has been affected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to a condition or stimulus that would induce expression of the gene of interest; or (e) the subject plant or plant cell itself, under conditions in which the gene of interest is not expressed.

In this disclosure, ZH11-WT, ZH11-TC, WT and empty vector plants may be designated as control plants. ZH11-WT represents wild type Zhonghua 11, ZH11-TC represents rice plants generated from tissue cultured Zhonghua 11, WT represents the wild type plants, such as Zhonghua 11, Daohuaxiang 2, and empty vector represents plants transformed with empty vector DP0158.

“Genome” as it applies to plant cells encompasses not only chromosomal DNA found within the nucleus, but organelle DNA found within subcellular components (e.g., mitochondrial, plastid) of the cell.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

The term “gene” refers to a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. A mutated or modified plant is a plant comprising a mutated gene.

A “targeted mutation” is a mutation in a native gene that was made by altering a target sequence within the native gene using a method involving a double-strand-break-inducing agent that is capable of inducing a double-strand break in the DNA of the target sequence as disclosed herein of known in the art.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Heterologous” with respect to sequence means a sequence that originates from a foreign species, or, if from the same species, is substantially modified from its native form in composition and/or genomic position by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified but does not encompass the alteration of the cell or vector by naturally occurring events (e.g., spontaneous mutation, natural transformation/transduction/transposition) such as those occurring without deliberate human intervention.

“Non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments, the non-genomic nucleic acid molecule is a cDNA. In some embodiments, the non-genomic nucleic acid molecule is a synthetic nucleic acid sequence.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source but arranged in a manner different than that normally found in nature.

“Regulatory sequences” and “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably and refer to a promoter that is expressed predominantly but not necessarily exclusively in one tissue or organ, but that may also be expressed in one specific cell.

“Developmentally regulated promoter” refers to a promoter whose activity is determined by developmental events.

“Genetic modification” refers to a change or alteration in the genomic nucleic acid sequence of a plant introduced by deliberate human activity.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Transient transformation” refers to the introduction of a nucleic acid fragment into the nucleus, or DNA-containing organelle, of a host organism resulting in gene expression without genetically stable inheritance.

“Stable transformation” refers to the introduction of a nucleic acid fragment into a genome of a host organism resulting in genetically stable inheritance.

A “nuclear localization signal” is a signal peptide which directs the protein to the nucleus (Raikhel. (1992) Plant Phys. 100:1627-1632).

A “suppression DNA construct” is a recombinant DNA construct which when transformed or stably integrated into the genome of the plant, results in “silencing” of a target gene in the plant. The target gene may be endogenous or transgenic to the plant. “Silencing”, as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, includes lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product.

“Cosuppression” refers to the production of sense RNA transcripts capable of suppressing the expression of the target gene or gene product. “Sense” RNA refers to RNA transcript that includes the mRNA and can be translated into protein within a cell or in vitro.

Another variation describes the use of plant viral sequences to direct the suppression of proximal mRNA encoding sequences (PCT Publication No. WO 98/36083 published on Aug. 20, 1998).

RNA interference (RNAi) refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants.

MicroRNAs (miRNAs) are designed that regulate target genes (e.g., the polynucleotide sequences disclosed herein) by binding to complementary sequences located in the transcripts produced by these genes for example by translational inhibition and RNA cleavage.

“CRISPR-associated genes” refers to nucleic acid sequences that encode polypeptide components of clustered regularly interspersed short palindromic repeats (CRISPR)-associated systems (Cas), and the genes are generally coupled, associated or close to or in the vicinity of flanking CRISPR loci. The terms “Cas gene”, “CRISPR-associated gene” are used interchangeably herein. Examples include, but are not limited to, Cas3 and Cas9, which encode endonucleases from the CRISPR type I and type II systems, respectively.

“Cas endonuclease” refers to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell.

“Guide RNA (gRNA)” refers to a crRNA (CRISPR RNA):tracrRNA fused hybrid RNA molecule encoded by a customizable DNA element that, generally, comprises a copy of a spacer sequence which is complementary to the protospacer sequence of the genomic target site, and a binding domain for an associated-Cas endonuclease of the CRISPR complex.

“Guide polynucleotide” refers to a polynucleotide sequence that can form a complex with a Cas endonuclease and enables the Cas endonuclease to recognize and optionally cleave a DNA target site. The guide polynucleotide can be comprised of a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

The term “guide polynucleotide/Cas endonuclease system” refers to a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

“Genomic target site” refers to a protospacer and a protospacer adjacent motif (PAM) located in a host genome selected for targeted mutation and/or double-strand break.

“Protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sgRNA.

“Protospacer adjacent motif (PAM)” includes a 3 to 8 bp sequence immediately adjacent to the protospacer sequence in the genomic target site.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 bp by depending on the CRISPR locus (WO2007/025097 published Mar. 1, 2007).

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (patent application WO-PCT PCT/US12/30061 filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, Foki. The unique, modular TAL effector DNA binding domain allows for the design of proteins with potentially any given DNA recognition specificity (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type 1 is endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Turning now to the embodiments:

Embodiments include isolated polynucleotides and polypeptides, recombinant DNA constructs (including suppression constructs) useful for regulating plant sterile trait, compositions (such as plants or seeds) comprising these recombinant DNA constructs, and methods utilizing these recombinant DNA constructs, compositions comprising sterile-regulating gene and its promoter.

### Isolated Polynucleotides and Polypeptides

The present disclosure includes the following isolated polynucleotides and polypeptides:

In some embodiments, isolated polynucleotides are provided comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 3, 6, 9, 28, 30, 32, 34, 37, 39, 41, 43, 46, 48, 50, or 52; or (ii) a full complement of the nucleic acid sequence of (i), wherein the full complement and the nucleic acid sequence of (i) consist of the same number of nucleotides and are 100% complementary. In certain embodiments, increasing expression of this polynucleotide makes the plant sterile. Any of the foregoing isolated polynucleotides may be utilized in any recombinant DNA constructs of the present disclosure.

In some embodiments, isolated polypeptides are provided having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 3, 6, 9, 28, 30, 32, 34, 37, 39, 41, 43, 46, 48, 50, or 52.

In some embodiments, isolated polynucleotides are provided comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 1, 2, 4, 5, 7, 8, 26, 27, 29, 31, 33, 35, 36, 38, 40, 42, 44, 45, 47, 49 or 51; or (ii) a full complement of the nucleic acid sequence of (i). Any of the foregoing isolated polynucleotides may be utilized in any recombinant DNA constructs of the present disclosure. In certain embodiments, increasing expression of this polynucleotide makes plant sterile.

### Recombinant DNA Constructs

In one aspect, the present disclosure includes recombinant DNA constructs.

In one embodiment, the recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 3, 6, 9, 28, 30, 32, 34, 37, 39, 41, 43, 46, 48, 50, or 52; or (ii) a full complement of the nucleic acid sequence of (i).

In another embodiment, the recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity as compared to SEQ ID NO: 1, 2, 4, 5, 7, 8, 26, 27, 29, 31, 33, 35, 36, 38, 40, 42, 44, 45, 47, 49 or 51; or (ii) a full complement of the nucleic acid sequence of (i).

In another embodiment, the recombinant DNA construct comprises a polynucleotide operably linked to at least one regulatory sequence (e.g., a promoter functional in a plant), wherein said polynucleotide encodes a OsZOS3-17, Dnak or a PPT1-1 protein. These polypeptides regulate sterile trait, and may be from, for example, Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Zea mays, Glycine max, Glycine tabacina, Glycine soja or Glycine tomentella.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### Regulatory Elements:

A recombinant DNA construct (including a suppression DNA construct) of the present disclosure may comprise at least one regulatory element.

A regulatory element may be a promoter, enhancer, 5′UTR, or 3′UTR.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may (or may not) have pleiotropic effects, although candidate gene efficacy may be estimated when driven by a constitutive promoter. Use of tissue-specific and/or stress-specific promoters may eliminate undesirable effects, but retain the ability to regulate plant flowering time. This type of effect has been observed in Arabidopsis for drought and cold tolerance (Kasuga et al., Nature Biotechnol. 17:287-91 (1999)).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter which causes the desired temporal and spatial expression may be used in the methods of the present disclosure.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development, the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter ltp2 (U.S. Pat. No. 5,525,716); maize Zm40 promoter (U.S. Pat. No. 6,403,862); maize nuclc (U.S. Pat. No. 6,407,315); maize ckx1-2 promoter (U.S. Pat. No. 6,921,815 and US Patent Application Publication Number 2006/0037103); maize lec1 promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eep1 (U.S. Patent Application Publication Number 20070169226); and maize promoter ADF4 (U.S. Patent Application No. 60/963,878, filed 7 Aug. 2007). Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (GenBank Accession No. EF030817), and the constitutive promoter GOS2 from Zea mays. Other promoters include root preferred promoters, such as the maize NAS2 promoter, the maize Cyclo promoter (US Publication No. 2006/0156439, published Jul. 13, 2006), the maize ROOTMET2 promoter (WO 2005/063998, published Jul. 14, 2005), the CRIBIO promoter (WO 2006/055487, published May 26, 2006), the CRWAQ81 promoter (WO 2005/035770, published Apr. 21, 2005) and the maize ZRP2.47 promoter (NCBI Accession No. U38790; NCBI GI No. 1063664).

Recombinant DNA constructs of the present disclosure may also include other regulatory elements including, but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8:4395-4405 (1988); Callis et al., Genes Dev. 1:1183-1200 (1987)).

An enhancer or enhancer element refers to a cis-acting transcriptional regulatory element, a.k.a. cis-element, which confers an aspect of the overall expression pattern, but is usually insufficient alone to drive transcription, of an operably linked polynucleotide sequence. An isolated enhancer element may be fused to a promoter to produce a chimeric promoter cis-element, which confers an aspect of the overall modulation of gene expression. Enhancers are known in the art and include the SV40 enhancer region, the CaMV 35S enhancer element, and the like. Some enhancers are also known to alter normal regulatory element expression patterns, for example, by causing a regulatory element to be expressed constitutively when without the enhancer, the same regulatory element is expressed only in one specific tissue or a few specific tissues. Duplicating the upstream region of the CaMV35S promoter has been shown to increase expression by approximately tenfold (Kay, R. et al., (1987) Science 236:1299-1302).

### Compositions

Provided are plants comprising in their genome any of the recombinant DNA constructs of the present disclosure (such as any of the constructs discussed above). In certain embodiments, the recombinant DNA constructs comprise heterologous regulatory elements. Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct. These seeds can be grown to produce plants that would exhibit an altered agronomic characteristic or used in a breeding program to produce hybrid seed, which can be grown to produce plants that would exhibit such an altered agronomic characteristic. The seeds may be maize seeds, wheat seeds, or rice seeds.

The plant of the compositions described herein may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley or millet.

The recombinant DNA construct is stably integrated into the genome of the plant.

Embodiments include but are not limited to the following:
1. A transgenic plant (for example, a rice, maize, wheat or soybean plant) comprising in its genome a recombinant DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 3, 6, 9, 28, 30, 32, 34, 37, 39, 41, 43, 46, 48, 50 or 52; and wherein the plant exhibits sterile trait.
2. The plant of embodiment 1, wherein the polynucleotide encodes ZOS3-17, Dnak or PPT1-1 polypeptide, for example from Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Cicer arietinum, Solanum tuberosum, Brassica oleracea, Zea mays, Glycine max, Glycine tabacina, Glycine soja or Glycine tomentella, or wheat.
3. Any progeny of the above plants in embodiments 1 to 2, any seeds of the above plants in embodiments 1 to 2, any seeds of progeny of the above plants in embodiments 1 to 2, and cells from any of the above plants in embodiments 1 to 2 and progeny thereof.

In any of the foregoing embodiments 1 to 3 or any other embodiments of the present disclosure, the recombinant DNA construct may comprise at least one heterologous promoter functional in a plant as a regulatory element.

The examples below describe some representative protocols and techniques for regulating plant sterile trait and observing and/or evaluating plants agricultural characteristics under such conditions.
1. Progeny of a transformed plant which is hemizygous with respect to a recombinant DNA construct, such that the progeny is segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).
2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).
3. Two hybrid lines, where the first hybrid line is produced from two parent inbred lines, and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).
4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% identity of nuclear genetic material compared to the plant comprising the recombinant DNA construct.

### Methods

Methods are provided for genome modification of a target sequence in the genome of a plant or plant cell, for selecting plants, for gene editing, and for inserting a polynucleotide of interest into the genome of a plant. The methods employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest.

In one embodiment, a method for modifying a target site in the genome of a plant cell, comprises introducing a guide RNA and a Cas endonuclease into said plant, wherein said guide RNA and Cas endonuclease are capable of forming a complex that enables the Cas endonuclease to introduce a double strand break at said target site.

Further provided is a method for modifying a target site in the genome of a plant cell, the method comprising: a) introducing into a plant cell a guide RNA and a Cas endonuclease, wherein said guide RNA and Cas endonuclease are capable of forming a complex that enables the Cas endonuclease to introduce a double strand break at said target site; and, b) identifying at least one plant cell that has a modification at said target site, wherein the modification includes at least one deletion, insertion or substitution of one or more nucleotides in said target site.

Proteins may be altered in various ways including amino acid substitution, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

Also provided is a method for editing a nucleotide sequence in the genome of a cell, the method comprising introducing a guide polynucleotide, a Cas endonuclease, and optionally a polynucleotide modification template, into a cell, wherein said guide RNA and Cas endonuclease are capable of forming a complex that enables the Cas endonuclease to introduce a double strand break at target site in the genome at said cell, wherein said polynucleotide modification template comprises at least one nucleotide modification of said nucleotide sequence. The nucleotide sequence in the genome of a cell is selected from the group consisting of a promoter sequence, a terminator sequence, a regulatory element sequence, a splice site, a coding sequence, a polyubiquitination site, an intron site and an intron enhancing motif.

Further provided is a method for editing a promoter sequence in the genome of a cell, the methods comprising introducing a guide polynucleotide, a polynucleotide modification template and at least one Cas endonuclease into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

A method for producing a transgenic plant comprising transforming a plant cell with any of the isolated polynucleotides or recombinant DNA constructs of the present disclosure and regenerating a transgenic plant from the transformed plant cell. The disclosure is also directed to the transgenic plant produced by this method, and transgenic seed obtained from this transgenic plant.

A method for isolating a polypeptide of the disclosure from a cell or culture medium of the cell, wherein the cell comprises a recombinant DNA construct comprising a polynucleotide of the disclosure operably linked to at least one regulatory element, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method for producing a modified plant comprising transforming a plant cell with any of the CRISPR-Cas construct of the present disclosure and regenerating a modified plant from the transformed plant cell, wherein, the modified plant and the modified seed obtained by this method may be used in other methods of the present disclosure.

A method for altering the expression level of a polypeptide of the disclosure in a plant comprising: (a) transforming a regenerable plant cell with a CRISPR-Cas construct of the present disclosure; and (b) regenerating a modified plant from the regenerable plant cell after step (a), wherein the plant gene were edited; and (c) growing the transformed plant, wherein the expression of the CRISPR-Cas construct results in production of altered levels of the polypeptide of the disclosure in the transformed plant.

A method of producing seed comprising any of the preceding methods, and further comprising obtaining seeds from said progeny plant, wherein said seeds comprise in their genome said recombinant DNA construct.

One embodiment provides a method for making sterile plant (e.g., rice), the method comprising increasing the expression of a polynucleotide encoding a polypeptide having at least 90% sequence identity to SEQ ID NO: 3, 6, or 9. The increase in expression of the polynucleotide may be mediated by any of the methods described herein using any of the polynucleotides or compositions described herein.

In some embodiments, the disclosure provides seeds that comprise in their genome the recombinant DNA construct of the disclosure.

The introduction of recombinant DNA constructs of the present disclosure into plants may be carried out by any suitable technique, including but not limited to direct DNA uptake, chemical treatment, electroporation, microinjection, cell fusion, infection, vector mediated DNA transfer, bombardment, or Agrobacterium mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### Stacking of Traits

Modified plants may comprise a stack of one or more sterile polynucleotides disclosed herein with one or more additional polynucleotides resulting in the production or suppression of multiple polypeptide sequences. Modified plants comprising stacks of polynucleotide sequences can be obtained by either or both of traditional breeding methods or through genetic engineering methods. These methods include, but are not limited to, breeding individual lines each comprising a polynucleotide of interest, genome editing, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Expression of the sequences can be driven by the same promoter or by different promoters. In certain cases, it may be desirable to introduce a transformation cassette that will suppress the expression of the polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system.

### EXAMPLES

### Example 1

### Sterile Genes Cloning and Over-Expression Vectors Construction

A binary construct that contains four multimerized enhancers elements derived from the Cauliflower Mosaic Virus 35S (CaMV 35S) promoter was used, and the rice activation tagging population was developed from four japonica (Oryza sativa ssp. Japonica) varieties (Zhonghua 11, Chaoyou 1, Taizhong 65 and Nipponbare), which were transformed by Agrobacteria-mediated transformation method as described by Lin and Zhang ((2005) Plant Cell Rep. 23:540-547). The transgenic lines generated were developed and the transgenic seeds were harvested to form the rice activation tagging population.

Sterile tagging lines (ATLs) were confirmed in repeated field experiments and their T-DNA insertion loci were determined. The genes near by the left border and right border of the T-DNA were cloned and the functional genes were recapitulated by field screens. Only the recapitulated functional genes are showed herein. And based on LOC IDs of these genes shown in Table 2. Primers were designed for cloning rice sterile genes OsZOS3-17, OsDnaK and OsPPT1-1.

TABLE 2
Rice gene names, Gene IDs (from TIGR) and Construct IDs

| Gene name | LOC ID | Construct ID |
|---|---|---|
| OsZOS3-17 | LOC_Os03g50850 | DP0640 |
| OsDnaK | LOC_Os03g16920 | DP1673 |
| OsPPT1-1 | LOC_Os08g43540 | DP1675 |

PCR amplified products were extracted after the agarose gel electrophoresis using a column kit and then ligated with TA cloning vectors. The sequences and orientation in these constructs were confirmed by sequencing. Each gene was cloned into a plant binary construct.

### Example 2

Generation of Rice Plants with Increased Gene Expression

The over-expression vectors and empty vector (DP0158) were transformed into Zhonghua 11 (Oryza sativa L.) by Agrobacteria-mediated as described by Lin and Zhang (Plant Cell Rep. 23:540-547 (2005)). The transgenic seedlings (TO) generated in transformation laboratory were transplanted in the field to get T1 seeds. The T1 and T2 seeds were stored at 4 °C. The over-expression vectors contain DsRED and HYG genes. T1 and T2 seeds which showed red color under green fluorescent light were transgenic seeds and were used in the following sterility assays.

### Gene Expression Analysis in Transgenic Rice Plants:

Gene expression levels in the transgenic rice plants were analyzed by a standard real-time RT-PCR procedure. EF1α gene was used as an internal control to show that the amplification and loading of samples from the transgenic rice and control plant were similar. The expression level was normalized based on the EF1α mRNA levels.

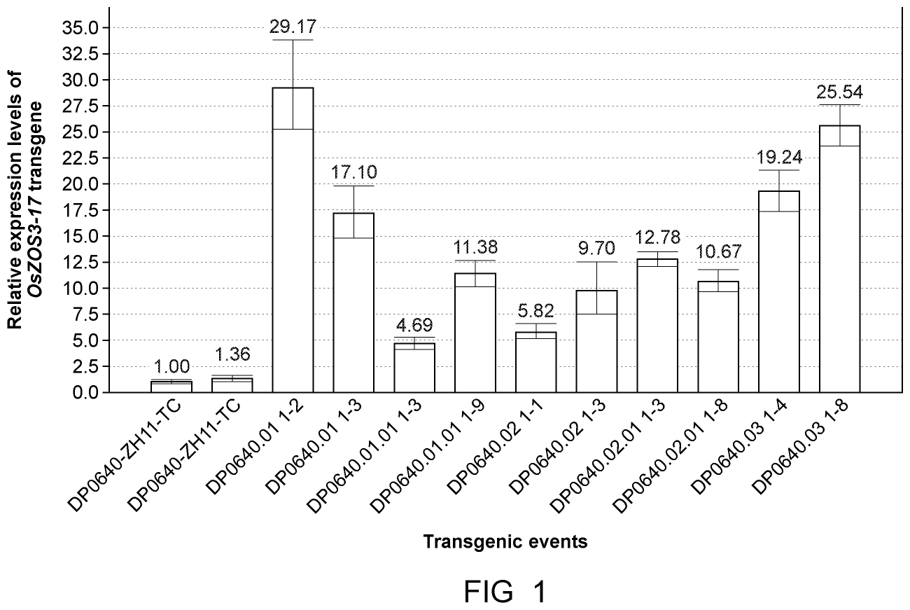

OsZOS3-17 gene expression levels in the DP0640 transgenic rice plants were detected using the primers of SEQ ID NOs: 16 and 17. mRNA was extracted from leaf of T1 or T2 generation seedlings. As shown in FIG. 1, the expression level in ZH11-TC rice is set at 1.00, and OsZOS3-17 gene is over-expressed in almost all the tested transgenic lines.

DP0640-F1
(SEQ ID NO: 16)
5′-AGGCAAAAGAACTCTGGGAG-3′
DP0640-R1
(SEQ ID NO: 17)
5′-CTGCAGATCAGTGTAGGTCTTC-3′

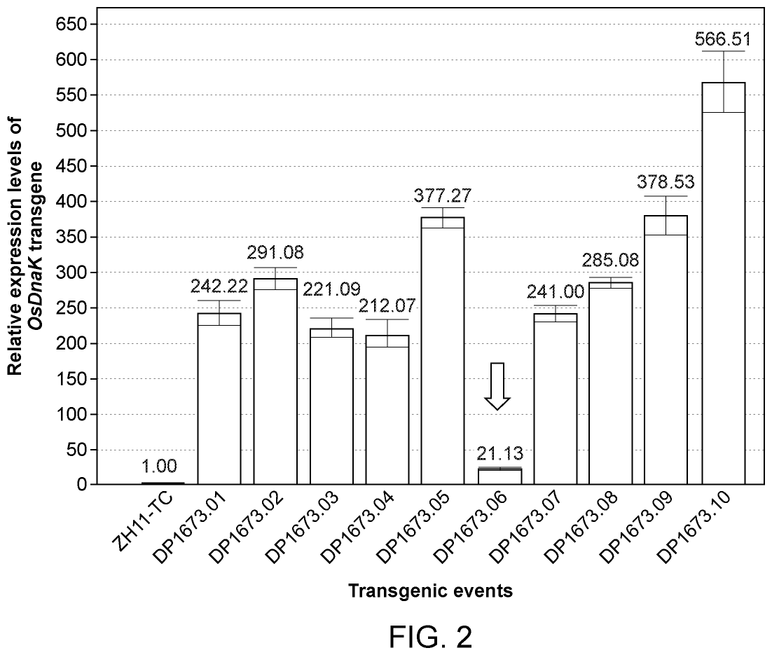

OsDnaK gene expression levels in the DP1673 transgenic rice plants were detected using the primers of SEQ ID NOs: 18 and 19. mRNA was extracted from leaf of T1 generation seedlings. As shown in FIG. 2, the expression level in ZH11-TC rice is set at 1.00, and OsDnaK gene is over-expressed in almost all the tested transgenic lines.

DP1673-F1
(SEQ ID NO: 18)
ATCGAGGATGCCATCAAGTG
DP1673-R1
(SEQ ID NO: 19)
CGCCCTGGTACATCTTTGAG

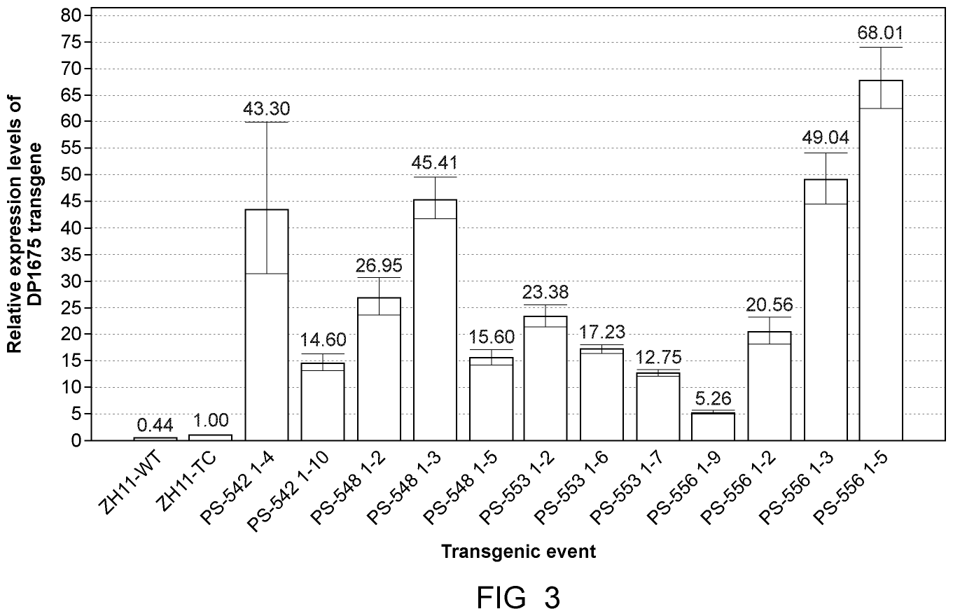

OsPPT1-1 gene expression levels in the DP1675 transgenic rice plants were detected using the primers of SEQ ID NOs: 20 and 21. mRNA was extracted from leaf of T1 generation seedlings. As shown in FIG. 3, the expression level in ZH11-TC rice is set at 1.00, and OsPPT1-1 gene is over-expressed in almost all the tested transgenic lines.

DP1675-F1
(SEQ ID NO: 20)
5′-GTGGCAAGAACATCGAGATTG-3′
DP1675-R1
(SEQ ID NO: 21)
5′-TTCAATCTCAGCCACGTACTC-3′

### Example 3

### Sterile Trait of OsZOS3-17 Over-Expression Rice Plants

The sterility phenotype was observed during propagation. The fertility was divided into three levels according to pollen fertility rate and seed setting rate as shown in Table 3. The pollen is classified into four types by pollen microscope examination and reaction to 12-KI solution as shown in Table 4.

TABLE 3
Classification of plant fertility by pollen
fertility and seed setting rate

| | Classification | | |
|---|---|---|---|
| Parameter | Sterility | Semi-sterility | Fertility |
| Pollen fertility (%) | <20 | 20-80 | >80 |
| Seed setting rate (%) | <20 | 20-80 | >80 |

TABLE 4
Pollen classification by pollen microscope examination
and reaction to I₂-KI solution

| | Type | | | |
|---|---|---|---|---|
| | Typical | Spherical | Stained | |
| Character | abortion type | abortion type | abortion type | Fertility |
| Stage | Monokaryotic | Dikaryophase | Trinucleate | |
| | phase | | stage | |
| Pollen | Anomaly | Round | Round | Round |
| particle | | | | and full |
| shape | | | | |
| Dyeing | Non-staining | Non-staining | Partial and | Deep and |
| condition | | | light staining | even |
| (I₂-KI) | | | | staining |

The OsZOS3-17 transgenic rice plants were planted in Beijing (40°13′N) to get seeds. The results were as below.

### Sterile Trait at T1 Generation

The seed setting rates of OsZOS3-17 transgenic rice lines at T1 generation are listed in Table 5. Four of thirteen lines (DP0640.05, DP0640.06, DP0640.07, DP0640.13) were fertile. In the other nine lines, there are fertile plants and sterile plants. The ratio of fertile plants to sterile plants in all lines was about 3:1, indicating a potential recessive sterile gene.

TABLE 5
Seed setting of DP0640 at T1 generation in Beijing (1^{st} experiment)

| | Total | Seed setting | | |
|---|---|---|---|---|
| | plant | | Semi- | |
| Line ID | number | Fertile | sterile | Sterile |
| DP0640.01 | 10 | 4 | 0 | 6 |
| DP0640.02 | 10 | 8 | 0 | 2 |
| DP0640.03 | 10 | 6 | 0 | 4 |
| DP0640.05 | 10 | 10 | 0 | 0 |
| DP0640.06 | 10 | 10 | 0 | 0 |
| DP0640.07 | 10 | 10 | 0 | 0 |
| DP0640.09 | 10 | 7 | 0 | 3 |
| DP0640.10 | 10 | 6 | 0 | 4 |
| DP0640.12 | 6 | 4 | 0 | 2 |
| DP0640.13 | 10 | 10 | 0 | 0 |
| DP0640.16 | 10 | 4 | 0 | 6 |
| DP0640.17 | 5 | 3 | 1 | 1 |
| DP0640.18 | 3 | 2 | 0 | 1 |
| Totally | 114 | 84 | 1 | 29 |

To confirm the observation, three lines at T1 generation were planted again in Beijing. The pollen from the transgenic rice plants were tested, and the same panicles were bagged to measure the seed setting rate. As shown in table 6, the pollen from all the three lines at T1 generation were fertile. As shown in table 7, the bagged panicles from the three OsZOS3-17 transgenic rice lines were fertile, semi-sterile and sterile; and the ratio of fertile panicles to sterile panicles was 2.4:1. As shown in table 8, the three OsZOS3-17 transgenic rice lines were fertile, semi-sterile and sterile panicles; the ratio of fertile panicles to sterile panicles was about 3:1. These results consistently demonstrate that OsZOS3-17 is likely a recessive sterile gene.

TABLE 6
Pollen fertility of DP0640 at T1 generation
in Beijing (2^{nd} experiment)

| | Panicle | Pollen fertility | | |
|---|---|---|---|---|
| | number | | Semi- | |
| Line ID | tested | Fertility | sterility | Sterility |
| DP0640.01 | 10 | 10 | 0 | 0 |
| DP0640.02 | 10 | 10 | 0 | 0 |
| DP0640.03 | 9 | 9 | 0 | 0 |
| Totally | 29 | 29 | 0 | 0 |

TABLE 7
Seed setting of bagged panicles of DP0640
at T1 generation in Beijing (2^{nd} experiment)

| | Bagged | | | |
|---|---|---|---|---|
| | panicle | Seed setting | | |
| Line ID | number | Fertility | Semi-sterility | Sterility |
| DP0640.01 | 8 | 5 | 0 | 3 |
| DP0640.02 | 10 | 8 | 0 | 2 |
| DP0640.03 | 8 | 4 | 2 | 2 |
| Totally | 26 | 17 | 2 | 7 |

TABLE 8
Seed setting of DP0640 at T1 generation
in Beijing (2^{nd} experiment)

| | | Seed setting | | |
|---|---|---|---|---|
| | Plant | | Semi- | |
| Line ID | number | Fertility | sterility | Sterility |
| DP0640.01 | 15 | 9 | 1 | 5 |
| DP0640.02 | 15 | 11 | 1 | 3 |
| DP0640.03 | 10 | 8 | 0 | 2 |
| Totally | 40 | 28 | 2 | 10 |

### Sterile Trait at T2 Generation

OsZOS3-17 transgenic rice plants at T2 generation were planted. The pollen from the transgenic rice plants were tested, and the same panicles were bagged to measure the seed setting rate. As shown in table 9, the pollen from all the 13 lines were fertile. As shown in table 10, the bagged panicles from the OsZOS3-17 transgenic rice lines were fertile, semi-sterile and sterile; and the ratio of fertile panicles to sterile panicles was 3.9:1. As shown in table 11, the thirteen OsZOS3-17 transgenic rice lines were fertile, semi-sterile and sterile panicles; the ratio of fertile panicles to sterile panicles was 3.5:1. These results are consistent with the results of T1 generation, and it shows that OsZOS3-17 is likely a recessive sterile gene.

TABLE 9
Pollen fertility of DP0640 at T2 generation in Beijing

| | Panicle | Pollen fertility | | |
|---|---|---|---|---|
| | number | | Semi- | |
| Line ID | tested | Fertility | sterility | Sterility |
| DP0640.01.01 | 9 | 9 | 0 | 0 |
| DP0640.02.01 | 10 | 10 | 0 | 0 |
| DP0640.03.01 | 9 | 9 | 0 | 0 |
| DP0640.05.01 | 9 | 9 | 0 | 0 |
| DP0640.06.01 | 10 | 10 | 0 | 0 |
| DP0640.07.01 | 10 | 10 | 0 | 0 |
| DP0640.09.01 | 10 | 10 | 0 | 0 |
| DP0640.10.01 | 10 | 10 | 0 | 0 |
| DP0640.12.01 | 9 | 9 | 0 | 0 |
| DP0640.13.01 | 10 | 10 | 0 | 0 |
| DP0640.16.01 | 10 | 10 | 0 | 0 |
| DP0640.17.01 | 10 | 10 | 0 | 0 |
| DP0640.18.01 | 10 | 10 | 0 | 0 |
| Totally | 126 | 126 | 0 | 0 |

TABLE 10
Seed setting of bagged panicles of
DP0640 at T2 generation in Beijing

| | Bagged | Seed setting | | |
|---|---|---|---|---|
| | panicle | | Semi- | |
| Line ID | number | Fertility | sterility | Sterility |
| DP0640.01.01 | 9 | 5 | 2 | 2 |
| DP0640.02.01 | 10 | 5 | 2 | 3 |
| DP0640.03.01 | 9 | 4 | 3 | 2 |
| DP0640.05.01 | 10 | 5 | 4 | 1 |
| DP0640.06.01 | 10 | 6 | 2 | 2 |
| DP0640.07.01 | 9 | 7 | 0 | 2 |
| DP0640.09.01 | 10 | 6 | 2 | 2 |
| DP0640.10.01 | 10 | 7 | 1 | 2 |
| DP0640.12.01 | 9 | 6 | 1 | 2 |
| DP0640.13.01 | 10 | 9 | 1 | 0 |
| DP0640.16.01 | 9 | 5 | 1 | 3 |
| DP0640.17.01 | 9 | 7 | 2 | 0 |
| DP0640.18.01 | 9 | 9 | 0 | 0 |
| Totally | 123 | 81 | 21 | 21 |

TABLE 11
Seed setting of DP0640 at T2 generation in Beijing

| | | Seed setting | | |
|---|---|---|---|---|
| | Plant | | Semi- | |
| Line ID | number | Fertility | sterility | Sterility |
| DP0640.01.01 | 19 | 14 | 1 | 4 |
| DP0640.02.01 | 20 | 15 | 1 | 4 |
| DP0640.03.01 | 19 | 15 | 0 | 4 |
| DP0640.05.01 | 18 | 12 | 5 | 1 |
| DP0640.06.01 | 20 | 16 | 2 | 2 |
| DP0640.07.01 | 20 | 12 | 0 | 8 |
| DP0640.09.01 | 15 | 9 | 2 | 4 |
| DP0640.10.01 | 20 | 13 | 2 | 5 |
| DP0640.12.01 | 20 | 13 | 1 | 6 |
| DP0640.13.01 | 20 | 20 | 0 | 0 |
| DP0640.16.01 | 20 | 9 | 0 | 11 |
| DP0640.17.01 | 20 | 17 | 2 | 1 |
| DP0640.18.01 | 20 | 16 | 3 | 1 |
| Totally | 251 | 181 | 19 | 51 |

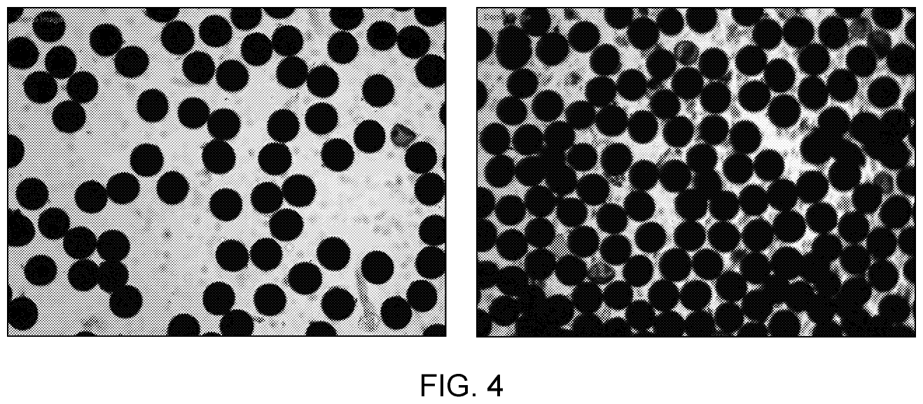

Both pollen and pistil phenotypes were observed in the above experiments. The pollen of OsZOS3-17 transgenic rice plants were fertile (FIG. 4). And no abnormal phenotype in pistils was observed with OsZOS3-17 transgenic rice plants. During the field experiments, except a few individual plants, most of the transgenic rice plants had only a sterile phenotype, and the other phenotypes, include but not limited to, plant height, growth period, yield etc., were normal.

### OsZOS3-17 Transgenic Rice Seed Phenotype

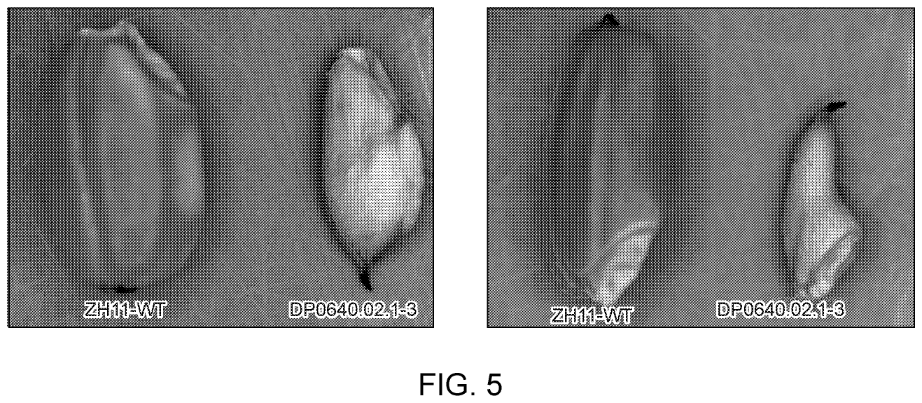

One line of DP0640.02 at T2 generation and one line of DP0640.12.01 at T3 generation were planted to get seeds. However, most of the seeds were shrunken grain (FIG. 5). The seed germination experiment is needed to validate the sterility phenotype of shrunken grains.

### Seed Germination Experiment of OsZOS3-17 Transgenic Rice Seeds

One T2 generation line of DP0640.02 and one T3 generation line of DP0640.12.01 were tested 3 times with 50 seeds per replication. ZH11-TC and DP0158 are used as controls. All the seeds were put in oven at 42 °C for 72 h to break seed dormancy. Seeds were placed evenly in a germination box, covered with filter paper and 18 ml water, and put into an incubator at 28 °C for 8 days. The resulting germination rate was then recorded.

As shown in Table 12, the germination rate of the seeds from OsZOS3-17 transgenic rice plants are lower than that of ZH11-TC and DP0158 controls. And the germination rate of the homozygosis seeds is lower than that of the heterozygosis seeds in OsZOS3-17 transgenic rice plants. All the shrunken seeds did not germinate.

TABLE 12
Seed germination rate of DP0640 at T2 and T3 generations

| | | Totally | | Un- | Germi- |
|---|---|---|---|---|---|
| | | seed | Germi- | germi- | nation |
| Line ID | Genotype | number | nated | nated | rate |
| ZH11-TC | | 150 | 147 | 3 | 98% |
| DP0158.mix | | 150 | 142 | 8 | 95% |
| DP0640.02-1 | Heterozygous | 150 | 111 | 39 | 74% |
| DP0640.02-2 | Homozygous | 150 | 8 | 142 | 5% |
| DP0640.12.01-1 | Heterozygous | 150 | 136 | 14 | 91% |
| DP0640.12.01-2 | Homozygous | 150 | 7 | 143 | 5% |

### Fluorescence Segregation Ratio of OsZOS3-17 Transgenic Rice Seeds

The fluorescence segregation ratio of OsZOS3-17 transgenic rice seeds were measured. The seeds of 28 plants from three lines at T1 generation were detected, and 100 seeds from each plant. The results demonstrated that 2128 of 2800 seeds show red color under green fluorescence light, while 672 of 2800 seeds didn't show red color. The ratio of red color seeds to normal seeds was 3.2:1.

The seeds of 156 plants from 13 lines at T2 generation were inspected also, using 100 seeds from each plant. A total of 15519 seeds were tested. The results demonstrated that 11836 seeds show red color under green fluorescence light, while 3683 seeds didn't show red color. The ratio of red color seeds to normal seeds was 3.2:1. These results indicated that single OsZOS3-17 gene was inserted in the OsZOS3-17 transgenic rice genome.

TABLE 13
Fluorescence segregation ratio of OsZOS3-17 transgenic rice seeds

| | Total seed | DsRed-seed | DsRed-null | |
|---|---|---|---|---|
| Generation | number | number | seed number | Ratio |
| T1 | 2800 | 2128 | 672 | 3.17 |
| T2 | 15519 | 11836 | 3683 | 3.21 |

### Cross-In and Cross-Out Results of OsZOS3-17 Transgenic Rice

To further investigate the function of OsZOS3-17 gene, cross-in and cross-out experiments were performed, and only the homozygous plants were used in the experiments. As shown in table 14, the cross-in of 560 florets from 10 plants with pollen from wildtype ZH11 did not produce any seed; while the cross-out of 705 florets with wildtype ZH11 seeds to OsZOS3-17 transgenic plants produce seeds, and some seeds show red color under the green florescence light. This data indicate OsZOS3-17 is a potential female sterile gene.

TABLE 14
Seed set of cross-In and cross-Out of DP0640 transgenic lines

| | | Cross-breeding | | | Seed number | | |
|---|---|---|---|---|---|---|---|
| | | (♀) | (♂) | Floret | | DsRed | Null |
| Line ID | Plant ID | Cross-In | Cross-Out | number | Total | seed | seed |
| DP0640.02.1-3 | 1-1 | ✓ | | 51 | 0 | 0 | 0 |
| DP0640.02.1-3 | 1-2 | ✓ | | 83 | 0 | 0 | 0 |
| DP0640.02.1-3 | 2-1 | ✓ | | 51 | 0 | 0 | 0 |
| DP0640.02.1-3 | 1-7 | ✓ | | 49 | 0 | 0 | 0 |
| DP0640.02.1-3 | 1-8 | ✓ | | 48 | 0 | 0 | 0 |
| DP0640.02.2-4 | 1-8 | ✓ | | 51 | 0 | 0 | 0 |
| DP0640.02.2-4 | 2-4 | ✓ | | 54 | 0 | 0 | 0 |
| DP0640.02.2-4 | 1-1 | ✓ | | 54 | 0 | 0 | 0 |
| DP0640.02.2-4 | 1-2 | ✓ | | 55 | 0 | 0 | 0 |
| DP0640.02.2-4 | 1-5 | ✓ | | 64 | 0 | 0 | 0 |
| Totally | | | | 560 | 0 | 0 | 0 |
| DP0640.02.1-3 | 1-3 | | ✓ | 79 | 14 | 13 | 1 |
| DP0640.02.1-3 | 1-5 | | ✓ | 75 | 6 | 0 | 6 |
| DP0640.02.1-3 | 2-3 | | ✓ | 110 | 51 | 34 | 17 |
| DP0640.02.1-3 | 1-2 | | ✓ | 74 | 41 | 19 | 22 |
| DP0640.02.1-3 | 1-3 | | ✓ | 83 | 35 | 32 | 3 |
| DP0640.02.1-3 | 1-4 | | ✓ | 86 | 12 | 4 | 8 |
| DP0640.02.1-3 | 2-8 | | ✓ | 60 | 25 | 15 | 10 |
| DP0640.02.2-4 | 1-1 | | ✓ | 92 | 22 | 8 | 14 |
| DP0640.02.2-4 | 1-8 | | ✓ | 46 | 3 | 0 | 3 |
| Totally | | | | 705 | 209 | 125 | 84 |

The cross-out seeds were validated by PCR with the primers of SEQ ID NO: 22 and SEQ ID NO: 23. The PCR validation results demonstrated that all the cross-out plants contained the OsZOS3-17 gene. The seed setting of the cross-out plants was also measured. As shown in table 15, all the plants were fertile.

TABLE 15
Sees sets of cross-out of DP0640 lines

| | | Total | Seed setting | | |
|---|---|---|---|---|---|
| | Gener- | plant | | Semi- | |
| Line ID | ation | number | Fertility | sterility | Sterility |
| DP0640.01♂.1-9-F | F1 | 10 | 10 | 0 | 0 |
| DP0640.01.01♂.1-7-F | F1 | 10 | 10 | 0 | 0 |
| DP0640.02.01♂.1-1-F | F1 | 10 | 10 | 0 | 0 |
| DP0640.07.01♂.1-9-F | F1 | 4 | 4 | 0 | 0 |
| DP0640.12.01♂.1-3-F | F1 | 5 | 4 | 1 | 0 |
| DP0640.10.01♂.1-10-F | F1 | 2 | 2 | 0 | 0 |

### Example 4

### Sterile Trait of OsDnak Over-Expression Rice Plants

The classification of plant fertility and pollen types are illustrated in example 3. The OsDnaK transgenic rice plants were planted to get seeds. The results are shown below.

### Phenotype at T0 Generation

Forty-nine transgenic seedlings (TO, DP1673) generated in the transformation laboratory were transplanted in Hainan field (18°30′N) to get T1 seeds in October. The rice plants were managed by normal practice using pesticides and fertilizers. The architecture, panicle phenotype of OsDnaK transgenic rice plants were same to that of wild-type rice plants during the whole growth period. Finally, some seeds were harvested from 25 lines and no seed was obtained from the remaining 24 lines. This data indicates that the overexpression of OsDnaK gene may impact the sterile trait, and OsDnaK may be a dominant sterile gene.

### Sterile Trait at T1 Generation

To further confirm the sterility trait observation at the TO generation, the seeds from the first 15 lines were planted in Beijing field (40°13′N). The seed setting rates or the pollen fertility of OsDnaK transgenic rice plants were measured. The seed setting rates of DP1673 T1 lines are listed in Table 16. Thirteen of fifteen lines showed a complete sterile phenotype and two lines (DP1673.06, DP1673.12) were fertile. This data further indicates overexpression of OsDnak gene may impact the sterile trait.

TABLE 16
Sterile phenotypes of DP1673 at T1 generation in Beijing

| | Total | Seed set | | |
|---|---|---|---|---|
| | plant | | Semi- | |
| Line ID | number | Fertility | sterility | Sterility |
| DP1673.01 | 10 | 0 | 0 | 10 |
| DP1673.02 | 10 | 0 | 0 | 10 |
| DP1673.03 | 10 | 0 | 0 | 10 |
| DP1673.04 | 10 | 0 | 0 | 10 |
| DP1673.05 | 5 | 0 | 0 | 5 |
| DP1673.06 | 10 | 8 | 0 | 2 |
| DP1673.07 | 10 | 0 | 0 | 10 |
| DP1673.08 | 10 | 0 | 0 | 10 |
| DP1673.09 | 8 | 0 | 0 | 8 |
| DP1673.10 | 10 | 0 | 0 | 10 |
| DP1673.11 | 10 | 0 | 0 | 10 |
| DP1673.12 | 10 | 10 | 0 | 0 |
| DP1673.13 | 10 | 1 | 0 | 9 |
| DP1673.14 | 10 | 0 | 0 | 10 |
| DP1673.15 | 10 | 0 | 0 | 10 |

To understand if the phenotypes were impacted by locations and/or environmental factors, the stubbles of OsDnaK transgenic rice were transferred from Beijing to Hainan in October. As indicated in Table 17, the pollen from almost all the rice plants are fertile or semi-sterile. While the seed sets of 6 lines (DP1673.01, DP1673.02, DP1673.05, DP1673.10, DP1673.14, DP1673.15) remained completely sterile at both Beijing and Hainan; another 6 lines (DP1673.03, DP1673.04, DP1673.07, DP1673.08, DP1673.09, DP1673.11) were sterile in Beijing, but set seeds in Hainan (semi or high sterile); and the rest 3 lines (DP1673.06, DP1673.12, DP1673.13) showed segregating sterile phenotypes at both Beijing and Hainan.

TABLE 17
Sterile phenotypes of DP1673 at T1 generation in Hainan

| | Total | Pollen fertility | | | Seed set | | |
|---|---|---|---|---|---|---|---|
| | plant | | Semi- | | | Semi- | |
| Line ID | number | Fertility | sterility | Sterility | Fertility | sterility | Sterility |
| DP1673.01 | 10 | 9 | 1 | 0 | 0 | 0 | 10 |
| DP1673.02 | 9 | 4 | 5 | 0 | 0 | 0 | 9 |
| DP1673.03 | 10 | 3 | 7 | 0 | 0 | 0 | 10 |
| DP1673.04 | 10 | 3 | 7 | 0 | 0 | 1 | 9 |
| DP1673.05 | 6 | 5 | 0 | 1 | 0 | 0 | 6 |
| DP1673.06 | 8 | 8 | 0 | 0 | 6 | 2 | 0 |
| DP1673.07 | 10 | 8 | 2 | 0 | 0 | 1 | 9 |
| DP1673.08 | 9 | 7 | 2 | 0 | 0 | 0 | 9 |
| DP1673.09 | 7 | 5 | 0 | 2 | 0 | 1 | 6 |
| DP1673.10 | 6 | 6 | 0 | 0 | 0 | 0 | 6 |
| DP1673.11 | 5 | 3 | 2 | 0 | 0 | 3 | 2 |
| DP1673.12 | 6 | 3 | 3 | 0 | 0 | 0 | 6 |
| DP1673.13 | 8 | 5 | 3 | 0 | 0 | 1 | 7 |
| DP1673.14 | 5 | 3 | 2 | 0 | 0 | 0 | 5 |
| DP1673.15 | 3 | 3 | 0 | 0 | 0 | 0 | 3 |

Interestingly, seed sets were different in panicles from same plant in 6 lines (DP1673.03, DP1673.04, DP1673.07, DP1673.08, DP1673.11, DP1673.12). The young tiller-developed panicles showed fertile phenotypes, whereas the primary and older panicles showed sterile phenotypes under both bagged or un-bagged conditions.

The temperature and day-light-length during the panicle development were different between Beijing and Hainan. In Beijing field, the temperature was more than 23 °C and day-light-length was longer than 12 hours; whereas the temperature was less than 23 °C and day-light-length was shorter than 12 hours in Hainan field during the young panicle development. These results indicate that the sterility of DP1673 plants was impacted by temperature and day-light-length.

To understand if the sterile phenotypes of these stubbles were retained as in the first Beijing field planting, the stubbles from Hainan were transferred back to Beijing field. As indicated in Table 18, all the events showed sterile phenotypes except DP1673.06 line as observed in the first planting. These results further confirmed OsDnaK is likely a female-sterile gene and the function was impacted by the environmental factors.

TABLE 18
Sterile phenotypes of DP1673 at T1
generation in Beijing (2^{nd} time)

| | Total | Seed set | | |
|---|---|---|---|---|
| | plant | | Semi- | |
| Line ID | number | Fertility | sterility | Sterility |
| DP1673.01 | 5 | 0 | 0 | 5 |
| DP1673.02 | 5 | 0 | 0 | 5 |
| DP1673.03 | 5 | 0 | 0 | 5 |
| DP1673.04 | 8 | 0 | 0 | 8 |
| DP1673.05 | 3 | 0 | 0 | 3 |
| DP1673.06 | 3 | 2 | 0 | 1 |
| DP1673.07 | 4 | 0 | 0 | 4 |
| DP1673.08 | 6 | 0 | 0 | 6 |
| DP1673.09 | 4 | 0 | 0 | 4 |
| DP1673.10 | 3 | 0 | 0 | 3 |
| DP1673.11 | 2 | 0 | 0 | 2 |
| DP1673.12 | 4 | 0 | 0 | 4 |
| DP1673.13 | 4 | 0 | 0 | 4 |
| DP1673.14 | 3 | 0 | 0 | 3 |

To understand if the transgene expression level in DP1673 plants related to the sterile phenotypes, leaf samples were collected from the stubble plants from the second Beijing field planting, and quantitative RT-PCR analyses was performed. As indicated in FIG. 2, all the events which showed sterile phenotypes have increased OsDnak expression, more than 200-fold, whereas the DP1673.06 line which showed fertility only increased 21-fold. These results indicated that the OsDnak transgene expression level is closely related to the sterility.

### Sterile Trait at T2 Generation

We planted 3 lines (DP1673.04, DP1673.07 and DP1673.06) of the T2 generation. The seed set results indicate that plants from both DP1673.04 and DP1673.07 were sterile, whereas DP1673.06 plants were fertile. Also, young tillers from both DP1673.04 and DP1673.07 plants, that experienced a lower temperature (<20 °C) along with short-day-light-length (<12 hours) showed fertile phenotypes. These results were consistent with the data from T1 generation. OsDnaK is likely a female-sterile gene and its function is impacted by environmental factors.

### Cross-In and Cross-Out Results of OsDnak Transgenic Rice

To further investigate the function of OsDnak gene, cross-in and cross-out experiments were performed in Beijing. As shown in table 19, the cross-in of 502 florets of 7 lines with pollen from wildtype ZH11 produced little, if any seed; the cross-out of 384 of 5 lines to wildtype ZH11 produced seeds, and some seed showed red color under the green florescence light. These data indicate OsDnaK is a potential female sterile gene.

TABLE 19
Seed sets of Cross-In and Cross-Out of DP1673 lines

| | Cross-breeding | | | Seed number | | |
|---|---|---|---|---|---|---|
| | (♀) | (♂) | Floret | | DsRed | Null |
| Line ID | Cross-In | Cross-Out | number | Total | seed | seed |
| DP1673.01 | ✓ | | 133 | 0 | 0 | 0 |
| DP1673.07 | ✓ | | 38 | 0 | 0 | 0 |
| DP1673.08 | ✓ | | 82 | 0 | 0 | 0 |
| DP1673.09 | ✓ | | 100 | 0 | 0 | 0 |
| DP1673.11 | ✓ | | 55 | 4 | 0 | 4 |
| DP1673.12 | ✓ | | 33 | 0 | 0 | 0 |
| DP1673.14 | ✓ | | 61 | 0 | 0 | 0 |
| Totally | | | 502 | 4 | 0 | 4 |
| DP1673.01 | | ✓ | 134 | 38 | 24 | 14 |
| DP1673.04 | | ✓ | 91 | 31 | 20 | 11 |
| DP1673.07 | | ✓ | 50 | 23 | 10 | 13 |
| DP1673.09 | | ✓ | 51 | 6 | 4 | 2 |
| DP1673.13 | | ✓ | 58 | 16 | 5 | 11 |
| Totally | | | 384 | 114 | 63 | 51 |

### Pistil Development of OsDnak Transgenic Plants

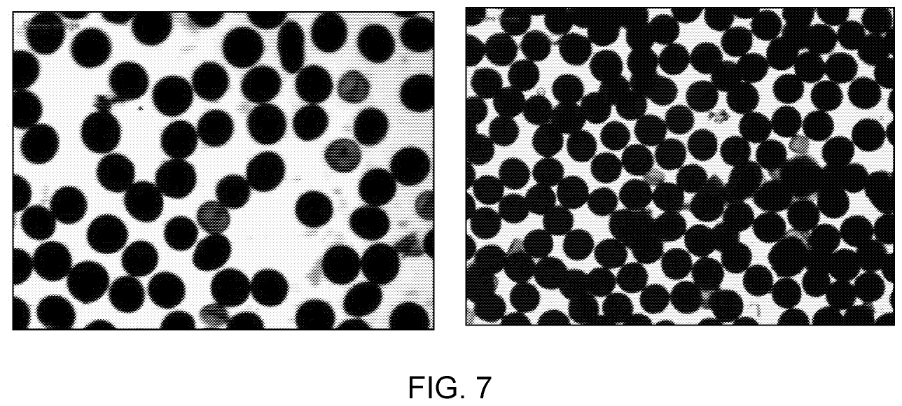

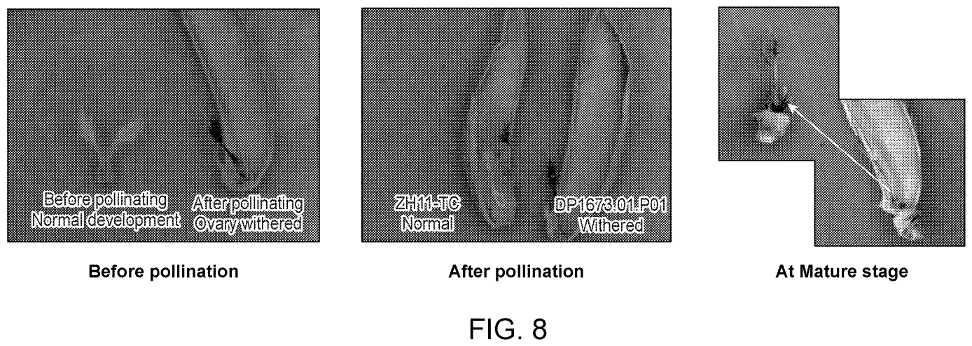

To understand the female sterile mechanism of OsDnak, the development process of DP1673.01 plants at T2 generation were carefully observed in Beijing field. The pollen from DP1673 plants were fertile by I₂K-statining as shown in FIG. 7. The fertility of pollen from DP1673.01 plants was more than 90%. The pistils of the DP1673.01 plants apparently grew normally before pollination, however, their ovary and stigmas withered and died after pollination as indicated in FIG. 8. These results clearly demonstrated that OsDnaK prevented pistil development after pollination. A similar phenomenon was observed with other lines, such as DP1673.04 and DP1673.07.

### Example 5

### Sterile Trait of OsPPT1-1 Over-Expression Rice Plants

The classification of plant fertility and pollen types were illustrated in example 3. The OsPPT1-1 transgenic rice plants were planted to get seeds. The results are shown below.

### Sterile Trait at T1 Generation

Fifteen lines at T1 generation were planted in Beijing field (40°13′N). The seed setting rates of OsPPT1-1 transgenic rice at T1 generation are listed in Table 20. Seven of fifteen lines (DP1675.02, DP1675.04, DP1675.09, DP1675.10, DP1675.11, DP1675.14, DP1657.15) were complete fertile. While there were fertile plants and sterile plants in other eight lines. The ratio of fertile plants to sterile plants of all transgenic plants is about 21:4, indicating a potential recessive sterile gene.

TABLE 20
Seed setting of DP1675 at T1 generation in Beijing (1^{st} experiment)

| Total | |
|---|---|
| plant | Seed setting |

| Line ID | number | Fertile | Sterile |
|---|---|---|---|
| DP1675.01 | 10 | 9 | 1 |
| DP1675.02 | 10 | 10 | 0 |
| DP1675.03 | 10 | 7 | 3 |
| DP1675.04 | 10 | 10 | 0 |
| DP1675.05 | 10 | 6 | 4 |
| DP1675.06 | 10 | 8 | 2 |
| DP1675.07 | 10 | 7 | 3 |
| DP1675.08 | 10 | 5 | 5 |
| DP1675.09 | 10 | 10 | 0 |
| DP1675.10 | 10 | 10 | 0 |
| DP1675.11 | 10 | 10 | 0 |
| DP1675.12 | 10 | 8 | 2 |
| DP1675.13 | 10 | 6 | 4 |
| DP1675.14 | 10 | 10 | 0 |
| DP1675.15 | 10 | 10 | 0 |
| Totally | 150 | 126 | 24 |

To confirm the observation, the fifteen lines at the T1 generation were validated again in the Beijing field. The pollen from the transgenic rice plants was examined using microscopy, and the panicles were bagged to measure the seed setting rate. As shown in table 21, the fertile pollen rate of all the lines at T1 generation were 97%. This indicate the pollen fertility of OsPPT1-1 transgenic plants are normal. As shown in table 22, the bagged panicles from the fifteen OsPPT1-1 transgenic rice plants were fertile, semi-sterile and sterile; and the ratio of fertile panicles to sterile panicles was 3.2:1. Before harvest, the seed setting rate of all individual plant were measured. As shown in table 23, the fifteen OsPPT1-1 transgenic rice lines were fertile, semi-sterile and sterile panicles; the ratio of fertile panicles to sterile panicles was about 3.1:1. These results consistently demonstrate that OsPPT1-1 is likely a recessive sterile gene.

TABLE 21
Pollen fertility of DP1675 at T1 generation
in Beijing (2^{nd} experiment)

| | |
|---|---|
| Bagged | Seed set |

| | panicle | | Semi- |
|---|---|---|---|
| Line ID | number | Fertility | sterility |
| DP1675.01 | 8 | 8 | 0 |
| DP1675.02 | 10 | 10 | 0 |
| DP1675.03 | 8 | 8 | 0 |
| DP1675.04 | 10 | 10 | 0 |
| DP1675.05 | 8 | 8 | 0 |
| DP1675.06 | 10 | 10 | 0 |
| DP1675.07 | 10 | 10 | 0 |
| DP1675.08 | 9 | 9 | 0 |
| DP1675.09 | 10 | 10 | 0 |
| DP1675.10 | 8 | 8 | 0 |
| DP1675.11 | 10 | 10 | 0 |
| DP1675.12 | 10 | 10 | 0 |
| DP1675.13 | 10 | 6 | 4 |
| DP1675.14 | 10 | 10 | 0 |
| DP1675.15 | 8 | 8 | 0 |
| Totally | 139 | 135 | 4 |

TABLE 22
Seed setting of bagged panicles of DP1675 at
T1 generation in Beijing (2^{nd} experiment)

| | Bagged | Seed set | | |
|---|---|---|---|---|
| | panicle | | Semi- | |
| Line ID | number | Fertility | sterility | Sterility |
| DP1675.01 | 9 | 7 | 0 | 2 |
| DP1675.02 | 10 | 10 | 0 | 0 |
| DP1675.03 | 8 | 5 | 2 | 1 |
| DP1675.04 | 10 | 10 | 0 | 0 |
| DP1675.05 | 7 | 6 | 0 | 1 |
| DP1675.06 | 10 | 7 | 0 | 3 |
| DP1675.07 | 9 | 3 | 0 | 6 |
| DP1675.08 | 10 | 3 | 3 | 4 |
| DP1675.09 | 9 | 8 | 0 | 1 |
| DP1675.10 | 8 | 6 | 0 | 2 |
| DP1675.11 | 10 | 9 | 1 | 0 |
| DP1675.12 | 10 | 5 | 0 | 5 |
| DP1675.13 | 8 | 7 | 0 | 1 |
| DP1675.14 | 10 | 7 | 0 | 3 |
| DP1675.15 | 10 | 7 | 1 | 2 |
| Totally | 138 | 100 | 7 | 31 |

TABLE 23
Seed setting of DP1675 at T1 generation
in Beijing (2^{nd} experiment)

| | | Seed set | | |
|---|---|---|---|---|
| | Plant | | Semi- | |
| Line ID | number | Fertility | sterility | Sterility |
| DP1675.01 | 20 | 14 | 1 | 5 |
| DP1675.02 | 19 | 19 | 0 | 0 |
| DP1675.03 | 20 | 14 | 0 | 6 |
| DP1675.04 | 20 | 20 | 0 | 0 |
| DP1675.05 | 20 | 13 | 0 | 7 |
| DP1675.06 | 16 | 11 | 4 | 1 |
| DP1675.07 | 15 | 6 | 1 | 8 |
| DP1675.08 | 20 | 10 | 1 | 9 |
| DP1675.09 | 13 | 10 | 0 | 3 |
| DP1675.10 | 10 | 8 | 2 | 0 |
| DP1675.11 | 13 | 11 | 0 | 2 |
| DP1675.12 | 12 | 6 | 0 | 6 |
| DP1675.13 | 16 | 12 | 0 | 4 |
| DP1675.14 | 13 | 10 | 0 | 3 |
| DP1675.15 | 20 | 15 | 1 | 4 |
| Totally | 247 | 179 | 10 | 58 |

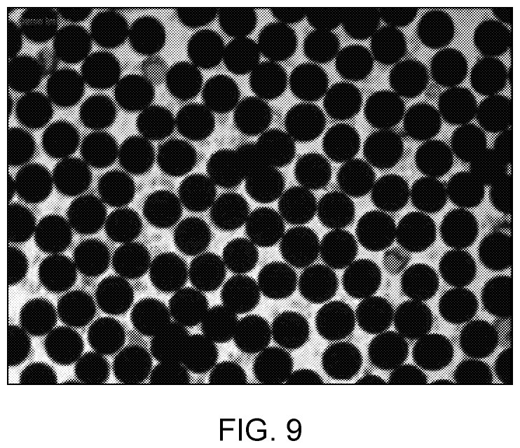

Both pollen and pistil phenotype were observed in the above experiments. The pollen of OsPPT1-1 transgenic rice plants were fertile (FIG. 9), and no abnormal phenotype in pistils was observed with OsPPT1-1 transgenic rice plants. During the field experiments, except for a few individual plants, most of the transgenic rice plants had only a sterile phenotype, and the other phenotypes, include but not limit to, plant height, growth period, yield etc., were normal.

To understand if the phenotypes were impacted by locations and/or environmental factors, the stubbles of OsPPT1-1 transgenic rice were transferred from Beijing to Hainan in October. The pollen of 11 sterile plants at T1 generation were examined by microscope, and the panicles were bagged to measure the seed setting rate. As indicated in table 24, the pollen were fertile and semi-sterile. This might be influenced by the low temperature in Hainan at the stages of booting and/or flowering. Except DP1675.08 line, the seed setting rate of all the other lines were sterile. In a word, the pollen from all plants are fertile or semi-sterile, while the seed setting rate of the bagged plants is sterile or high sterile. The results suggest that OsPPT1-1 is a female-sterile gene.

TABLE 24
Sterile phenotypes of DP1675 at T1 generation
in Hainan (3^{rd} experiment)

| | Seed setting rate | Seed setting rate of |
|---|---|---|
| Line ID | of pollen (%) | bagged plants (%) |
| DP1675.01 | 25 | 0 |
| DP1675.05-1 | 73 | 0 |
| DP1675.05-2 | 83 | 0 |
| DP1675.07-1 | 57 | 0 |
| DP1675.07-2 | 60 | 0 |
| DP1675.08-1 | 83 | 10 |
| DP1675.08-2 | 82 | 5 |
| DP1675.12-1 | 55 | 0 |
| DP1675.12-2 | 77 | 0 |
| DP1675.13 | 50 | 0 |
| DP1675.14 | 90 | 0 |

### Fluorescence Segregation Ratio of OsPPT1-1 Transgenic Rice Seeds

The fluorescence segregation ratio of OsPPT1-1 transgenic rice seeds were measured. The seeds of 165 plants from 15 lines at T1 generation were inspected, using 100 seeds from each plant. The results demonstrated that 12449 of 16460 seeds shown red color under green fluorescence light, while 4011 of 16460 seeds didn't show red color. The ratio of red color seeds to normal seeds was 3.1:1. The results indicated that a single OsPPT1-1 gene was inserted in the OsPPT1-1 transgenic rice genome.

### Cross-Out Results of OsPPT1-1 Transgenic Rice

To further investigate the function of OsPPT1-1 gene, cross-out experiments were performed. The cross-out seeds were validated by PCR with the primers of SEQ ID NO: 24 and SEQ ID NO: 25. The PCR validation results demonstrated that all the cross-out plants contained the OsPPT1-1 gene. The seed setting of the cross-out plants was measured. As shown in table 25, all the plants were fertile.

TABLE 25
Sees sets of cross-out of DP1675 lines at F1 generation

| | | Total | Seed setting | | |
|---|---|---|---|---|---|
| | Gener- | plant | | Semi- | |
| Line ID | ation | number | Fertility | sterility | Sterility |
| DP1675.07♂.1-7-F | F1 | 7 | 7 | 0 | 0 |
| DP1675.07♂.1-9-F | F1 | 8 | 8 | 0 | 0 |
| DP1675.07♂.1-2-F | F1 | 10 | 10 | 0 | 0 |
| DP1675.05♂.1-3-F | F1 | 10 | 10 | 0 | 0 |

The seeds showing red color under green florescence light from the F1 generation were planted in Hainan. As shown in table26, the seed setting rate of the cross-out plants were fertile, semi-sterile and sterile panicles; the ratio of fertile panicles to sterile panicles was about 1.4:1. This might be influenced by the low temperature in Hainan at the stages of booting and/or flowering. These results also demonstrate that OsPPT1-1 is likely a recessive female sterile gene.

TABLE 26
Seed sets of cross-out of DP1675 lines at F2 generation

| | | Total | Seed setting | | |
|---|---|---|---|---|---|
| | Gener- | plant | | Semi- | |
| Line ID | ation | number | Fertility | sterility | Sterility |
| DP1675.07♂.1-7-1 | F2 | 19 | 8 | 5 | 6 |
| DP1675.07♂.1-7-2 | F2 | 20 | 3 | 9 | 8 |
| DP1675.07♂.1-7-3 | F2 | 20 | 9 | 3 | 8 |
| DP1675.07♂.1-9-1 | F2 | 20 | 5 | 9 | 6 |
| DP1675.07♂.1-9-2 | F2 | 20 | 2 | 13 | 5 |
| DP1675.07♂.1-9-3 | F2 | 20 | 0 | 9 | 11 |
| DP1675.07♂.1-2-1 | F2 | 20 | 9 | 9 | 2 |
| DP1675.07♂.1-2-2 | F2 | 20 | 6 | 5 | 9 |
| DP1675.07♂.1-2-3 | F2 | 20 | 12 | 5 | 3 |
| DP1675.05♂.1-3-1 | F2 | 20 | 17 | 3 | 0 |
| DP1675.05♂.1-3-2 | F2 | 20 | 14 | 2 | 4 |
| DP1675.05♂.1-3-3 | F2 | 20 | 10 | 8 | 2 |
| Totally | | 239 | 95 | 80 | 64 |

1. A polynucleotide, comprising: (a) a polynucleotide with nucleotide sequence of at least 85% identity to SEQ ID NO: 1, 4 or 7; (b) a polynucleotide with nucleotide sequence of at least 85% identity to SEQ ID NO: 2, 5 or 8; (c) a polynucleotide encoding a polypeptide with amino acid sequence of at least 90% identity to SEQ ID NO: 3, 6 or 9; or (d) the full complement of the nucleotide sequence of (a), (b) or (c), wherein increasing expression of the polynucleotide in a plant makes plants sterile, and can be used to develop novel seed production technology.

2. The polynucleotide of claim 1, wherein the said polynucleotide comprises the nucleotide sequence of SEQ ID NO: 1, SEQ ID NO: 2, SEQ ID NO: 4, SEQ ID NO: 5, SEQ ID NO: 7 or SEQ ID NO: 8.

3. The polynucleotide of claim 1, wherein the polynucleotide encodes a polypeptide comprising the amino acid sequence of SEQ ID NO: 3, SEQ ID NO: 6 or SEQ ID NO: 9 or an amino acid sequence that is at least 90% identical to SEQ ID NO: 3, SEQ ID NO: 6 or SEQ ID NO: 9.

4. Use of the polynucleotide of claim 1 in a plant to regulate sterility of plants.

5. A recombinant DNA construct comprising the polynucleotide of claim 1 operably linked to at least one heterologous regulatory element.

6. A transgenic plant, plant cell or seed, comprising in its genome a recombinant DNA construct, wherein the recombinant DNA construct comprises the polynucleotide of claim 1 operably linked to at least one regulatory element, wherein the said plant is sterile.

7. The transgenic plant or plant cell of claim 6, wherein the polynucleotide comprises SEQ ID NO: 1, SEQ ID NO: 2, SEQ ID NO: 4, SEQ ID NO: 5, SEQ ID NO: 7 or SEQ ID NO: 8.

8. The transgenic plant or plant cell of claim 6, wherein the polynucleotide encodes a polypeptide comprising the amino acid sequence of SEQ ID NO: 3, SEQ ID NO: 6 or SEQ ID NO: 9.

9. The plant of claim 6, wherein the polynucleotide encoding a Dnak polypeptide or ZOS3-17 polypeptide or PPT1-1 polypeptide is an endogenous polynucleotide.

10. The plant of claim 6, wherein said plant is selected from the group consisting of rice, maize, soybean, sunflower, sorghum, canola, wheat, alfalfa, cotton, barley, millet, sugar cane and switchgrass.

11. A method of making a sterile plant, comprising increasing the expression of the polynucleotide of claim 1.

12. The method of claim 11, wherein the polynucleotide comprises: (a) a polynucleotide with a nucleotide sequence of at least 85% identity to SEQ ID NO: 1, 4 or 7; (b) a polynucleotide with a nucleotide sequence of at least 85% identity to SEQ ID NO: 2, 5 or 8; and (c) a polynucleotide encoding a polypeptide with an amino acid sequence of at least 90% identity to SEQ ID NO: 3, 6 or 9.

13. The method of claim 12, comprising:
(a) introducing into a regenerable plant cell a recombinant DNA construct comprising a polynucleotide operably linked to at least one regulatory element, wherein the polynucleotide encodes a polypeptide having an amino acid sequence of at least 80% sequence identity compared to SEQ ID NO: 3, 6 or 9;
(b) regenerating a transgenic plant from the regenerable plant cell after step (a), wherein the transgenic plant comprises in its genome the recombinant DNA construct; and
(c) obtaining a progeny plant derived from the transgenic plant of step (b), wherein said progeny plant comprises in its genome the recombinant DNA construct and exhibits sterile trait when compared to a control plant not comprising the recombinant DNA construct.

14. The plant of any one in claim 12, wherein the said plant is selected from the group consisting of rice, maize, soybean, sunflower, sorghum, canola, wheat, alfalfa, cotton, barley, millet, sugar cane and switchgrass.

15. A method of identifying one or more alleles associated with sterile trait in a population of rice plants, the method comprising the steps of:
(a) detecting in a population of rice plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO: 3, 6 or 9 or a sequence that is 90% identical to SEQ ID NO: 3, 6 or 9, wherein the one or more polymorphisms in the genomic region encoding the polypeptide or in the regulatory region controlling expression of the polypeptide is associated with sterile trait; and
(b) identifying one or more alleles at the one or more polymorphisms that are associated with sterile trait.

16. The method of claim 15, wherein the one or more alleles associated with sterile trait is used for marker assisted selection of a rice plant with sterile trait.

17. The method of claim 15, wherein the one or more polymorphisms is in the coding region of the polynucleotide.

18. The method of claim 15, wherein the regulatory region is a promoter.

19. A polynucleotide, comprising: (a) a polynucleotide with nucleotide sequence of at least 85% identity to SEQ ID NO: 26, 27, 29, 31, 33, 35, 36, 38, 40, 42, 44, 45, 47, 49 or 51; (b) a polynucleotide encoding a polypeptide with amino acid sequence of at least 90% identity to SEQ ID NO: 28, 30, 32, 34, 37, 39, 43, 46, 48, 50 or 52; or (c) the full complement of the nucleotide sequence of (a) or (b), wherein increasing expression of the polynucleotide in a plant confers female sterility to the plants, and can be used to develop novel seed production technology, wherein said polynucleotide is operably linked to at least one heterologous regulatory element.

20. A method of making a female sterile plant, comprising increasing the expression of a polynucleotide of claim 19.

21. The method of claim 20, wherein the female sterile plant is a rice, maize, or wheat plant.

22. A method of identifying one or more alleles associated with a female sterile trait in a population of maize or wheat plants, the method comprising the steps of:
(a) detecting in a population of maize or wheat plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO: 28, 30, 32, 34, 37, 39, 41, 43, 46, 48, 50 or 52 or a sequence that is 90% identical to SEQ ID NO: 28, 30, 32, 34, 37, 39, 41, 43, 46, 48, 50 or 52, wherein the one or more polymorphisms in the genomic region encoding the polypeptide or in the regulatory region controlling expression of the polypeptide is associated with the female sterile trait; and
(b) identifying one or more alleles at the one or more polymorphisms that are associated with the female sterile trait.
